SEQUENCE LISTING (2 sequences)

SEQ ID NO: 1 - DNA, 68 bp, Articial Sequence
  Synthetic polynucleotide
aaaaaaagatctggctacagcccactagtaaggagaagataaatggagaaaaaaatcactggatatac

SEQ ID NO: 2 - DNA, 34 bp, Artificial Sequence
  Synthetic polynucleotide
aaaaaaggatccaaactcgagttacgccccgccc

(19) United States
(12) Patent Application Publication
(10) Pub. No.: US 2015/0330992 A1
(43) Pub. Date: Nov. 19, 2015

(54) RAPID DISCOVERY AND SCREENING OF ENZYME ACTIVITY USING MASS SPECTROMETRY

(71) Applicants: THE REGENTS OF THE UNIVERSITY OF CALIFORNIA, Oakland, CA (US); SANDIA CORPORATION, Livermore, CA (US)

(72) Inventors: Trent R Northen, Walnut Creek, CA (US); Kai Deng, Albany, CA (US); Tristan P De Rond, Berkeley, CA (US); Pamela P Peralta-Yahya, Emeryvill, CA (US); Xiaoliang Cheng, Albany, CA (US); Jay D Keasling, Berkeley, CA (US)

(73) Assignees: The Regents of the University of California, Oakland, CA (US); Sandia Corporation, Livemore, CA (US)

(21) Appl. No.: 14/651,546

(22) PCT Filed: Dec. 11, 2013
(86) PCT No.: PCT/US2013/074495

§ 371 Date: Jun. 11, 2015

Related U.S. Application Data
(60) Provisional application No. 61/736,409, filed on Dec. 12, 2012.
(60) Provisional application No. 61/738,929, filed on Dec. 18, 2012.
(60) Provisional application No. 61/777,617, filed on Mar. 12, 2013.

(51) Int. Cl. G01N 33/68

(52) U.S. Cl. CPC G01N 33/6848; G01N 2333/90

(57) ABSTRACT

Described herein are methods, systems, and compositions for detecting enzyme activity. In some embodiments, the reaction product(s) are coupled with a mass tag, and the enzyme activity is determiner by analyzing the reaction product(s). The enzyme assays can be performed using mass spectrometry, for example nanostructure-initiator mass spectrometry (NIMS). Also described are methods, systems, and compositions for monitoring enzymatic degradation process of a substrate sample, for example a biomass.

### RELATED APPLICATIONS

The present application claims priority under 35 U.S.C. §119(e) to U.S. Provisional Patent Application No. 61/736,409, filed on Dec. 12, 2012, U.S. Provisional Patent Application No. 61/738,929, filed on Dec. 18, 2012, and U.S. Provisional Patent Application No. 61/777,617, filed on Mar. 12, 2013. The content of each of these related applications is hereby incorporated by reference in its entirety.

### STATEMENT GOVERNMENT RIGHTS

This invention was made with governmental support awarded by the National Institutes of Health under Grant No. 1RC1GM090980-01, and by the U.S. Department of Energy under Contract Nos. DE-AC02-05CH11231, DE-FC02-07ER64494, and DE-AC04-94AL85000. The government has certain rights in the invention. This work is also a collaboration between inventors at Lawrence Berkeley National Laboratory and Sandia National Laboratories for the Joint BioEnergy Institute.

### REFERENCE TO SEQUENCE LISTING

The present application is being filed along with a Sequence Listing in electronic format. The Sequence Listing is provided as a file entitled SEQLISTING.TXT, created Dec. 11, 2013, which is 4 Kb in size. The information in the electronic format of the Sequence Listing is incorporated herein by reference in its entirety.

### BACKGROUND

1. Field

The present application relates generally to the fields of microbiology, molecular biology, biofuel technology, and biomedicine. More specifically, the present application relates to methods, compositions and systems for analyzing and detecting enzymatic activities.

2. Description of the Related Art

Given the biochemical and biotechnological significance of enzymes, high-throughput assays for the detection of enzymatic activity are in high demand. High-throughput enzyme activity assays are critical in the drug and biomarker discovery processes, and can be a valuable tool for functional gene annotation. They figure prominently in directed evolution experiments, where libraries of enzyme variants are screened for improved substrate specificity, thermal stability or other beneficial properties.

High-throughput enzyme activity assays can help alleviate experimental bottlenecks, but few generally-applicable technologies are currently available. Some enzymatic reactions can be coupled to a spectroscopic readout by employing chromogenic substrates or by indirectly measuring product formation via a coupled assay or biosensor. Such assays are fast, but applicable to only the narrow range of biochemical transformations for which methods have been developed. On the other hand, liquid chromatographic-mass spectrometry-based assays are more universal, but require lengthy chromatographic separations to avoid spectral complexity and ionization suppression, drastically reducing experimental throughput. In one technology, known as Nimzyme, a specialized enzyme substrate probe is presented to a biological mixture potentially exhibiting enzymatic activity, followed by an in situ enrichment step using fluorous interactions and nanostructure-initiator mass spectrometry. Nimzyme technology allows high-throughput assay for enzyme activity, but requires chemical synthesis of custom substrate analogs as substrate probes and is limited by the solubility of the substrate. There is a need for simplifying the production of enzyme substrate probes and methods that do not rely on specialized enzyme substrate probes and are applicable to substrates are not soluble or has low solubility, such as solid biomass.

### SUMMARY

The present application relates to methods, compositions, and systems for detecting enzymatic activities.

Some embodiments provide a method for detecting the activity of an enzyme sample, wherein the method includes: (a) incubating a substrate sample with an enzyme sample to form a reaction mixture, wherein a substrate sample containing one or more substrates for an enzyme sample, and wherein the reaction mixture contains one or more reaction products; (b) coupling the one or more reaction products with a mass probe to form tagged reaction products: and (c) detecting the enzymatic activity of the enzyme sample by analyzing at least one of the one or more reaction products. In some embodiments, the substrate sample is a biological sample, an environmental sample, or a combination thereof. In some embodiments, the substrate sample comprises a biomass, a crude lysate, or a cell culture.

Some embodiments provide a method for monitoring enzymatic degradation process of a substrate sample, wherein the method includes (a) incubating a substrate sample with an enzyme sample to form a reaction mixture, wherein the reaction mixture contains one or more reaction products; (b) coupling the one or inure reaction products with a mass probe to form tagged reaction products; and (c) determining the extent of enzymatic degradation of the substrate sample by analyzing at least one of the one or more reaction products. In some embodiments, the method comprises repeating steps (b) and (c) one or more times to determine the process of enzymatic degradation of the substrate sample. In some embodiments, the method further includes adjusting the composition of the enzyme sample before repeating steps (b) and (c). In some embodiments, determining the extent of enzymatic degradation of the substrate sample comprises analyzing the at least one of the one or more reaction products.

Some embodiments provide a method for detecting the activities of a plurality of enzymes in a multiplexed assay, wherein the method includes (a) providing a substrate sample containing substrates for a plurality of enzyme; (b) incubating the substrate sample with the plurality of enzyme to form a reaction mixture, wherein the reaction mixture obtains reaction products; (c) coupling the reaction products with a mass probe to form tagged reaction products; and (d) detecting activities of the plurality of enzymes by analyzing the reaction products. In some embodiments, the substrate sample is a biological sample, an environmental sample, or a combination thereof.

### BRIEF DESCRIPTION OF THE DRAWINGS

FIG. 1A is a schematic illustration of a click chemistry reaction example where an alkyne-containing molecule A reacted with an azide-containing molecule B form a conjugate of A-B crosslinked with a triazole moiety (using a Copper (Cu) catalyst). FIG. 1B is a schematic illustration of a non-limiting example of the Whole cell “Probing Enzymes with ‘Click’-Augmented NIMS” (PECAN) method.

FIG. 2 is a schematic illustration of a non-limiting synthesis scheme for attaching an azide to the fluorous tag for use with the PECAN method.

FIG. 3 is a schematic illustration of a fluorous tag-based detection method as applied to a glycohydrolase reaction.

FIG. 4A is a schematic illustration of a high throughput surface-based enzyme assay. FIG. 4B shows the reaction of lactose substrate S incubated with Beta-1,4-galactosidase to form reaction product P.

FIGS. 5A-C show exemplary mass spectra (from mass spectrometry (MS) analysis) of the reaction products resulted from the reaction between glycoside hydrolases and various types of pre-treated switchgrass.

FIG. 6 shows time-resolved catalyses by mass spectrometry (MS) with a diagnostic probe (NIMS-Cellotetraose). Different enzymes show different reaction time-courses.

FIG. 7 shows non-limiting examples of oxime products of glucose and cellobiose.

FIG. 8 are calibration curves for reaction of oligosaccharides using the reaction condition A listed in Table 1.

FIGS. 9A-D are schematic illustration of oxime-NIMS analysis of glycosyl hydrolase enzyme activities. FIG. 9A shows that glycosyl hydrolases are a diverse family of enzymes that play critical roles in plant cell wall remodeling, function of the gut microbiome and production renewable fuels. FIG. 9B shows that glycosyl hydrolases act on cellulose and hemicellulose to release complex mixtures of mono- and oligosaccharides. FIG. 9C shows oxime-based derivatization of hydrolysis products results in favorable analytes for direct nanostructure-initiator mass spectrometry. FIG. 9D illustrates that oxime NIMS analysis can allow rapid functional and kinetic analysis of in vitro translated enzymes.

FIG. 10 is a plot showing the results of steady-state kinetic analysis of the reaction of Alicycyclobacillus acidocaldarius β-glucosidase with varied cellobiose concentrations.

FIGS. 11A-B are plots showing time courses for reaction of monofunctional CelB (A) and multifunctional CelEcc_CBM3a (B) with IL-SG. Cellobiose (g2, green dotted line) representing the major product. Total soluble hexose sugars (open circles, solid green line) and remaining cellulose (86%, open squares, solid black line, calculated by subtraction of detected total hexoses from total glucan added to the reaction). Cellulose (open squares, dotted green line) and hemicellose fractions (open up triangles, dotted orange line). Total soluble hexose sugars (open circles, solid green line) and total soluble pentose sugars (open diamonds, solid orange line).

FIGS. 12A-B are plots showing kinetic schemes for the enzymatic hydrolysis of cellulose and hemicellulose. FIG. 12A shows cellulose hydrolysis leading to the release of soluble hexose sugars and subsequent hydrolysis reactions. FIG. 12B shows hemicellulose hydrolysis leading to soluble pentose sugars and subsequent conversions of the soluble oligosaccharides.

FIG. 13 is a plot showing time course for the formation of individual reaction products during the reaction of CelB with IL-SG. A, time course for formation of hexose products: cellulose fraction in unreacted biomass (green solid circles); sum of products (cyan squares); cellobiose (g2, purple down triangles); glucose (g1, blue diamonds); cellotriose (g3, black up triangles). Solid lines were generated by solving differential equations corresponding to the kinetic scheme of FIG. 12A. Apparent rate constants for individual kinetic steps are shown in Table 4.

FIG. 14A-F are plots showing time course for formation of individual reaction products during the reaction of multifunctional CelEcc_CBM3a with IL-SG (FIGS. 14A-C) and binary enzyme mixtures of CelEcc-CBM3a and XynY with IL-SG (FIG. 14D-F). FIG. 14A: time course for formation of hexose products: cellulose fraction in unreacted biomass (green solid circles); sum of products (cyan squares); cellobiose (g2, purple down triangles); glucose (g1, blue diamonds); cellotriose (g3, black up triangles). FIG. 14B: time course for formation of pentose products: hemicellulose fraction in unreacted biomass (green solid circles); pentotriose (p3, black up triangles); pentobiose (p2, purple down triangles); pentose (p1, blue diamonds). FIG. 14C: sum of products (cyan squares); pentotetraose (p4, red squares); pentopentaose (p5, brown open circles). Solid lines in B and C were obtained by solving differential equations corresponding to the kinetic scheme in FIG. 12B. FIG. 14D: time course for formation of hexose products: cellulose fraction in unreacted biomass (green solid circles); sum of products (cyan squares); cellobiose (g2, purple down triangles); glucose (g1, blue diamonds); cellotriose (g3, black up triangles). FIG. 14E: time course for formation of pentose products: hemicellulose fraction in unreacted biomass (green solid circles); sum of products (cyan squares); pentobiose (p2, purple down triangles); pentose (p1, blue diamonds); pentotriose (p3, black up triangles). FIG. 14F: time course for formation of pentotetraose (p4, red squares); pentopentaose (p5, brown open circles).

FIG. 15A-B are plots showing time course for formation of individual products during the reaction of XynY with IL-SG. FIG. 15A shows time course for formation of pentose products: hemicellulose fraction in unreacted biomass (green solid circles); pentobiose (p2, purple down triangles); pentose (p1, blue diamonds); pentotriose (p3, black up triangles). FIG. 15B: sum of products (cyan squares); pentotetraose (p4, red squares); pentopentaose (p5, brown open circles). Solid lines in A and B were obtained by solving differential equations corresponding to the kinetic scheme in FIG. 12B. Apparent rate constants for individual kinetic steps are shown in Table 4.

FIG. 16A is a schematic illustration of traditional enzyme assay. FIGS. 16B-D show a schematic illustration of a non-limiting exemplary application of the Nimzyme technology.

FIG. 17A shows synthesis of Nimzyme substrates using to 1 and 2 as alkylating agents, and NIMS spectra of the products purified only by F-SPE (for 1), or liquid-liquid extraction (for 2). Yields reported are combined yields for alkylation, purification, and (for 1) deprotection. Key: THF=tetrahydrofuran; DMF=N,N′-dimethylformamide. FIGS. 17B-E show detection of chloramphenicol acetyltransferase (CAT) activity in cell lysate using Nimzyme. FIG. 17B shows that CAT catalyzes the O³-acetylation of chloramphenicol. FIG. 17C shows that chloramphenicol analog 11 was synthesized through the alkylation of (1R,2R)-2-Amino-1-(4-nitrophenyl)-1,3-propanediol with 1 according to the methodology described herein. The structure of the moiety abbreviated Nz is shown in FIG. 17A, FIG. 17D shows exposure of 11 to control lysate, followed by the Nimzyme workflow shown in FIG. 16 shows a clean mass peak corresponding to 11 (expected M+H, m/z=896.26). FIG. 17E shows exposure of 11 to lysate of E. coli having expressed CAT, followed by Nimzyme, shows a mass shift of exactly one acetyl unit relative to 11 (expected M+H, m/z=938.26). Key: THF=tetrahydrofuran; TFA=trifluoroacetic acid; DMF=N/N′-dimethylformamide.

FIG. 18A-I show the spectral peak for each of the compounds 3-11: FIG. 18A is the spectral peak for compound 3—O-Nz-borneol; FIG. 18B is the spectral peak for compound 4—O-Nz-cholesterol; FIG. 18C is the spectral peak for compound 5—N-Nz-tryptamine; FIG. 18D is the spectral peak for compound 6—N-Nz-propanolol; FIG. 18E is the spectral peak for compound 7—O-Nz-vanillin ether; FIG. 18F is the spectral peak for compound 8—6-Nz-flavone ether; FIG. 18G is the spectral peak for compound 9—Ibuprofen Nz ester; FIG. 18H is the spectral peak for compound 10—Nalidixic acid Nz ester; FIG. 18I is the spectral peak for compound 11—(1R,2R)-N-Nz-1-(4-nitrophenyl)propane-1,3-diol.

FIG. 19 shows a probe with various moieties enclosed in labeled boxes which correspond to carbons and protons attached to carbon and which show up in the spectra of the tagged compounds. The spectra of the tagged compounds 14, 15, 16, 1 and 2 are shown in FIGS. 20-21.

FIG. 20 shows ¹H NMR of tagged compounds 14, 15, 16, 1 and 2 stacked from bottom to top. Colored boxes indicate peaks due to proton corresponding to the chemical moieties enclosed in the same labeled box in FIG. 19. Key: *=artifact/solvent impurity; NH=presumed amide proton; F=proton due to Fmoc group.

FIG. 21 shows ¹³C NMR of tagged compounds 14, 15, 16, 1 and 2 stacked from bottom to top. Colored boxes indicate peaks due to carbons corresponding to the chemical moieties enclosed in the same labeled box in FIG. 19. Key: *=artifact solvent impurity; F=proton due to Fmoc group.

FIG. 22A shows the reaction of pyruvate with the probe and the resulting tagged pyruvate. FIG. 22B shows a metabolic reaction of an N-linked glycoprotein the tagged pyruvate to produce a tagged N-linked glycoprotein.

FIG. 23 shows a non-limiting embodiment of the method described herein. The substrates are derivatized and the biomass tag and an ionizable moiety are attached to the substrate through a water soluble linker. The tagged substrate are then reacted with the enzyme in crude lysate and then spotted onto a NIMS surface and analyzed.

FIG. 24 is a schematic illustration for a traditional enzyme assay and the use of whole cell “Probing Enzymes with ‘Click’-Augmented NIMS” (PECAN) method for detecting enzyme activity in cells or cell lysate.

FIG. 25 shows NIMS spectra for the detection of chloramphenicol acetyltransferase (CAT) activity in intact E. coli cell culture using whole cell PECAN method.

FIG. 26 shows NIMS spectra for the detection of cytochrome P450 activity in E. coli cell culture using whole cell PECAN method.

FIG. 27 shows a non-limiting example of the chemical condensation reaction between a mass probe and ketone or aldehydes.

FIGS. 28A-D are NIMS spectra for four ketone/aldehyde compounds coupled with mass probe(s): (A) 2-acetoxy-3-butanone, (B) n-butyl aldehyde, (C) Ethyl isobutyl ketone, and (D) 4-methyl-pentanone.

### DETAILED DESCRIPTION

The description that follows illustrates various embodiments of the subject matter disclosed herein. Those of skill in the art will recognize that there are numerous variations and modifications of the subject matter provided herein that are encompassed by its scope. Accordingly, the description of certain embodiments should not be deemed to limit the scope of the present application.

In addition, in the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are contemplated and make part of this disclosure.

The present application relates to methods, systems and compositions for detecting enzyme activity in an enzyme sample. Activities of one or more of the enzymes in the enzyme sample can be detected by incubating a substrate sample to be tested for enzymatic activity with the enzyme sample to obtain reaction products, coupling the reaction products with a mass probe to form tagged reaction products, and analyzing the reaction products. The presence and/or amount of reaction products can be analyzed, for example by mass spectrometry (MS) (e.g., nanostructure-initiator mass spectrometry (NIMS)) to determine the activity of one or more enzymes of the enzyme sample, and thus determine the presence of the one or more enzymes in the sample.

The methods, compositions and systems described herein can be used in diverse fields such as microbiology, development of biofuels, conversion of biomass, and various biological and biomedical applications. For example, methods, systems, and compositions described herein can provide simultaneous identification and characterization of the enzymatic activities of one or more enzymes directly from crude biological or environmental samples. The methods, systems, and compositions described herein can also be used for biological applications, such as monitoring enzymatic degradation process of a substrate sample, for example a biomass, and high-throughput screening of enzyme library. Because enzymatic hydrolysis of polysaccharides into fermentable sugars by glycoside hydrolases is an important step in the conversion of biomass to lignocellulosic biofuels, the methods, systems and compositions described herein are useful for development of biofuels. The methods, systems, and compositions described herein can also be used to detect enzyme activity in cell lysates and intact cells.

The methods, compositions and systems disclosed herein can be used for high-throughput enzyme activity determination based on, for example mass spectrometry techniques.

### Enzyme Samples

The methods, systems, and compositions described herein can be used to determine the activity of an enzyme sample. As used herein, an “enzyme sample” refers to a sample containing one or more enzymes. In some embodiments, the enzyme sample contains only one enzyme. In some embodiments, the enzyme sample contains two more enzymes. When the enzyme sample contains two or more enzymes, the activity of the enzyme sample can be the activity of one of the enzymes contained in the enzyme sample, or the activities of some of the enzymes contained in the enzyme sample, or the activities of all of the enzymes contained in the enzyme sample. In some embodiments, an enzyme sample is a mixture of two or more enzymes, for example an enzyme cocktail in which each of the enzyme contained is present in a predetermined amount. In some embodiments, the enzyme has an activity to change the mass of a substrate.

Various enzymes can be analyzed by the methods, systems and compositions described herein. For example, the enzyme can be a monofunctional enzyme or a multifunctional enzyme. As used herein, a “multifunctional enzyme” refers to an enzyme that has at least two distinct enzymatic activities. In some embodiments, the two distinct enzymatic activities are associated with two distinct active sites on the enzyme. The enzyme can be a naturally-occurring enzyme, or a modified enzyme. For example, the enzyme can be a modified or non-naturally occurring enzyme which has been mutated or genetically engineered to provide different, altered (e.g., decreased or improved) function and/or enzymatic activity. In some embodiments, the multifunctional enzyme is an enzyme capable of simultaneous releasing of hexose and pentose products.

The types of enzymes contained in the enzyme sample are not particularly limited. For example, the enzyme can be a carbohydrate-active enzyme, or a non-carbohydrate-active enzyme. As used herein, a “non-carbohydrate-active enzyme” refers to an enzyme that is not involved in the synthesis, metabolism, and transport of carbohydrates. Non-limiting examples of enzyme that can be analyzed by the methods, systems and compositions described herein include transferases, hydrolases, lyases, ligases, isomerases, and oxidoreductases. For example, the transferases can be glycosyltransferases, methyltransferases, acyltransferases, sulfurtransferases, transketolases, riboflavin synthase, polymerase, transaminase, selenotransferase, molybdenunitransferase, and sulfotransferases. Examples of hydrolase include, but are not limited to, lipases, phosphatases, glycoside hydrolases, and proteases. A non-limiting example of oxidoreductases is polyketide synthase.

The enzyme can be a carbohydrate-active enzyme, for example, the enzyme can be involved in sugar modification. The enzyme can be, for example, a glycoside hydrolase, polysaccharide lysase, or glycosyltransferases. Non-limiting examples of glycoside hydrolase include transglycosidases, alpha-glucan lyase, NAD-dependent glycoside hydrolases, and phosphorylase. In some embodiments, the enzyme is involved in sugar modification. For example, the enzyme can have an activity related to changing the chain length of a sugar head group. In some embodiments, the enzyme can reduce the chain length of a sugar substrate. For example, enzymes include enzymes that cleave off one more sugar monomers (glycohydrolases) or enzymes that extend the sugar head group by attaching one or more sugar units (glycotransferases). Examples of enzyme include, but are not limited to, glycohydrolases, glucosidase, cellulose, glycotransferases, endoglucanases, exoglucanases, and hemicelluases. In some embodiments, the enzyme can degrade plant cell wall and/or lignin. For example, the enzyme can also be amylases, xylinases, fumarase, or lactases. In some embodiments, the sugar includes cellulose, hemicellulose, xylose, cellobiose, cellotetraose, xylobiose, or any combination thereof. In some embodiments, the enzyme is a laccase or peroxidase. In some embodiments, the enzyme is a glycoside hydrolase, a polysaccharide lyase, or a glycosyltransferase. In some embodiment, the enzyme can produce pentose products. In some embodiment, the enzyme can produce hexose products.

In some embodiments, the enzyme is involved in degrading sugar. Some embodiments are related to detecting activity of an enzyme involved in degrading plant cell wall material. For example, glycoside hydrolases are important for the development of biofuels from lignocellulosic biomass: long-chain polysaccharides from plant cell walls are enzymatically hydrolyzed and the resulting sugar monomers are fermented into ethanol or advanced biofuels. Three major components forming plant cell walls that are deconstructed include the polysaccharides cellulose and hemicellulose, and the highly phenolic macromolecule lignin. Cellulose is comprised of linear chains of β-1,4-linked D-glucose units, while hemicellulose consists mainly of mixtures of pentoses with D-xylose, and D-arabinose being the most abundant. Cellulose is hydrolyzed into glucose through the concerted action of at least three known classes of enzymes collectively referred to as cellulose: endoglucanases, exoglucanases, and β-glucosidases. Without being bound by any particular theory, endoglucanases randomly produce free ends from cellulose fibrils that are further degraded by exoglucanases that release cellobiose, which in turn is hydrolyzed by β-glucosidases into glucose. Hemicelluloses are degraded by a complex class of multi-domain enzymes known as hemicellulases. Lignin gets broken down by “ligninases”, e.g. laccases or lignin peroxidases. Some embodiments disclosed herein relate to detection of these enzymes involved in degrading or breaking down plant cell wall.

The enzyme can, in some embodiments, include cellulase, which includes but is not limited to endoglucanases (endocellulases) for example, endo-1,4-beta-glucanase, carboxymethyl cellulose (CMCase), endo-1,4-beta-D-glucanase, beta-1,4-glucanase, beta-1,4-endoglucan hydrolase, and celludextrinase; exoglucanases (exocellulases); and beta-glucosidases. In some embodiments, the enzymes are cellulose enzymes identified by the Enzyme Commission number EC 3.2.1.4, which is herein fully incorporated by reference in its entirety for examples of enzymes contemplated herein.

The enzyme can, in some embodiments, include ligninase, which includes but is not limited to lignin peroxidase, manganese peroxidase, laccase, and cellobiose dehydrogenase. In some embodiments, the enzymes are ligninase enzymes identified by the Enzyme Commission number EC 1.14.99, which is herein fully incorporated by reference in its entirety for examples of enzymes contemplated herein.

The enzyme can be, in some embodiment, a non-carbohydrate-active enzyme. For example, the enzyme can be an enzyme that is not involved in degrading sugars. Non-limiting of these enzymes include acetyltransferases, transferases, carboxylases, isomerases, anhydrases, dismutases, catalases, esterases, lactamases, phosphatases, kinases, reductases, oxidases, proteases, hydroxylases, polymerases, dehydrogenases, trypsin, lipases, synthetases, ligases and restriction enzymes. For example, the enzyme can be a lipase, a protease, or a phosphatase.

In some embodiments, the enzyme is involved in catalyzing the formation or hydrolysis of lipids. Examples of lipase include, but are not limited to, Lipase AP4, Lipase AP6, Lipase F-AP15, Lipase OF, Lipase AP12, Lipase M-AP5, Lipase M-AP10, Lipase M-AP20, Lipase Saiken, Lipase PS, Lipase MY, and Lipase B.

In some embodiments, the enzyme is involved in conducting proteolysis. For example, the enzyme can be a protease, such as serine protease, a threonine protease, cysteine protease, an aspartate protease, a metalloprotease, and a glutamic acid protease.

In some embodiments, the enzyme is involved in dephosphorylation. For example, the enzyme can be a phosphatase, for example, a cysteine-dependent phosphatase, or a metallo-phosphatase. In some embodiments, the enzyme is a protein serine/threonine phosphatase. Non-limiting examples of phosphatase include tyrosine-specific phosphatases, serine-/threonine-specific phosphatases, dual specificity phosphatases, histidine phosphatase, and lipid phosphatase.

In some embodiments, the enzyme is involved in catalyze phosphorylation reactions. For example, the enzyme can be a phosphortransferase. For example, the enzyme can be a phosphotransferase with an alcohol group as acceptor (identified by the Enzyme Commission number EC 2.7.1), a phosphotransferases with a carboxy group as acceptor (EC 2.7.2), a phosphotransferases with a nitrogenous group as acceptor (EC 2.7.3), a phosphotransferases with a phosphate group as acceptor (EC 2.7.4), a diphosphotransferase (EC 2.7.6), or a phosphotransferases with paired acceptors (EC 2.7.9). In some embodiments, the enzyme is a kinase identified by the Enzyme Commission number EC 2.7, for example, a protein kinase, a lipid kinase, or a nucleoside-phosphate kinase.

In some embodiments, the enzyme is an oxidoreductases, for example a monooxygenase (e.g., cytochrome P450 monooxygenases). In some embodiments, the enzyme is a acetyltransferase, for example a chloramphenicol acetyltransferase (CAT). In some embodiments, the non-carbohydrate-active enzyme is a lipase, polyketide synthase, methyltransferase, acetyltransferase, or protease.

The enzyme sample can optionally include one or more additional components, for example, co-enzymes, cofactors, inhibitors or catalysts of one or more enzymes. In some embodiments, the enzyme sample does not contain co-enzyme, cofactor, enzyme inhibitor, or catalyst.

The enzyme sample can be an aqueous solution containing one or more enzymes. The enzymes can be purified enzymes from organisms (e.g., plants, bacteria, or animal), or raw or purified enzymes produced by recombinant techniques. For example, the enzyme sample can be a product from cell-free translation (for example, the cell-free translation method described in Takasuka et al. Method in Molecular Biology, The Humana Press Inc., Totowa, N.J., 2013). In some embodiments, the enzyme sample is a cell culture, a tissue culture, tissue extract, or a cell lysate. The types of the cell lysate or tissue extract can vary, for example, the cell lysate can be crude, desalted, or clarified. In some embodiments, the enzyme sample can comprise whole cells. As would be appreciated by one of ordinary skill in the art, some types of substrates (e,g., hydrophobic substrates) can pass through the cell watt or membrane of the whole cell (e.g., a bacterial cell membrane or wall) to get into the inside of the cell. After the substrates permeate into the whole cell, the substrates can contact and interact with one or more enzymes inside the whole cell. In some embodiments, the substrate interacts with the enzyme(s) inside the whole cells to form reaction products.

### Substrates Samples

As used herein, a “substrate” refers to a molecule that can be converted into reaction product(s) by enzymes during an enzymatic reaction, and enzymes are generally selective and specific for their substrates. In some embodiments, the substrate is a molecule that can be acted upon by an enzyme. For example, in some embodiment, a chloramphenicol analog was used as a substrate to detect chloramphenicol acetyltransferase (CAT) activity in crude cell lysate.

The types of the substrate sample that the methods, systems and compositions described herein can be used to detect activity of enzymes are not particularly limited. The substrate sample can be a crude sample or a purified sample. The types of the substrate sample can vary, for example the sample can be a biological sample or a clinical sample. Examples of biological or clinical sample include, but are not limited to, cells, cellular lysates, cellular extracts, tissue extracts, and bodily fluids. The substrate sample can also be an environmental sample. For example, microbial communities (e.g., fungi or bacteria) capable of growing on lignocellulose have gained increasing attention as sources for discovering glycoside hydrolases. As such, some embodiments described herein relate to detecting enzyme activities in enzyme samples suspected of containing such microbial communities. Non-limiting examples of substrate samples that can be assayed in some embodiments described herein include an organic matter (e.g., a plant matter), wood, crops (e.g., food crops), leaves, paper waste, soil, compost, a native glycan, agriculture waste (e.g. livestock waste), mulch, dirt, clay, garbage, or any combination thereof. In some embodiments, the substrate sample comprises one or more purified enzyme substrates.

The solubility of the substrate sample can vary. The substrate sample, in some embodiments, is soluble in water or aqueous buffers. In some embodiments, the substrate sample comprises enzyme substrate soluble in water or aqueous buffers. In some embodiments, the substrate sample is a solid matter that is substantially insoluble in water or aqueous buffer. In some embodiments, one or more of the substrate in the substrate sample is linked to tags that interact with a surface of a NIMS chip. In some embodiments, the substrate includes a sugar head group linked to a hydrophobic tag, which is capable of interaction with a hydrophobic NIMS chip surface. For example, the substrate can include a sugar head group linked to a perfluorinated tag that interacts with a NIMS chip surface having a perfluorinated coat. Head groups can be sugar monomers, oligomers, or (branched) multimers. In other words, head groups can include monosaccharides, disaccharides, polysaccharides, and oligosaccharides. Examples of monosaccharides, disaccharides, polysaccharides, and oligosaccharides that can be used as head groups in substrate analogs are known in the art. In some embodiment, the substrate sample comprises one or more substrates that are amphiphilic and thus soluble in water or aqueous buffers. Without being bound by any particular theory, dissolving the tagged substrate is possible because they form supramolecular amphiphilic assemblies (e.g., micelles, liposomes, vesicles, colloids, etc.), where the hydrophobic fluorous tail is “shielded” from the aqueous solution, whereas sugar head groups remain accessible for the enzymes' active sites.

In some embodiments, the substrate sample is cultivated and extracted for analysis of enzymatic activity using standard techniques available in the field. For example, environmental samples can be inoculated and grown in liquid cultures containing a biomass feedstock such as switchgrass. The supernatants of the liquid cultures can be collected for analysis and detection of enzyme activity as described herein.

In some embodiments, the sample can be a biomass, for example a plant biomass. The term “biomass”, as used herein, refers to any biological material derived from living, or recently living organisms. For example, the biomass can be plants or plant-derived materials called lignocellulosic biomass. The biomass can comprise native oligosaccharides, heterogeneous biomass substrates, or a combination thereof. In some embodiments, the biomass is substantially or partially deconstructed. For example, the substrate sample can be a heterogeneous mixture of reaction products from biomass deconstruction because of enzymatic, mechanical or chemical treatment. The biomass may contain the entire plant or a portion of the plant such as the fruits, the stems, leaves, roots, shells, seeds, or any combination thereof. The biomass can be a biofuel crop. Non-limiting examples of biofuel crop include switchgrass, maize, cashew nut, oats, lupin, kenaf, calendula, cotton, hemp, soybean, coffee, flax, hazelnuts, euphorbia pumpkin, coriander, mustard, sesame, camelina, safflower, rice, tung tree, sunflowers, cocoa, peanut, rapeseed, olives, castor beans, pecan nuts, jojoba, jatropha, macadamia nuts, brazil nuts, avocado, coconut, Chinese tallow, oil palm, and algae. In some embodiments, the substrate sample is switchgrass. For example, the substrate sample can he ethanol-extracted switchgrass (SC), ammonia fiber expansion treated switchgrass (AFEX-SC), 1-ethyl-3-methylimidazolium acetate ([C₂mim][OAc]) treated switchgrass (IL-SG), or a combination thereof.

The biomass may be used in any suitable form in the methods described herein. For example, the biomass can be collected from the fields and directly used in their natural form. The biomass can also be pretreated before being used in the method described herein. The methods and conditions under which the biomass is pretreated are not particularly limited. The biomass can be pretreated mechanically, biologically, chemically, or in any combination thereof. For example, the biomass can be pretreated by drying under the sun or by a machine, cutting into smaller pieces, smashing, or grinding. As another example, the biomass can be treated by one or more chemical agents, including but not limited to ethanol, alkaline and ionic liquids.

In the methods described herein, the enzymatic reaction, in some embodiments, can be carried out in microliter scale at enzyme to substrate ratio. For example, the reaction can be carried out at 8-20 mg/enzyme/g substrate (e.g., biomass).

Any substrates that can be modified by one or more of the enzymes described herein can be used in the methods, systems, and compositions disclosed herein as an enzyme substrate for the detection of enzymatic activity. In some embodiments, at least one of the substrate in the substrate sample is carbohydrate. In some embodiments, the substrate sample does not contain any carbohydrate substrate.

Examples of the substrate include, but are not limited to, carbohydrates; organic acids, such as pyruvate; compounds containing one or more ketone functional groups, such as ketone; compounds containing one or more aldehyde functional groups, such as aldehyde; polyketides; polypeptides; lipids; nucleic acids; glycopeptide N-linked glycopeptide), or derivatives thereof. Non-limiting examples of carbohydrate include hexoses, such as cellobiose and cellotetraose, or pentoses, such as xylobiose. Other sugars include, without limitation, glucose, fructose, galactose, mannose, maltose, sucrose, lactose, arabinose, xylose, and rhamnose. The enzyme substrate can also be, for example, chloramphenicol or an analog thereof, and isobutyryl-CoA or an analog thereof. The enzyme substrate can also be a substrate for acetyltransferase (e.g., CAT), a substrate for cytochrome P450, or an analog thereof. In some embodiments, the substrate comprises alkyne. In some embodiments, the substrate comprises azide.

In some embodiments, the enzyme substrate is a lipid, for example, a fatty lipid, a glycerolipid, a glycerophospholipid, a prenol lipid, a saccharolipid, a polyketide, or a combination thereof. In some embodiments, the enzyme substrate can be phenolic substrates degradable by ligninases. In some embodiments, the substrate is a polysaccharide. In some embodiments, the substrate is an oligosaccharide with one, two, three, four, five, six, even, or more sugar monomers. In some embodiments, the substrate is a ketone or aldehyde. In some embodiments, the substrate contains at least one ketone group, one aldhyde group, or both.

In some embodiments, enzyme substrates are linked to tags that can interact with a mass spectrometry (MS) surface, for example surface of a NIMS chip. For example, the substrate can be linked to a perfluorinated tag that is capable of interacting with a MS surface (e.g., NIMS chip surface) having a perfluorinated coat.

### Coupling of Reaction Products and Mass Probes

After completing an enzymatic reaction under the desired conditions, the, reaction products can be coupled with a mass probe to form tagged reaction products. The reaction, in some embodiments, can be quenched, e.g., with methanol which denatures all enzymes. For analysis of the occurring enzymatic reactions, small samples volumes (e.g., one microliter and below) can be applied to (e.g., spotted onto) a mass spectrometry surface (e.g., a NIMS chip surface). In the case of a NIMS chip, the nanostructured chip can be coated with ultrathin liquid layers of perfluorinated (di)siloxanes. The fluorous tails of the tag can interact with the chip surface via fluorous-phase-interactions, so that the tagged reaction product can stay on the chip surface. In a “chromatographic” step, all other components of the reaction sample that do not interact with the chip surface can be washed away, or simply be pipetted off, while the reaction product stick to the surface. Analysis of enzymatic activities in the mass spectrometer can be performed based on the presence and amount of the reaction product ions. In some embodiments, an internal standard (e.g., an isotope labeled compound or an analog for the reaction product) is used for determining the presence and amount of the reaction product.

In some embodiments, the reaction product is an oligosaccharide or a monosaccharide. The oligosaccharide can have, for example, two, three, four, five, six, seven, eight, nine, ten, or more sugar monomers. For example, the reaction product can be a hexose (including, but not limited to glucose, cellobiose, cellotriose, and cellotetraose), or pentoses (including, but not limited to xylose, xylobiose, xylotriose, and xylotetraose). The reaction product can also be, for example, glucose, fructose, galactose, mannose, maltose, sucrose, lactose, arabinose, xylose, and rhamnose. In some embodiments, the reaction product is a polyketide. In some embodiments, the reaction product is a compound one or more ketone and/or aldehyde function group. For example, the reaction product can be a ketone or an aldehyde. The reaction product can comprise alkyne azide.

In some embodiments, the reaction products can be coupled with a mass probe. In some embodiments, the mass probe is hydrophobic. For example, the mass probe can be a fluorous probe. In some embodiments, the mass probe comprises a perfluoronated affinity moiety. In some embodiments, the hydrophobic probe is a perfluorinated probe. The fluorous probe can be formed by several perfluorinated chemical structures, e.g., aliphatic carbon chains, phenyl rings, etc. For example, fluorinated aliphatic molecules, such as (heptadecafluoro-1,1,2,2-tetrahydrodecyl)-dimethylchlorosilane (“F17”) and bis(tridecafluoro-1,1,2,2-tetrahydrooctyldimethylsiloxy)-methylchlorosilane (“F26”), can be used in synthesis of a fluorous probe. In some embodiments, the mass probe comprises a moiety of heptadecafluoro-1,1,2,2-tetrahydrodecyl (F17). In some embodiments, the mass probe comprises a hydroxylamine, alkyne, or an azide. Examples of the mass probe include, but are not limited

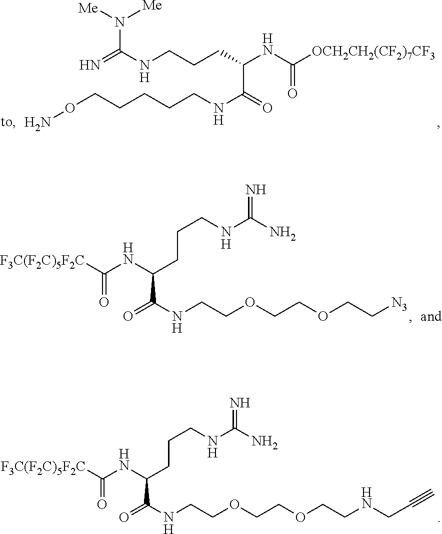

A perfluorinated probe can interact with a hydrophobic MS surface (e.g., NIMS chip surface). In some embodiments, the mass spectrometry surface has a perfluorinated coat. Various initiators including but not limited to lauric acid, polysiloxanes, chlorosilanes, methoxy and ethyoxy silanes, fluorous siloxanes, and silanes can be used for mass spectrometry surface (e.g., NIMS chip surface) coating.

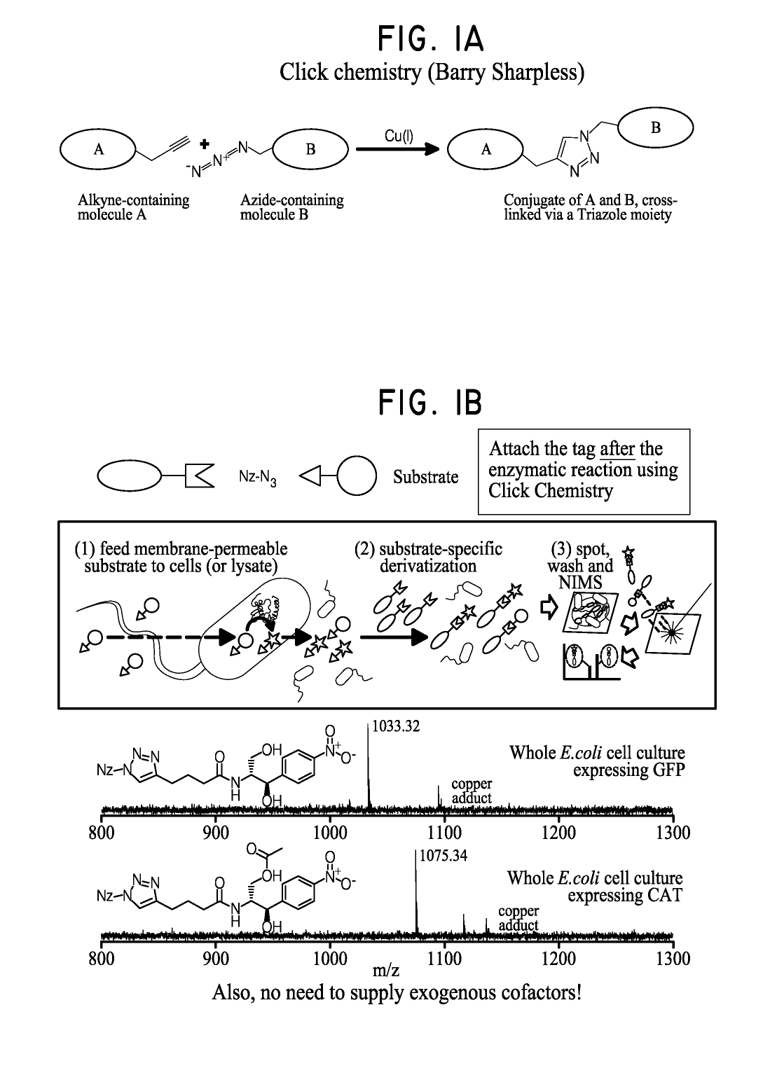

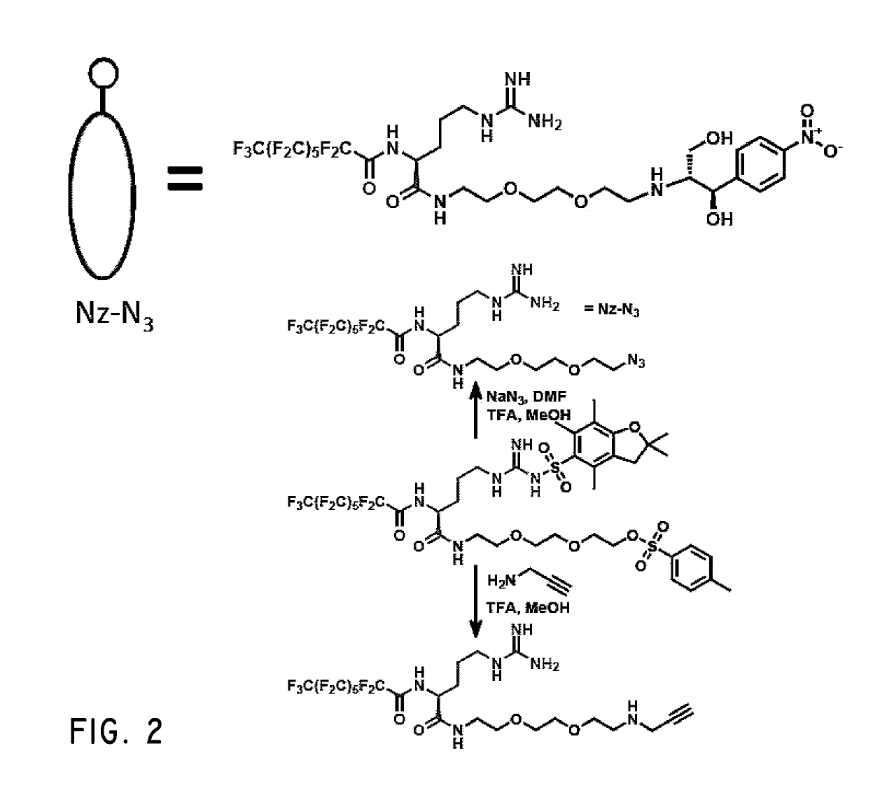

Various techniques can be used to couple the mass probe with the reaction product. For example, the mass probe can be coupled to the reaction product using click chemistry. FIGS. 1A-B illustrate a non-limiting example of tagging of the reaction product using click chemistry. As shown in FIGS. 1A-B, the reaction product and the mass probe can be coupled using the azide alkyne Huisgen cycloaddition using a Copper (Cu) catalyst at room temperature as described by Rostovtsev et at., Angewandte Chemie International Edition 41 (14): 2596-2599 (2002), the content of which is hereby incorporated by reference. As shown in FIG. 1A, an alkyne-containing molecule A can react with an azide-containing molecule B form a conjugate of A-B crosslinked with a triazole moiety. As shown in FIG. 1B, in a Probing Enzyme with “Click”-Augmented NIMS (PECAN) method for detecting chloramphenicol acetyltransferase (CAT) activity, substrates are reacted first and then the reaction products are tagged and after the reaction using click chemistry. PECAN method can be used for probing whole cell enzymes. In PECAN, substrates having an alkyne are nonpolar and can diffuse across the cell membrane for the enzyme to produce reaction products. For example, a fluorous tag is attached to the azide using the synthesis shown in FIG. 2B. Membrane-permeable substrate having an alkyne are fed to cells (or lysate) and the reaction products are released. Substrate-specific derivatization is carried out and the fluorous tag is attached to the reaction products using click chemistry by the azide alkyne Huisgen cycloaddition. The tagged reaction products are spotted, washed and analyzed by NIMS and then identified as shown in the spectra below. The azide can attached to a fluorous probe using the scheme shown in FIGS. 2A-B. The alkyne can be added to the substrate and fed to cells or a crude cell lysate to allow the reaction occur. Using click chemistry, the reaction products can be coupled to the fluorous probe and then analyzed. In some embodiments, the mass tag is coupled to the reaction product via a triazole moiety.

As another non-limiting example, the mass probe can be coupled to the reaction product by an oxime linkage. Without being limited to a particular theory, it is believed that a mass probe having an O-hydroxylamine can be coupled to the reaction product(s) of polysaccharide degradation through chemical condensation of an O-hydroxylamine and an aldehyde to form stable oxime derivative(s).

CAT is a common clinical antibiotic resistance mechanism, and thus there is a need to develop fast, sensitive and reliable method for detecting CAT activity. In some embodiments, the method described herein is used to detect CAT activity in cell lysate or whole cells. The method described herein. In some embodiments, can also be used for screening libraries where CAT is used as a reporter gene in a high-throughput fashion. CAT expression in combination with the method described herein can also be able to measure intracellular acetyl-CoA concentrations, which is frequently an important variable in metabolic engineering projects.

Cytochrome P450s are valued for their ability to perform activation chemistry, and hence they are frequently the subject of directed evolution experiments. Cytochrome P450s also play an important role in human drug metabolism. In some embodiments, the method described herein is used for detecting cytochrome P450 activity.

### Mass Spectrometry

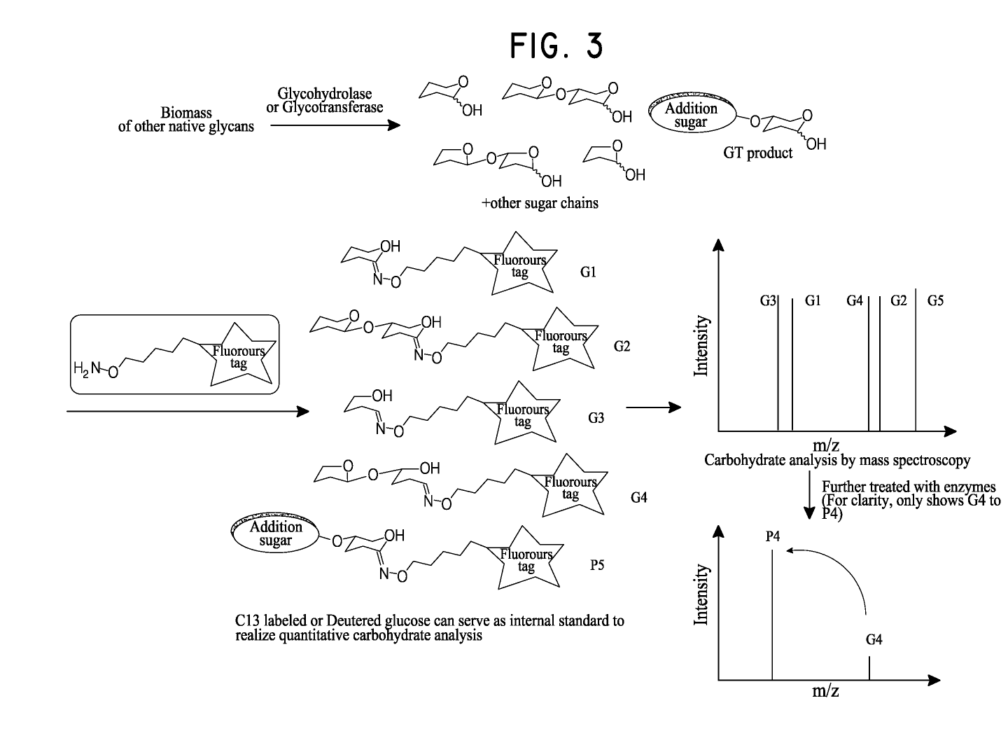

In the methods described herein, analyzing the reaction products can comprise identifying the reaction product, or determining the concentration of the reaction product in the reaction mixture, or both. As described herein, mass spectrometry (MS)-based technologies can be used to perform high-throughput assays for detecting enzymatic activities. Nimzyme is a Nanostructure Initiator Mass Spectrometry (NIMS)-based analytical technique that can detect enzymatic activity in complex mixtures such as crude cell lysate. It circumvents time-intensive chromatographic separations by means of an in situ fluorous affinity purification (FIG. 3). For example, as shown in FIG. 3, an enzyme to be screened can react with a starting crude substrate such as biomass or other native glycans. The reaction products are then tagged with a biomass tag such as a fluorous tag. The reaction products can then be analyzed using a mass spectrometry analysis whereby individual reaction products can be identified by specific peaks due to the biomass tag. In some embodiments, these methods can be used in combination with other methods such as acoustic sample deposition in a high-throughput enzyme activity assay.

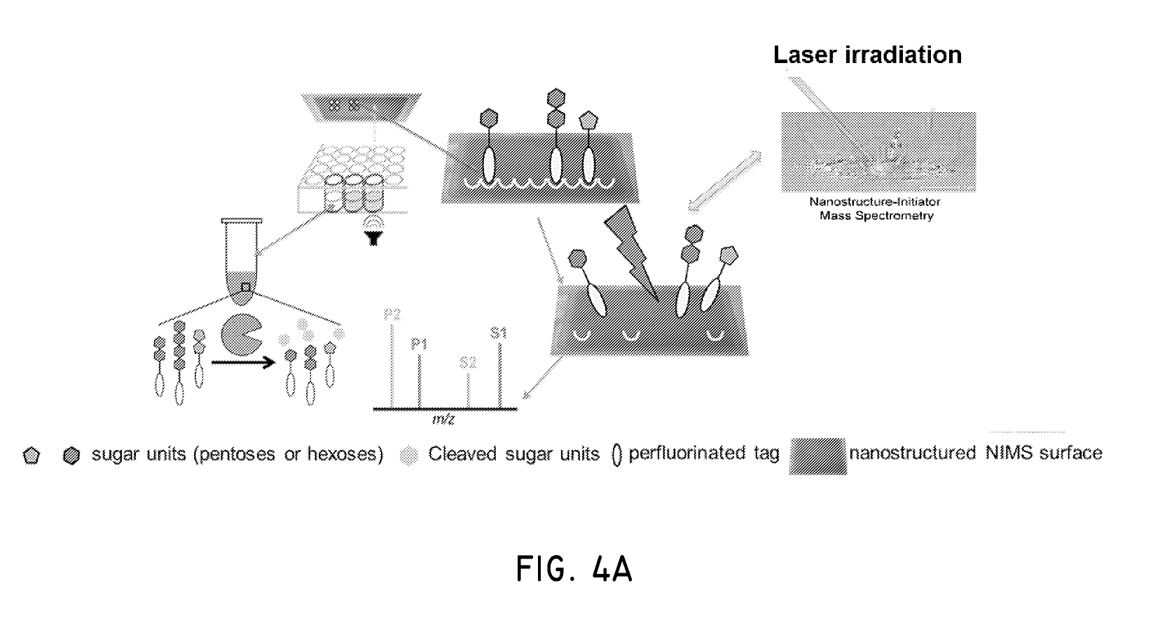

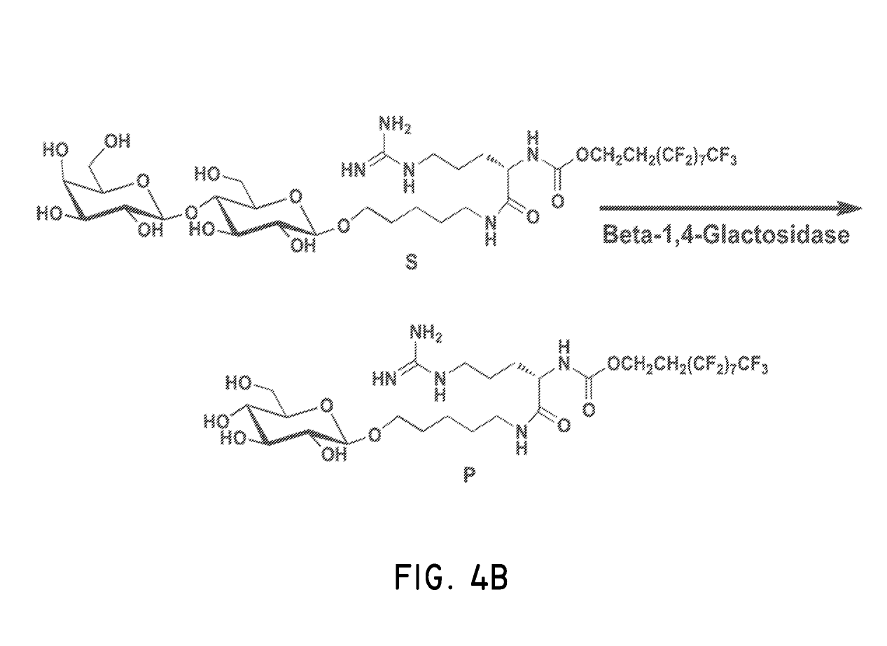

In some embodiments, the mass of the reaction product generated by incubating a sample containing one or more enzyme substrates with one or more enzymes can be determined by mass spectrometry, for example NIMS. NIMS is described in Northen et al., Nature 2007, 449, 1033-1036; Northen et al., Proc. Natl. Acad. Sci. USA 2008, 105, 3678-3683; and U.S. Patent Publication No. 2008/0128608, the contents of which are herein fully incorporated by reference in their entireties. Production of NIMS chips is described in detail in Woo et al., Nat. Protoc. 2008, 3, 1341-1349, the content of which is also herein incorporated by reference in its entirety. An exemplary application of NIMS for detecting the activities of a plurality of enzymes in a multiplexed assay is described in U.S. Patent Publication No. 20120225797, the content of which is hereby incorporated by reference in its entirety. FIG. 4A is a schematic illustration of a non-limiting example of high throughput surface-based enzyme assay, and FIG. 4B shows the reaction of Lactose substrate S incubated with β-1,4-galactosidase to form a reaction product P. As shown in FIG. 4A, in this high throughput surface-based enzyme assay, the reaction mixture was carried out on tagged substrates, the products of the reaction were then transferred to a NIMS chip, for example using acoustic deposition. Product to starting material mass intensity ratio (P/S) is used to determine enzyme activity on NIMS chip.

A variety of apparatuses can be used in NIMS to measure the mass-to-charge ratio of the ionized target. For example, in several embodiments a time-of-flight mass analyzer is used for measuring the desorbed and ionized target. However, other non-limiting examples of mass analyzers that can be used include magnetic ion cyclotron resonance instruments, deflection instruments, and quadrupole mass analyzers.

Acoustic deposition with NIMS is described by some of the inventors and others in Greying et al. In Anal Bioanal Chem. 2012 May; 403(3):707-11. doi: 10.1007/s00216-012-5908-8. Epub 2012 Mar. 10, the content of which is hereby incorporated by reference in its entirety. Acoustic deposition may be possible using other acoustic deposition devices such as the LABCYTE Portrait 630 reagent multi-spotter which is optimized to deposit reagents onto tissue sections for MALDI imaging mass spectrometry. Tagged model substrates enable rapid detection and characterization of enzymatic activities from complex environmental samples and crude cell lysates. Integration of this approach with nanoliter-scale acoustic sample deposition enables rapid profiling of targeted activities. Using this approach, over 60,000 assays were performed to characterize the specificity, temperature and p11 optima from a set of 200 uncharacterized beta-glucosidases. In some embodiments, the reaction products are analyzed by NIMS in conjunction with acoustic deposition.

In some embodiments, the disclosure also provides for systems and processes for the display, imaging, mapping and analysis of the enzymatic activity detected by the methods herein.

### Methods for Detecting Enzymatic Activity

Some embodiments disclosed herein provide a method of detecting the activities of an enzyme sample. The enzyme sample, in some embodiments, comprises multiple enzymes, for example two, three, four, or more enzymes. In some embodiments, the method comprises (a) incubating a substrate sample with an enzyme sample to form a reaction mixture, wherein a substrate sample containing one or more substrates for an enzyme sample, and wherein the reaction mixture contains one or more reaction products; (b) coupling the one or more reaction products with a mass probe to form tagged reaction products; and (c) detecting the enzymatic activity of the enzyme sample by analyzing at least one of the one or more reaction products. The reaction products can be analyzed by MS, for example, NIMS, for detecting the activity of the enzyme sample. The presence and/or amount of the reaction product in the reaction mixture can be determined by, for example, identifying the reaction product ions in the mass spectrum. The activity of the enzyme(s), in some embodiments, correlate with the amount of the reaction product(s) in the reaction mixture. In some embodiments, the activity of the enzyme correlates (e,g., positively correlates) with the amount of the reaction product ions. As described above, NIMS is described, for example, in US Patent Publication No. 20080128608, which is hereby expressly incorporated by reference.

Also disclosed is a method for detecting the activities of a plurality of enzymes in a multiplexed assay. The method, in some embodiments, allows simultaneous detection of the activities of multiple enzymes. For example, the activities of two, three, four, or more enzymes may be detected simultaneously by the method.

In some embodiments, the method includes incubating a substrate sample containing substrates for the plurality of enzymes to obtain reaction products; coupling the reaction products with a mass probe to form tagged reaction products; and detecting activities of the plurality of enzymes by analyzing the reaction products. In some embodiments, analyzing the reaction products is performed using NIMS. Detecting activities of the plurality of enzymes in the sample can be performed by identifying the reaction product ions in a mass spectrum. The activity of the enzymes, in some embodiments, correlate with the amount of the reaction products in the reaction mixture. In some embodiments, the activities of the enzymes correlates (e.g., positively correlates) with the amount of the reaction product ions.

In some embodiments, the reaction product can be applied to a hydrophobic NIMS chip surface for analyzing the reaction product. The hydrophobic NIMS chip surface can, for example, include a perfluorinated coating. In some embodiments, the substrate comprises a substrate head group linked to a perfluorinated tag that forms micelles under aqueous conditions. In some embodiments, the substrate interacts with the NIMS chip surface via fluorous-phase-interactions.

Also disclosed herein is a method for screening enzyme activity. In some embodiments, the method comprises (a) providing a substrate sample; (b) reacting the substrate sample with one or more candidate enzymes to be screened to form reaction products; (c) coupling the reaction products with a mass tag; and (d) identifying and analyzing the reaction products to determine the activity of each candidate enzyme.

In some embodiments, the substrate sample is a heterogeneous reaction mixture that may contain the substrate of the candidate enzyme, including but not limited to, a biomass, crude lysate, cell culture, plant or organic matter, native glycans. The substrate sample can be an environmental sample, a biological sample, or a combination thereof. Non-limiting examples of substrate sample that can be assayed in some embodiments described herein include plant matter, wood, leaves, paper waste, soil, compost, agriculture waste (e.g. livestock waste), mulch, dirt, clay, and garbage.

The methods described herein allows enzyme library screening (e.g., screening for desired hydrolytic enzymes). In screening for desired hydrolytic enzymes (e.g., high performance hydrolytic enzymes), if incomplete hydrolysis is observed upon screening a first enzyme, the mixture can be screened for additional enzymes that would complete the hydrolysis. For example, additional enzyme(s) can be added to the enzyme cocktail until the desired conversion of biomass is achieved. The methods also can be used to develop or optimize enzyme cocktail recipes. Using the methods, compositions and systems described herein, various enzyme cocktails can be screened quickly to, for example, optimize and vary the ratio of enzymes in the cocktail, and the optimal time to add an enzyme to the cocktail until the desired conversion of biomass is achieved. Thus, in some embodiments, the methods can provide for enzyme cocktail optimization by providing; fast, efficient analysis of native glycans using high specificity mass spectrometry based enzyme assays.

Some embodiments provide a method for detecting enzyme activity in a whole cell (e.g., an intact cell or a partially intact cell). In some embodiments, the method comprises: (a) incubating a substrate with a cell to form a mixture, wherein the cell is suspected of containing an enzyme that can interact with the substrate; b) contact the mixture with a mass probe, wherein the mass probe is configured to couple with a reaction product formed by the interaction between the enzyme and the substrate; and (c) detecting the enzyme activity in the cell by analyzing the reaction product. In some embodiments, analyzing the reaction product in step (c) comprises determining the presence or absence of the reaction product in the mixture. In some embodiments, analyzing the reaction product in step (c) comprises determining the concentration and/or amount of the reaction product in the mixture. The amount and/or concentration of reaction product in the mixture can, in some embodiments, correlates (e.g., positively correlates) with the activity of the enzyme in the cell. As described herein, in some embodiments, the substrate can permeate into the cell. In some embodiments, the substrate comprises alkyne. In some embodiments, the substrate comprises azide.

Further described herein is a method for monitoring enzymatic degradation of a substrate sample, for example a biomass. The method, in some embodiments, includes (a) incubating a substrate sample with an enzyme sample to form a reaction mixture, wherein the reaction mixture contains one or more reaction products; (b) coupling the one or inure reaction products with a mass probe to form tagged reaction products; and (c) determining the extent of enzymatic degradation of the substrate sample by analyzing at least one of the one or more reaction products. Steps (b) and (c) can be, in some embodiments, repeated one or more times according to the need for monitoring the enzymatic degradation process. For example, steps (b) and (c) can be repeated according to a pre-determined schedule to monitoring the process of enzymatic degradation of the substrate sample. Time-course of the enzymatic degradation progress can be depicted. During the monitoring, the composition of the enzyme sample can be adjusted, for example, by adding additional enzyme(s) (e.g., one or inure enzymes already in the enzyme sample, or one or more new enzyme) into the enzyme sample, or altering the concentration of the one or more enzymes already in the enzyme sample, and repeat steps (b) and (c) to determine the changes in the enzymatic degradation process. This approach can allow screening of enzyme cocktails with desired enzyme activity profile.

The reaction products can then be analyzed using a mass spectrometry analysis whereby individual reaction products can be identified by specific peaks due to the biomass tag. Suitable analysis methods may include but are not limited to matrix-assisted laser desorption ionization (MALDI), nanoparticle initiator mass spectrometry (NIMS) and secondary ion mass spectrometry (SIMS); Laser Desorption; Desorption ElectroSpray Ionization (DESI); Probe ElectroSpray Ionization (PESI); or Laser Spray. Various instrument modalities may include but are not limited to time-of-flight (TOF), Orbitrap, Fourier-transform ion cyclotron (FTIR), magnetic sector, quadrupole, or other mass spectrometers. In a preferred embodiment, tandem mass spectrometers (MS/MS) are used, such as TOF-TOF or Quadrupole-TOF, wherein the second MS collects fragmentation spectra for molecular characterization of ions analyzed by the first mass spectrometer.

The methods, systems and compositions described herein allows quantitation of reaction products, and thus permits diagnostic, time-dependent analyses of individual enzymes (e.g., glycoside hydrolases) and their combinatorial synergies.

### EXAMPLES

Additional embodiments are disclosed in further detail in the following examples, which are not in any way intended to limit the scope of the claims.

### Example 1

### Screening Glycoside Hydrolases for Thermo-Stability and Substrate Specificity (NIMS-Cellobiose, Maltose, Lactose, Xylobiose)

Glycoside hydrolases (GHs) are a critical class of enzymes for the conversion of plant biomass (cellulose and hemicellulose) into fermentable sugars and therefore central to the development of lignocellulosic biofuels. High throughput activity assays for GHs are needed to support enzyme discovery, engineering and cocktail development. Enzyme assays using nanostructure-initiator mass spectrometry have been developed by using tagged model substrates to enable rapid detection and characterization of GHs activities from complex environmental samples and crude cell lysates. Integration of this approach with nanoliter-scale acoustic sample deposition enables rapid profiling of targeted GH activities. Using this approach over 60,000 assays were performed to characterize the specificity, temperature and pH optima from a set of 200 uncharacterized beta-glucosidases.

### Example 2

### Time-Resolved Catalysis by Mass Spectrometry (MS)

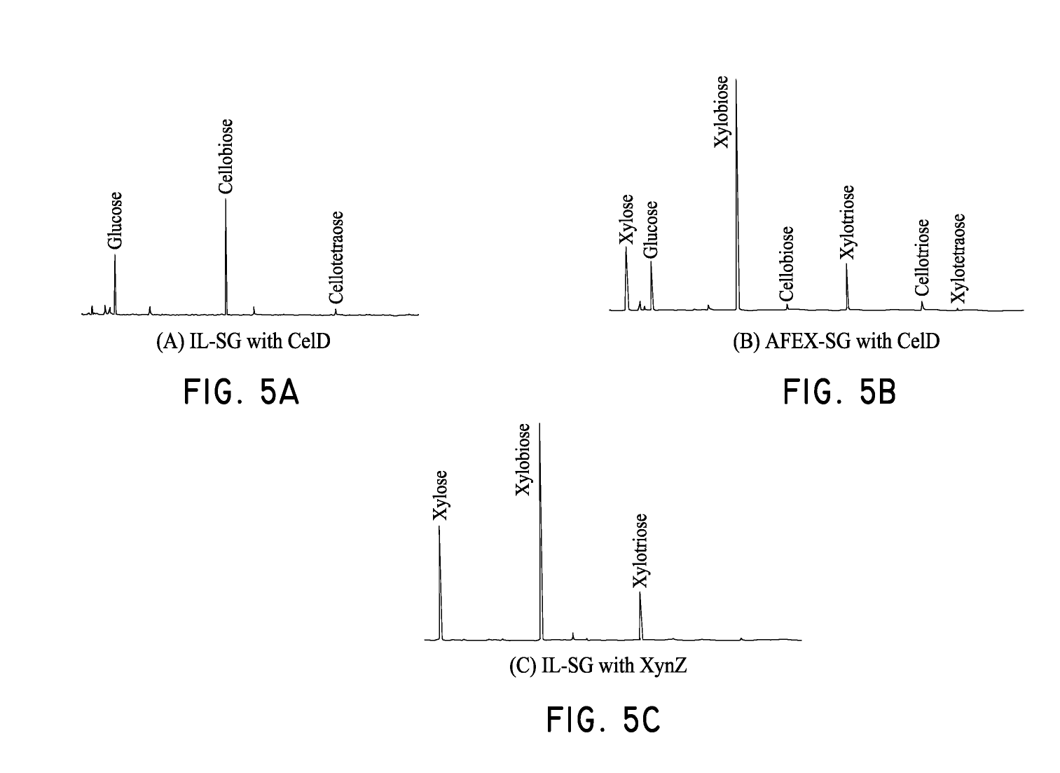

FIGS. 5A-C show the mass spectra of the reaction products by various enzymes with pre-treated switchgrass.

MS methods allow direct detection of the cascades of solubilized products produced by a single enzyme or by enzyme cocktail. FIG. 5A shows distribution (glucose, cellobiose, cellotriose) of reaction products from the reaction of IL-SG switchgrass with enzyme CelD. Analysis of the reaction products of the reaction of switchgrass AFEX-SG with enzyme Cel D also shows C5 sugars: xylose, xylobiose, xylotriose and xylotetraose (FIG. 5B). And FIG. 5C shows that the reaction of IL-SG switchgrass with enzyme XynZ produced xylose, xylobiose and xylotriose.

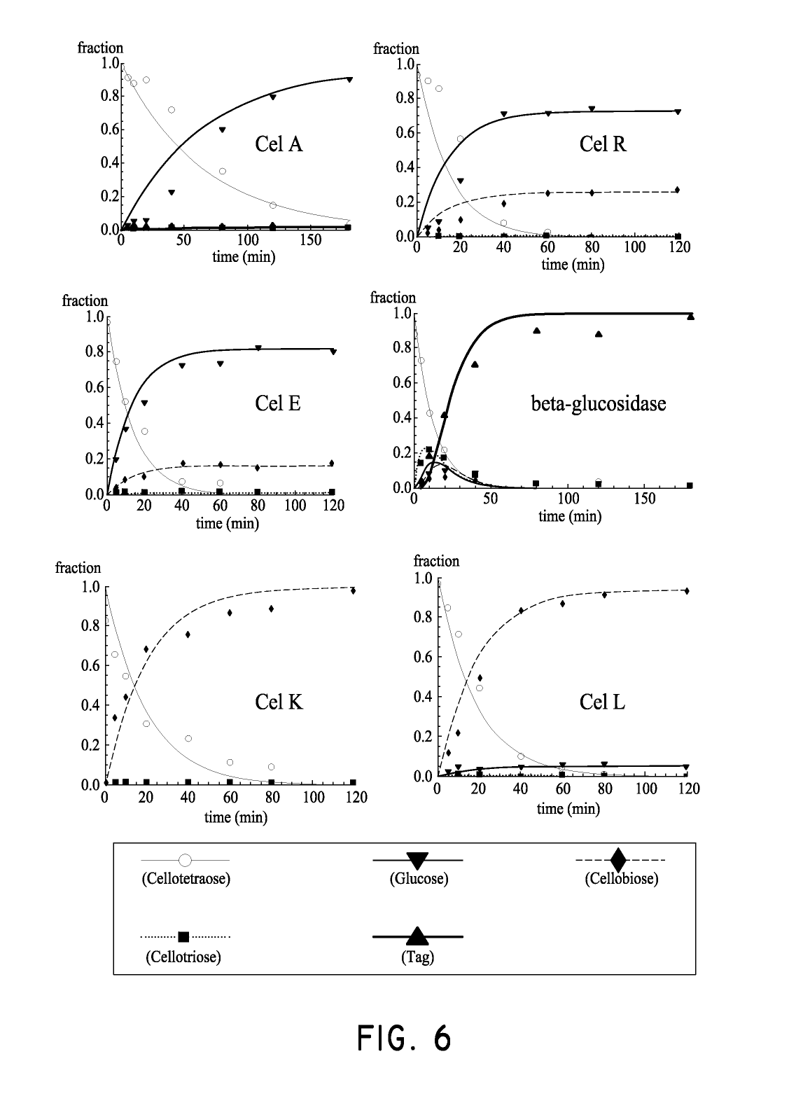

FIG. 6 shows time-resolved catalysis by MS with a diagnostic probe (NIMS-Cellotetraose). Enzymes tested show different reaction time-courses.

### Example 3

### Real Time Monitoring of Biomass Deconstruction Using Oxime-NIMS Method

This example describes a high throughput Oxime-NIMS method to rapidly characterize activities of glycoside hydrolases (GHs) against a range of glycan substrates by combining NIMS analysis. The NIMS analysis was accomplished by using a mass probe that efficiently forms an oxime linkage with the reducing ends of soluble sugars. This probe was added after enzyme hydrolysis, affording both highly efficient modification with the mass-diagnostic tag and subsequent high sensitivity analysis of oligosaccharide mixtures.

These requirements are being overcome using oxime chemistry to attach the substrate directly to native glycans. Here an aminooxy-alkyl functional group, is used to react with the reducing ends of various oligosaccharides from the enzymatic reactions to form oxime. This assay is being used to characterize in vitro expressed C. thermocellum cellulosomal proteins from GLBRC. AFEX and IL pretreated Switchgrass are used as the model substrates to study those enzyme function. Time-dependent cascades of products reveal diagnostic differences for different enzymes.

I. Methods and Materials

All chemicals were purchased as reagent grade and used without further purification. Flash column chromatography steps were performed on a CombiFlash Rf chromatography system from Teledyne ISCO (Lincoln, Neb.). Reactions were monitored using analytical thin-layer chromatography (TLC) in EM silica gel 60 F254 plates and/or by staining with acidic ceric ammonium molybdate or ninhydrin. ¹H NMR and ¹³C NMR spectra were recorded on either a Bruker AV-600 or AVB-400. Chemical shifts (in ppm) were assigned according to the internal standard signal of CDCl₃ (δ=7.26 ppm), CD₃OD (δ=3.31 ppm), or CDCl₃ (δ=77.16 ppm) and CD₃OD (δ=49.00 ppm) for ¹³C NMR. Coupling constants (J) are reported in Hertz, and the splitting patterns are described by using the following abbreviations: s, singlet; d, doublet; dd, doublet of doublets; ddd, doublet of doublet of doublets; t, triplet; dt, doublet of triplets; q, quartet; m, multiple; AB, AB spin system. Infrared (IR) spectra were recorded on a Nicolet MAGNA-IR 850 spectrometer and are reported in frequency of absorption (cm⁻¹). High resolution mass spectral data were obtained from the University of California, Berkeley Mass Spectral Facility.

A. Synthesis O-alkyloxyamine Flourous Tag

The synthesis of O-alkyloxyamine fluorous probe 7 is outlined in Scheme 1. The Mitsunobu reaction between N-hydroxyphthalimide and N-Boc-5-pentanol provided 1. The tert-butyloxycarbonyl (Boc) protecting group on 1 was removed by trifluoroacetic acid (TFA), and the resulting primary amine 2 was coupled to perfluorous tag 5 using N,N′-diisopropylcarbodiimide (DIC)-mediated amide bond formation². The phthalimide protecting group on 6 was cleaved by hydrazine in chloroform to give the desired chemical probe 7.

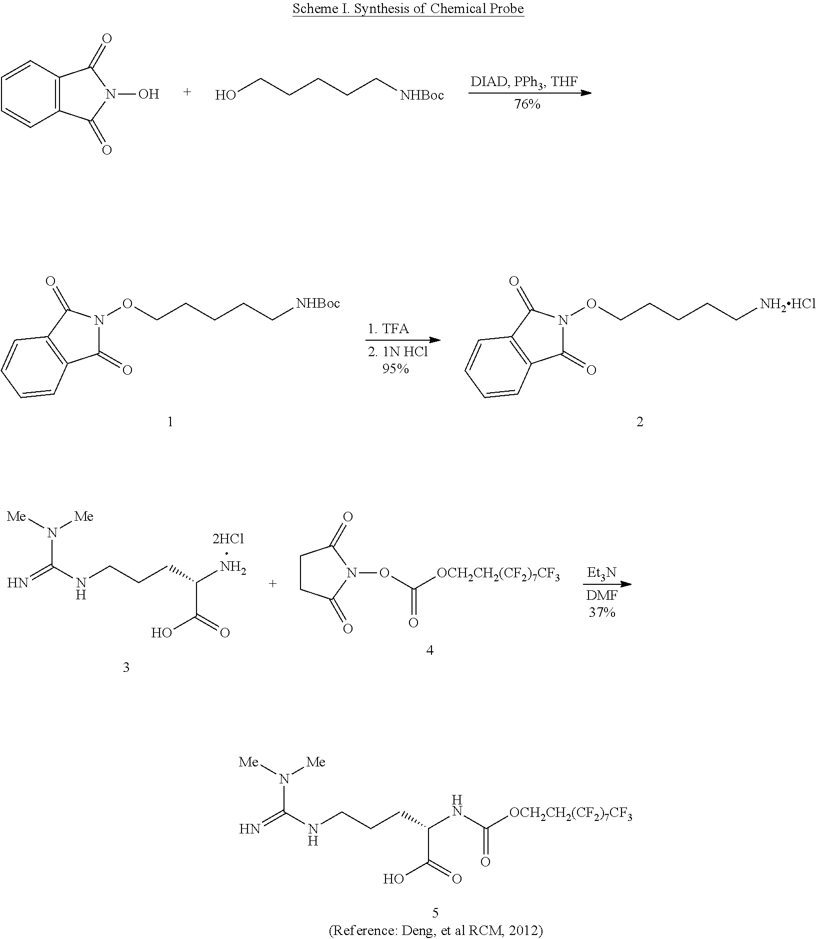

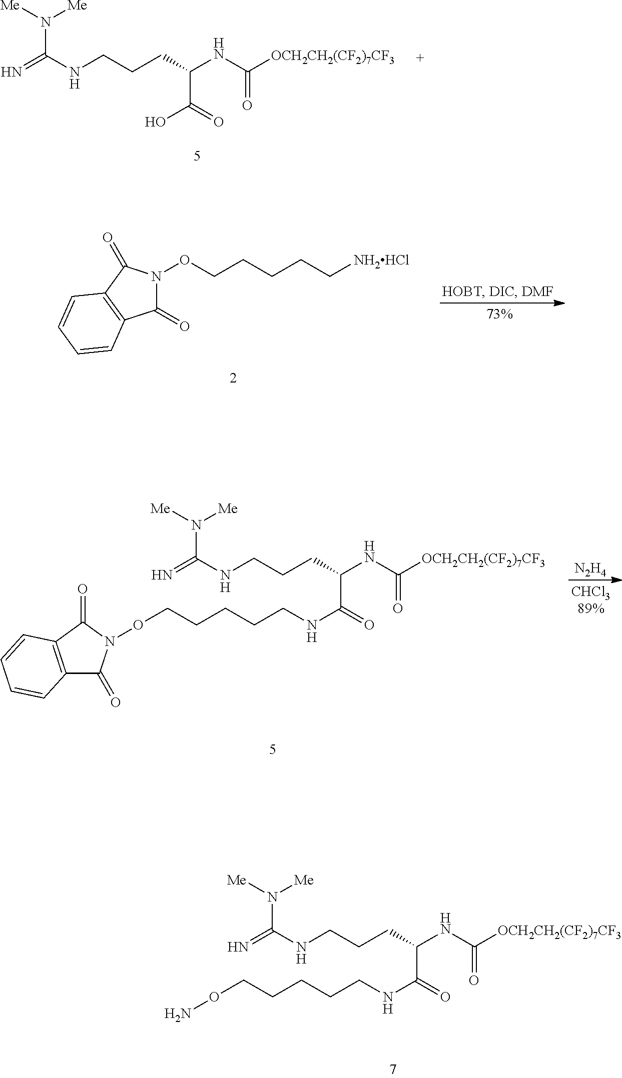

Compound 1. To a stirred solution of 5-(boc-amino)-1-pentanol (1.06 g, 5.21 mmol) and N-hydroxyphthalimide (1.63 g, 9.99 mmol) in THE (30 mL) at 0° C. was added PPh₃ (2.00 g, 7.62 mmol), followed by the drop-wise addition of diethyl azodicarboxylate (1.03 mL, 5.25 mmol). The resulting mixture was stirred at room temperature for 12 h and the solvent was evaporated under reduced pressure. The resulting residue was subjected to column purification to give 1.32 g compound 1 in 76% yield. ¹H NMR (400 MHz, CDCl₃) δ (ppm) 7.85-7.77 (m 2H), 7.76-7.70 (M, 2H), 4.72-4.55 (br. 1H), 4.22-4.15 (t, 2H, J=6.4 Hz), 3.18-3.05 (m, 2H), 1.86-1.72 (m, 2H), 1.60-1.49 (m, 4H), 1.42 (s, 9H). ¹³C NMR (100 MHz, CDCl₃) 163.7, 156.0, 134.5, 128.9, 123.5, 79.0, 78.3, 40.3, 29.6, 28.4, 27.8, 22.9. HRMS (ESI) m/z: Calcd for C₁₈H₂₄N₂O₅, (M+H⁺) 349.1758, found 349.1758.

Compound 2. To a stirred solution of compound 1 (484 mg, 1.39 mmol) in CH₂Cl₂ (1.5 ml) was added trifluoroacetic acid (1.5 mL). The resulting mixture was stirred at room temperature for 1 h and the solvent was evaporated under reduced pressure. The resulting residue was dissolved in 1N HCl (4 mL). After lyophilization, the desired product compound 2 was obtained (380 mg, 95% yield). ¹H NMR (600 MHz, CD₃OD) δ (ppm) 7.81-7.76 (m 4H), 4.20-4.14 (t, 2H, J=6.0 Hz), 3.00-2.94 (t, 2H, J=7.8 Hz), 1.80-1.71 (m, 4H), 1.66-1.59 (m, 2H). ¹³C NMR (150 MHz, CD₃OD) 163.7, 134.5, 128.8, 122.9, 77.5, 39.3, 27.3, 26.7, 22.3. HRMS (ESI) m/z: Calcd for C₁₃H₁₆N₂O₃ (M+H⁺) 249.1234, found 249.1234.

Compound 6. To a stirred solution of compound 5 (prepared according to procedure described in Deng et al., Rapid Commun. Mass Sp., 26:611-615 (2012)) (84 mg, 0.12 mmol) and compound 2 (35 mg, 0.12 mmol) in DMF (6 mL) was added HOBt (22 mg, 0.14 followed by the addition of N,N′-Diisopropylcarbodiimide (22 μL, 0.14 mmol). The resulting mixture was stirred at room temperature for 12 h and the solvent was evaporated under reduced pressure. The resulting residue was subjected to column purification to give 80 mg compound 6 in 73% yield. ¹H NMR (600 MHz, CD₃OD) δ (ppm) 4.44-4.33 (m 2H), 4.10-4.05 (dd, 1H, J=7.8, 6.0 Hz), 3.66-3.61 (t, 2H, J=6.0 Hz), 3.28-3.22 (m, 2H), 3.22-3.16 (t, 2H, J=7.2 Hz), 3.03 (s, 6H), 2.64-2.54 (m, 2H), 1.85-1.75 (m, 1H), 1.73-1.61 (m, 3H), 1.61-1.56 (m, 2H), 1.56-1.49 (m, 2H), 1.40-1.34 (m, 2H). ¹³C NMR (150 MHz, CD₃OD) 172.9, 163.7, 156.37, 156.26, 134.4, 128.8, 122.9, 77.7, 56.7, 54.8, 48.4, 41.5, 38.7, 37.2, 28.5, 27.4, 27.2, 25.0, 22.7. HRMS (ESI) m/z: Calcd for C₃₂H₃₅F₁₇N₆O₆ (M+H⁺) 923.2419, found 923.2415.

Compound 7. To a stirred solution of compound 6 (51 mg, 0.055 mmol) in CHCl₃ (3.0 mL) was added N₂H₄.H₂O (30 μL, 0.62 mmol). The resulting mixture was stirred at room temperature for 1 h and the solid coming out the solution was filtered. The filtrate was evaporated under reduced pressure to give Compound 7 (39 mg, 89% yield). ¹H NMR (600 MHz, CD₃OD) δ (ppm) 4.44-4.33 (m 2H), 4.10-4.05 (dd, 1H, J=7.8, 6.0 Hz), 3.66-3.61 (t, 2H, J=6.0 Hz), 3.28-3.22 (m, 2H), 3.22-3.16 (t, 2H, J=7.2 Hz), 3.03 (s, 6H), 2.64-2.54 (m, 2H), 1.85-1.75 (m, 1H), 1.73-1.61 (m, 3H), 1.61-1.56 (m, 2H), 1.56-1.49 (m, 2H), 1.40-1.34 (m, 2H). ¹³C NMR (150 MHz, CD₃OD) 172.7, 156.37, 156.30, 75.3, 56.7, 54.7, 48.1, 41.5, 38.8, 37.1, 29.0, 28.7, 27.6, 25.0, 22.9. HRMS (ESI) m/z: Calcd for C₂₄H₃₃F₁₇N₆O₄ (M+H⁺) 793.2365, found 793.2378

B. Enzymes

The gene loci of the enzymes used are Cthe_{—}0536 (CelB), Cthe_{—}0912 (XynY) and Cthe_{—}0797 (CelEcc_CBM3a). Additional information on these genes can be found at Uniprot (Apweiler, et al., Nucleic Acids Res, 39: D214-D219 (2011)). All genes were prepared by PCR using Clostridium thermocellum ATCC 27405 genomic DNA as template. The nucleotide sequences encoding the translocation signal peptides predicted by SignalP v4.0⁵ were removed by primer design (as described in Takasuka, et al., Cell-free Translation of Biofuels Enzymes. Methods in Molecular Biology, The Humana Press Inc., Totowa, N.J., 2013), as were the nucleotide sequences encoding the dockerin and esterase domains in XynY, and the dockerin, lipase and GDSL domains in CelE. The truncated CelE gene was fused to the sequence encoding the carbohydrate binding domain 3a (CBM3a) with specificity for crystalline cellulose from Cthe_{—}3077 (CipA). Enzymes were produced using robotic cell-free translation (as described in Takasuka, et al., 2013). The total translation mixture was concentrated ˜3× using a spin concentrator, and then used in assays without further purification. Enzyme concentrations in the total translation mixture were estimated by the Biorad Stain-free gel system (Hercules City, Calif.).

C. Optimization of Coupling Conditions

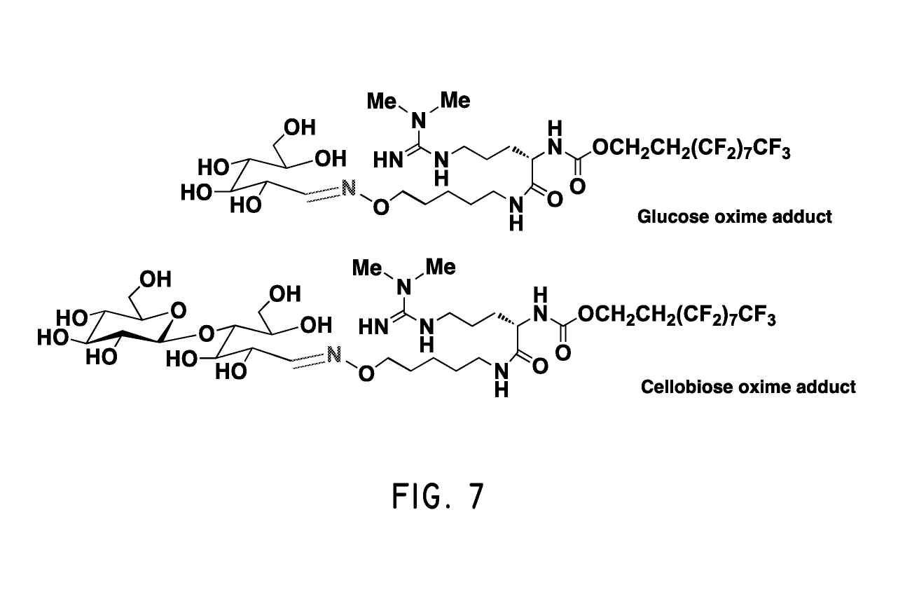

Coupling conditions for reaction of the O-alkyloxyl amine containing fluorous tag were tested over a range of pH and temperature as shown in Table 1, 2 and FIG. 7. HPLC was used to determine reaction yields based on direct detection of the unreacted cellobiose across 6 conditions. These analyses established that the coupling is robust, with only ˜5% difference in yield between the best (condition A) and worst reactions (condition E). The potential for differential reactivity of the O-alkyloxylamine probe with different reducing sugars was tested using HPLC and condition A (Table 2). Results show that 90% of pure pentose glycans (xylose, xylobiose, xylotriose, xylotetraose) and pure hexose glycans (glucose, cellobiose, cellotriose and cellotetraose) were recovered under the optimal experimental conditions. The tagging efficiency was independent of substrate concentration over the desired characterization range (50 μM to 2 mM) for all glycans tested. The overall yield may be due to a thermodynamic equilibrium at the final concentration of tag used due to the relatively small driving force for the coupling reaction (breakage of a C═O bond and 2 H—N bonds, formation of a C═N and 2 O—H bonds, ΔH=−6 kcal/mole). Relative to the cost of carrying out the assays with 10-fold higher concentration of O-alkyloxylamine tag, the selected conditions provide sufficient sensitivity while also supporting high throughput operations.

TABLE 1
Coupling conditions.

| Entry | Substrate^{a} | Buffer pH | Temperature and time | Yields |
|---|---|---|---|---|
| A | Cellobiose | 1.3 | 22° C., 16 h | 90% |
| B | Cellobiose | 2.6 | 22° C., 16 h | 90% |
| C | Cellobiose | 3.0 | 22° C., 16 h | 89% |
| D | Cellobiose | 3.5 | 22° C., 16 h | 87% |
| E | Cellobiose | 4.0 | 22° C., 16 h | 85% |
| F | Cellobiose | 1.3 | 55° C., 5 h | 88% |

^{a}Cellobiose was used as the substrate for testing coupling reaction conditions because it is a disaccharide, and it is readily available in a highly pure form. Under optimized coupling conditions, longer oligosaccharides were found to have similar coupling efficiency, as shown in Table 2.

TABLE 2
Reactivity of different oligosaccharides.

| | Conversion to oxime |
|---|---|
| Glycans | adducts |
| Xylose | 88% |
| Xylobiose | 92% |
| Xylotriose | 94% |
| Xylotetraose | 90% |
| Glucose | 96% |
| Cellobiose | 90% |
| Cellotriose | 87% |
| Cellotetraose | 88% |

D. Reactions with Enzymes Produced by Cell-Free Translation

Cell-free translation was used to produce enzymes for this study with yields of translated enzyme ranging from 0.1-2 mg per mL of reaction mixture. Since the wheat germ extract has no intrinsic hydrolytic reactivity with biomass, additional purifications of the translated enzymes were not necessary. The translation reactions were concentrated by ˜3× to allow smaller volumes to be added to the assays.

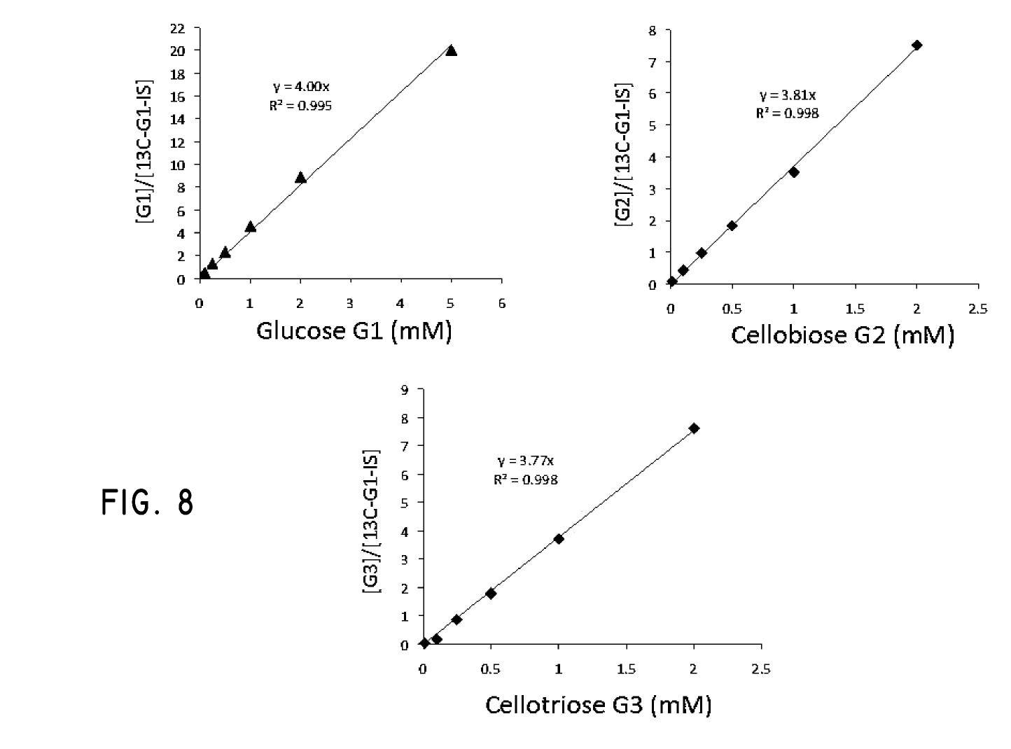

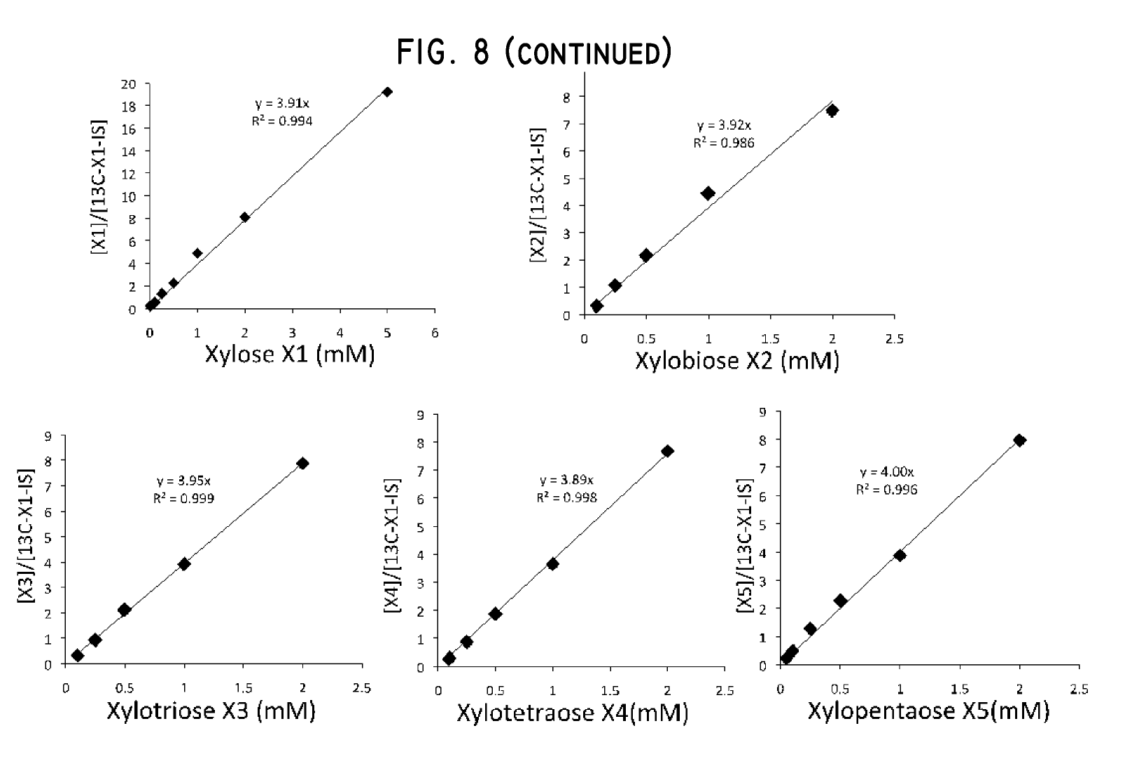

Calibration curves for reaction of oligosaccharides using reaction condition A from Table 1 are shown in FIG. 8. As shown in Table 3, the oxime derivatization method worked well in the presence of the cell-free translation mixture, and signal-to-noise ratios for products were typically greater than 100 for the targeted substrate concentrations.

TABLE 3
Signal to noise ratio for product (e.g. cellobiose oxime adduct)

| Entry | Substrate | Concentration (mM) | S/N |
|---|---|---|---|
| 1 | Cellobiose | 0.01 | 27 |
| 2 | Cellobiose | 0.1 | 152 |
| 3 | Cellobiose | 0.25 | 307 |
| 4 | Cellobiose | 0.5 | 1297 |
| 5 | Cellobiose | 1 | 1378 |
| 6 | Cellobiose | 2 | 2541 |
| 7 | Cellobiose | 5 | 4526 |

E. Enzyme Assays and Conversion of Products

The reactions of enzymes with biomass were carried out in 50 mM phosphate, pH 6.0, with biomass (either IL-SG or AFEX-SG) loading of 10 mg/mL. The reaction was carried out at 60° C. for up to 96 h. The enzymes concentrations of stock solution used were: CelB (1 mg/mL); XynY (0.4 mg/mL); CelEec_CBM3a (20 mg/mL). Details about enzyme assay setup were described in supporting information. Samples were taken at 1, 2, 4, 8, and 24 h. At these times, a 2 μL aliquot of the reaction mixture was transferred into a vial containing 6 μL of 100 mM glycine acetate, pH 1.2, 0.5 μL of a 5.0 mM aqueous solution of [U]-13C glucose, 2 μL of CH3CN, 1 μμL of MeOH, 1 μL of solution probe (100 mM in 1:1 (v/v) H2O:MeOH), and 0.12 μL of aniline. The mixture was incubated at room temperature for 16 h. Test reactions of either CelEcc_CBM3a or XynY (10 μg enzyme) with birch xylan and arabinoxylan (Megazyme, Ireland), were carried out at 55° C. In 100 μL of 50 mM phosphate, pH 6.0, containing 20 mg/ml. of the suspended substrate. Total sugar hydrolysis was monitored by DNS assay after 20 h. Arabinose and xylose were independently monitored using detection kits from Megazyme.

F. Enzyme Kinetic Analyses

A velocity versus substrate concentration analysis was used to determine the kinetic parameters for β-glucosidase (Aa-βG) from the thermoacidophilic bacterium Alicycyclobacillus acidocaldarius 22 using pure cellobiose as substrate. The experimental data were analyzed using the NonlinearModelFit routine of Mathematic v.8 (Wolfram Research) and v=kcat [S]/(KM+[S]) (kcat, KM, and [S] have their usual definitions).

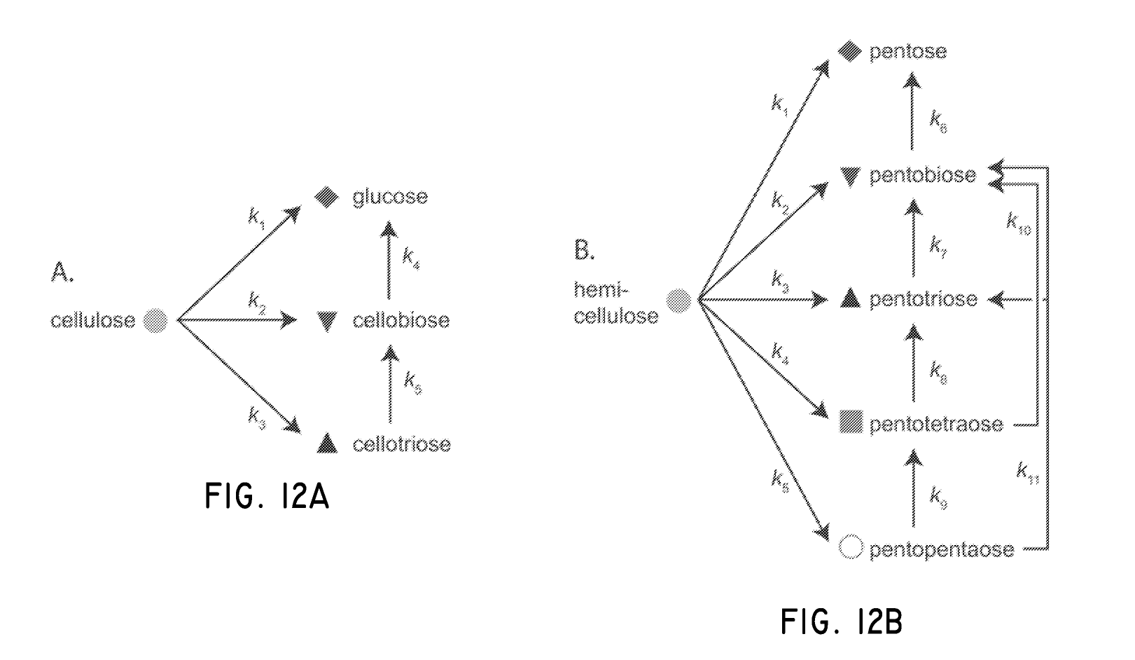

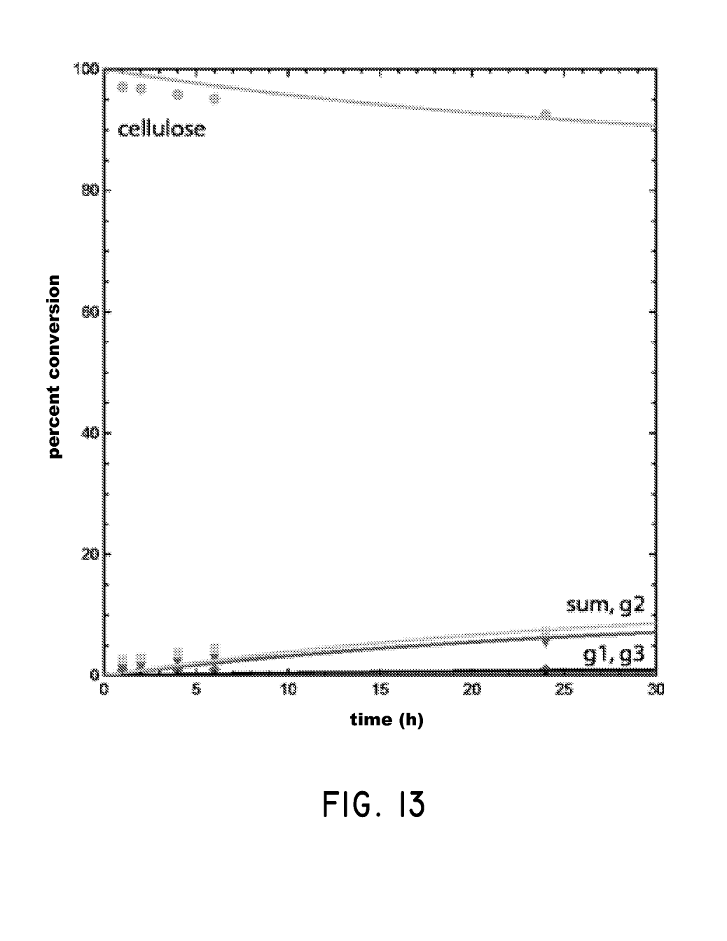

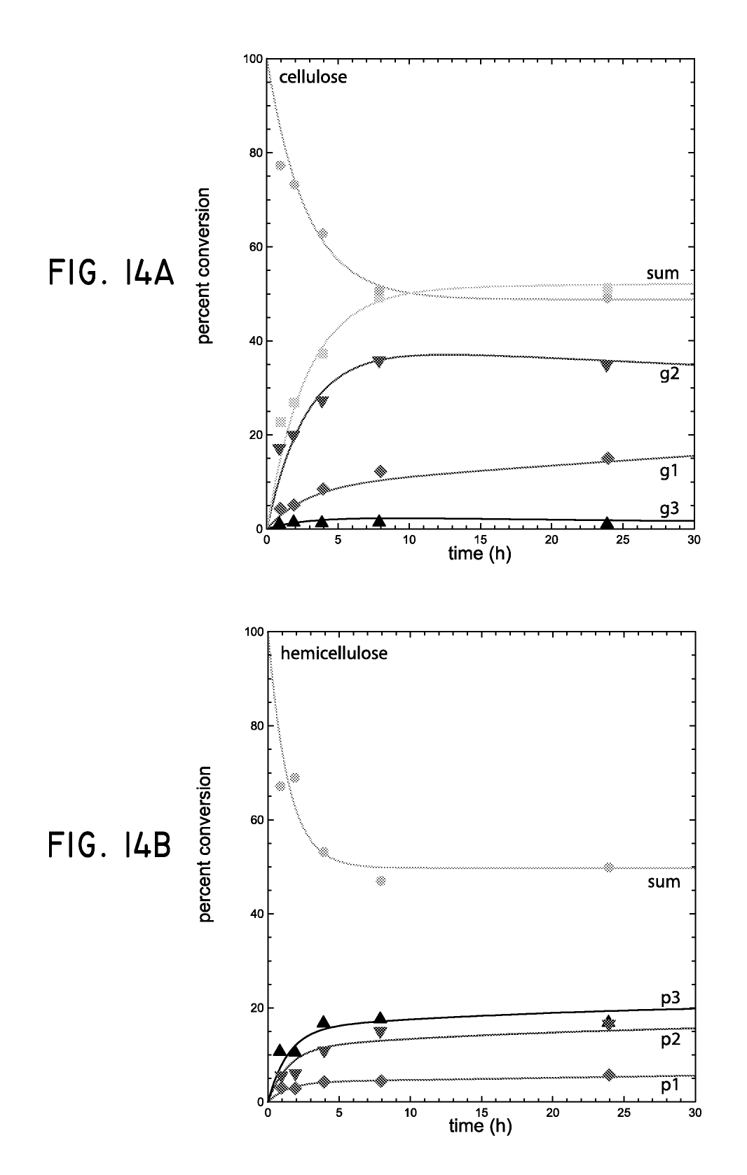

The time dependence of product formation were analyzed by numerical integration using Mathematica v.8 routine NDSolve (differential equations are found in the Supplementary Material) and the Nelder-Mead simplex method for constrained minimization 36. The differential equations incorporate release of soluble oligosaccharides from biomass and their subsequent hydrolysis to end products according to the kinetic schemes shown in FIG. 12. After initial guesses for kinetic constants were established by visual examination of the match between experiment and one-step calculation, successive rounds of parameter optimization with adjustment of parameter constraints were carried out until the sum of the squares difference between calculated and experimental values reached a minimum.

G. Nanostructure Initiator Mass Spectrometry (NIMS)

In each case, 0.12 μL, of the quenched reaction sample was spotted onto the NIMS surface and removed after 30 s. A grid drawn manually on the NIMS chip using a diamond-tip scribe helped with spotting and identification of sample spots in the spectrometer. Chips were loaded using a modified standard MALDI plate. NIMS analysis was performed using a 4800 MALDI TOF/TOF mass analyzer from Applied Biosystems (Foster City, Calif.). Signal intensities were identified for the ions of the tagging products and ˜1000 laser shots were collected. Enzyme activities were determined by measuring the concentration of glycan products using either [U]-13C glucose or [U]-13C xylose as an internal standard.

II. Results

A. Optimization of Coupling Conditions

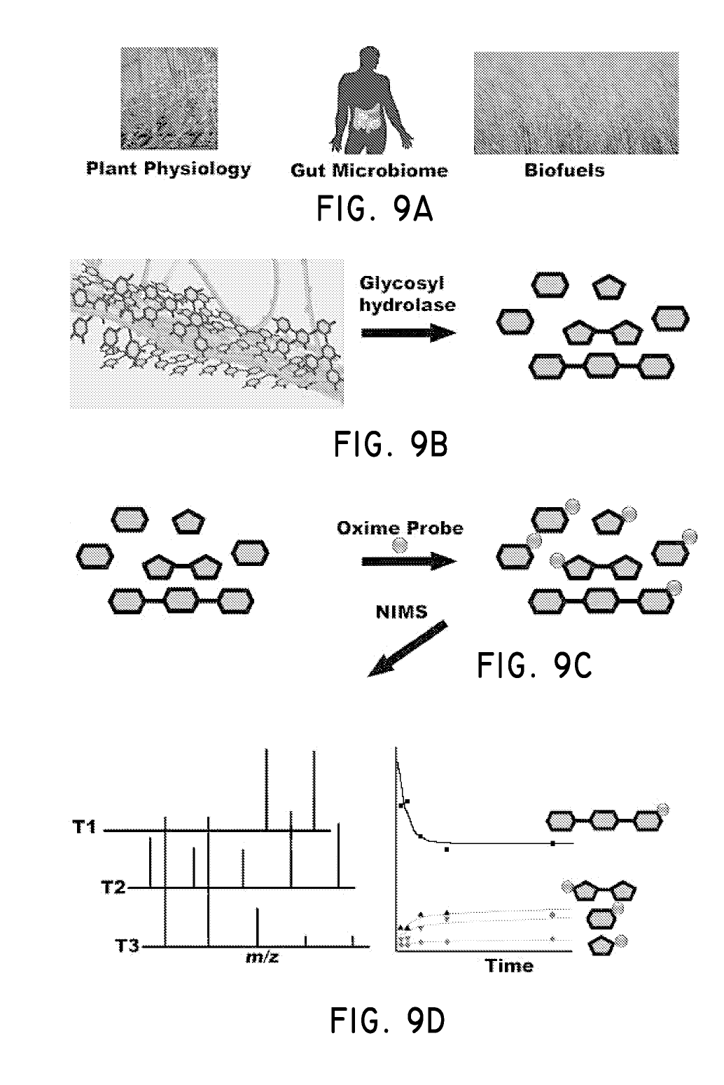

FIGS. 9A-D provide a summary of the overall method presented in this example. Reactions of cellulases and hemicellulases with plant biomass were studied and the favorable chemical condensation of an O-hydroxylamine with an aldehyde to form a stable oxime derivative was utilized. As described herein, combination of the O-hydroxylamine with the perfluorous NIMS tag affords a mass-diagnostic probe with unique utility. Coupling conditions for reaction of the O-hydroxylamine fluorous probe were tested over a range of pH and temperature as shown in Tables 1 and 2, and FIG. 8. HPLC was used to determine reaction yields based on direct detection of the unreacted cellobiose across 6 conditions. These analyses established that the coupling is robust, with only ˜5% difference in yield between the best (condition A) and worst reactions (condition F).

B. NIMS Quantification Using 13C-Labelled Internal Standards

[U]-¹³C labeled monosaccharides (glucose and/or xylose) were used as internal standards (see FIG. 8 for calibration curves). The ratio of the mass spectral intensity of the derivatized glycan of interest at various concentrations relative to a fixed concentration of the labeled, similarly derivatized monosaccharide (0.25 mM) was used to make calibration curves. These curves were linear (r² values of 0.98-0.999) over the desired substrate concentrations (50 μM to 2 mM) for each glycan tested. The slope of the calibration curves for an oligosaccharide represents a combination of the tagging and desorption/ionization efficiencies of the different glycan-probe adducts, and differs for each oligosaccharide.

C. Use of Oxime Tagging for Determination of Kinetic Constants

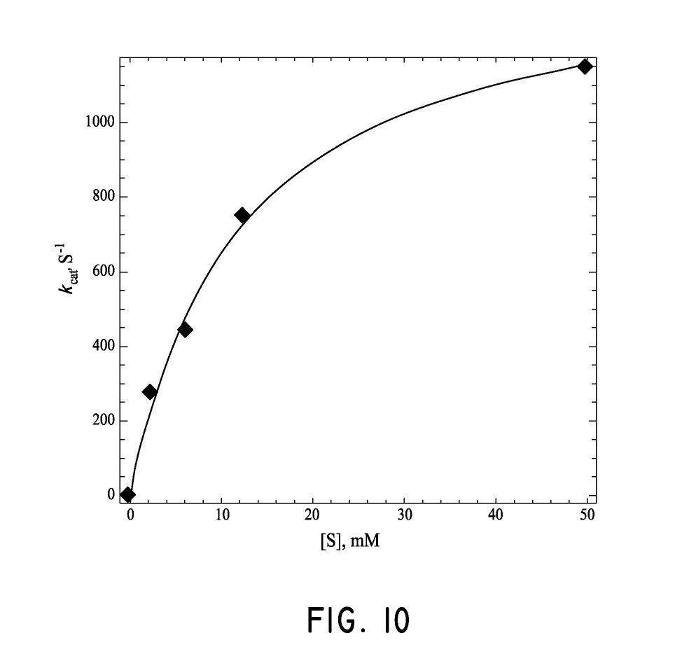

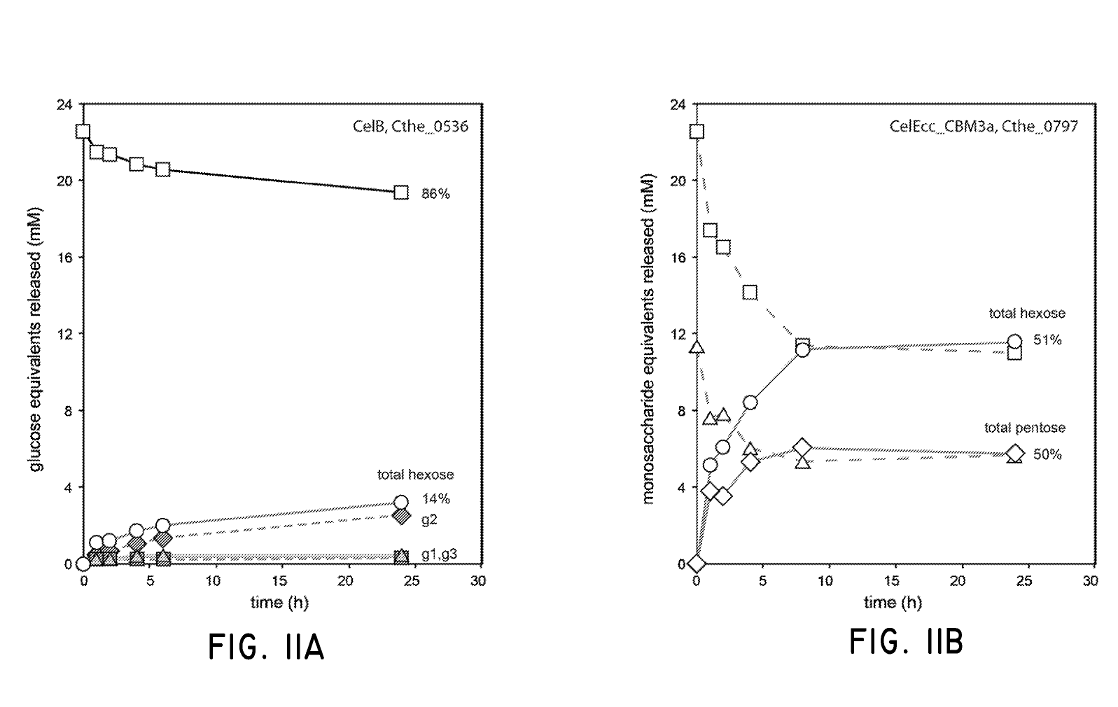

Glucose formed at different time points from the reaction of Alicycyclobacillus acidocaldarius β-glucosidase with varied cellobiose concentration was quantified and used for steady-state kinetic analysis. The data were analysed using the NonlinearModelFit routine of Mathematic v.8 and v=k_{cat} [S]/(K_{M}+[S]), where k_{cat}, K_{M}, and [S] have their usual definitions. The results are shown in FIG. 10. As shown in FIG. 10, the calculated k_{cat} and K_{M}-values of 1430±850 s⁻¹ and 12.1±4.6 mM, respectively, with r²=0.997 and parameter variance estimates given at the 95% confidence level were in excellent agreement with previously reported values (k_{cat}=−1300 s⁻¹; K_{M}=˜10 mM, lacking error analysis), demonstrating the efficacy of the NIMS-based quantification method.

D. Time-Dependent Monitoring of Plant Biomass Deconstruction by Oxime Capture Reveals Differences in Performance and Specificities of Monofunctional and Multifunctional GH Enzymes

One non-limiting exemplary application for the methods disclosed herein is the characterization of GH enzymes used in the conversion of plant biomass to biofuels. In a traditional industrial process, a complex cocktail of dozens of GH enzymes is used at 3-30% enzyme/biomass (30-300 mg/g) loading. The cost to produce this diversity and amount of enzymes is a major challenge to the successful implementation of a biomass-based biofuels industry. GH enzymes that catalyze hydrolysis of multiple types of glycosidic bonds have the potential to reduce the complexity and therefore the cost of these enzyme cocktails by catalyzing hydrolysis of multiple types of glycosidic bonds.

In this example, the oxime-NIMS method was used to detect the reducing sugars produced from enzymatic hydrolysis of ionic liquid treated switchgrass, IL-SG, a relevant pre-treated biofuel crop. Three classes of enzymes from Clostridium thermocellum were included in these studies, including: (1) CelB (Cthe_{—}0549), an endoglucanase that cleaves internal bonds of cellulose only; (2) XynY (Cthe_{—}0912), a potentially processive hemicellulase that consists of CBM22, GH10 and CBM22 domains, and (3) CelEcc_CBM3a, an engineered enzyme that contains the GH5 catalytic domain (Cthe_{—}0797) fused to a cellulose binding module. CelEcc_CBM3a is a multifunctional enzyme that can react with pure polysaccharides such as cellulose, xylan, lichenin, mannan, galactomannan and xyloglucan. Low enzyme loadings of 0.1-2.0% enzyme/g IL-SG (1-20 mg/g) were used to react with IL-SG and time-series data were collected to enable numerical analysis of enzyme specificity and product formation kinetics. FIGS. 11A-B show the time-dependent appearance of total products for CelB and CelEcc_CBM3a based on the capability of the NIMS method to simultaneously detect and quantify hexose and pentose product cascades. As shown in FIG. 11A, CelB reaction gives 14% conversion of the glucan fraction, with cellobiose (g2, green dotted line) representing the major product. As shown in FIG. 11B, CelEcc_CBM3a reaction gives simultaneous ˜50% conversion of the cellulose and hemicellose fractions.

This example further demonstrated that the oxime-NIMS method can also be used to evaluate the potential of multifunctional enzymes.

E. Kinetic Model for Biomass Deconstruction

FIGS. 12A-B show kinetic models for hydrolysis of the cellulose (FIG. 12A) and hemicellulose (FIG. 12B) present in IL-SG into a cascade of solubilized products and also subsequent hydrolysis of the solubilized oligosaccharides.

Differential equations corresponding to the kinetic scheme shown in FIG. 12A for reaction of cellulose.

y[1]=cellulose

y[2]=glucose

y[3]=cellobiose

y[4]=cellotriose

Differential equations

dy[1]/d[t]=−(k1+k2 +k3)y[1][t]

dy[2]/d[t]=k1y[1][t]+k4y[3][t]+k5y[4][t]

dy[3]/d[t]=k2y[1][t]+k5y[4][t]−k4y[3][t]

dy[4]/d[t]=k3y[1][t]−k5y[4][t]

Differential equations corresponding to the kinetic scheme shown in FIG. 12B for reaction of cellulose.

y[1]=hemicellulose

y[2]=pentose

y[3]=pentobiose

y[4]=pentotriose

y[5]=pentotetraose

y[6]=pentopentaose

Differential equations

dy[1]/d[t]=−(k1+k2+k3+k4+k5)y[1][t]

dy[2]/d[t]=k1y[1][t]+2k6y[3][t]+k7y[4][t]+k8y[5][t]+k9y[6][t]

dy[3]/d[t]=k2y[1][t]+2k10y[5][t]+k11y[6][t]+k7y[4][t]−k6y[3][t]

dy[4]/d[t]=k3y[1][t]+k8y[5][t]+k11y[6][t]−k7y[4][t].

dy[5]/d[t]=k4y[1][t]+k9y[6][t]−(k8+k10)y[5][t]

dy[6]/d[t]=k5y[1][t]−(k9+k11)y[6][t]

F. Monofunctional GH5 CelB Gave a Low Yield of Cellulose Products Only

In the reaction of CelB with IL-SG, glucose (g1), cellobiose (g2), and cellotriose (g3) were observed (FIG. 11A), with cellobiose accumulating as the major product (k₂=0.019 h⁻¹). In total, the three hexose products accounted for ˜15% conversion of the total cellulose present in IL-SG. The low yield of solubilized sugar is consistent with the known function of CelB as a non-processive endoglucanase.

FIG. 13 shows the time course analysis for formation of individual products during the reaction of CelB with IL-SG. A, time course for formation of hexose products: cellulose fraction in unreacted biomass (green solid circles); sum of products (cyan squares); cellobiose (g2, purple down triangles); glucose (g1, blue diamonds); cellotriose (g3, black up triangles). Solid lines were generated by solving differential equations corresponding to the kinetic scheme of FIG. 12A.

Table 4 shows the apparent rates for product formation modeled according to the kinetic scheme of FIG. 12A and normalized to the amount enzyme active site present.

TABLE 4
Apparent rates (h⁻¹)^{a} in reactions with IL-SG according
to the kinetic schemes of Figures 12A-B.

| | | cellulose^{b} | CelEcc_CBM3a + | hemicellulose | | CelEcc_CBM3a + |
|---|---|---|---|---|---|---|
| Rate | CelB | CelEcc_CBM3a | XynY^{c} | CelEcc_CBM3a | XynY | XynY^{c} |
| k₁ | 0.001 | 0.022 | 0.015 | 0.020 | 0.102 | 0.103 |
| k₂ | 0.019 | 0.089 | 0.115 | 0.054 | 0.301 | 0.321 |
| k₃ | 0.003 | 0.006 | 0.037 | 0.073 | 0.160 | 0.196 |
| k₄ | 0.000 | 0.002 | 0.002 | 0.059 | 0.102 | 0.150 |
| k₅ | 0.001 | 0.005 | 0.015 | 0.026 | 0.026 | 0.035 |
| k₆ | — | — | — | 0.000 | 0.000 | 0.000 |
| k₇ | — | — | — | 0.000 | 0.006 | 0.015 |
| k₈ | — | — | — | 0.000 | 0.000 | 0.000 |
| k₉ | — | — | — | 0.000 | 0.000 | 0.005 |
| k₁₀ | — | — | — | 0.000 | 0.000 | 0.000 |
| k₁₁ | — | — | — | 0.013 | 0.000 | 0.000 |

^{a}Rates were normalized to the nmol of enzyme active sites; XynY was used to normalized the reactions of CelEcc_CBM3a + XynY.
^{b}Rates k₆-k₁₁ are not included in the kinetic scheme (Figure 12) for cellulose reaction.
^{c}Rates determined for CelEcc_CBM3a + XynY represent the sum of products observed from both enzymes.

G. Multifunctional GH Enzyme Hydrolyzes Both Cellulose and Hemicellulose.

FIG. 11B shows that engineered CelEcc_CBM3a (20 mg/g biomass, 2.0% enzyme/biomass loading) hydrolyzed both the cellulose and hemicellulose present in biomass to give ˜50% of the expected solubilized yield of both hexoses and pentoses.

H. Analysis of Cellulose Hydrolysis Kinetics.

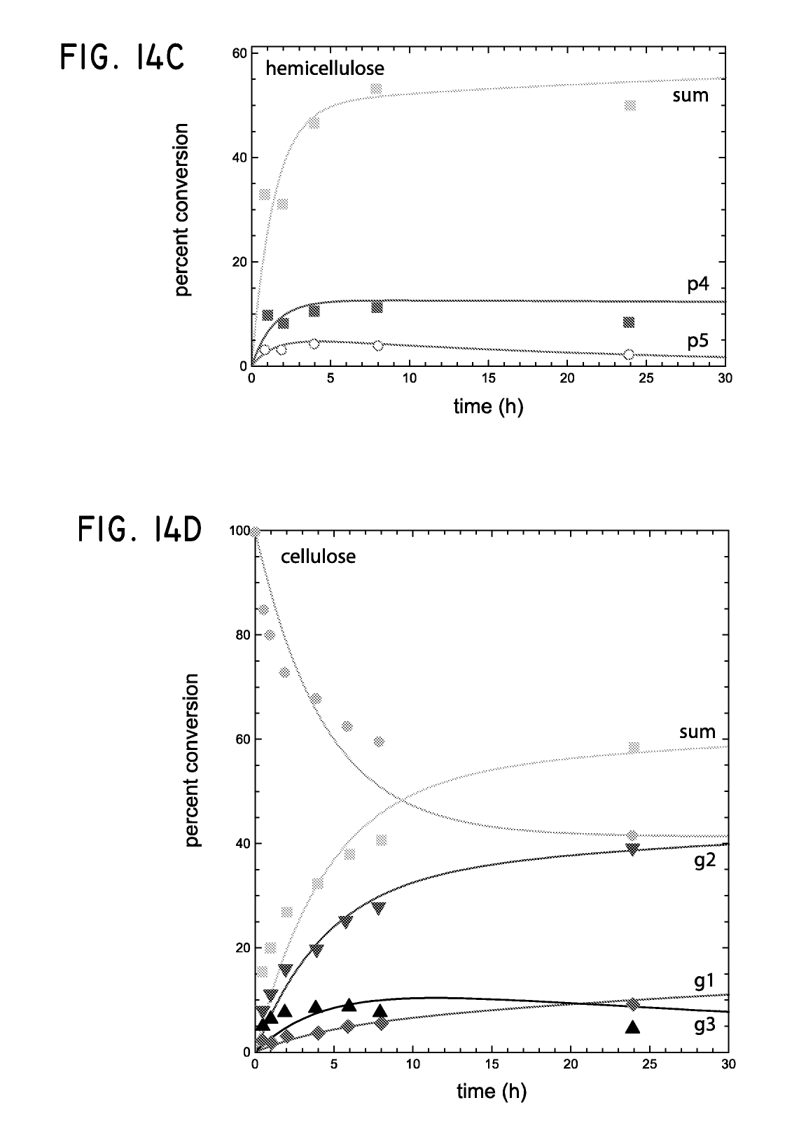

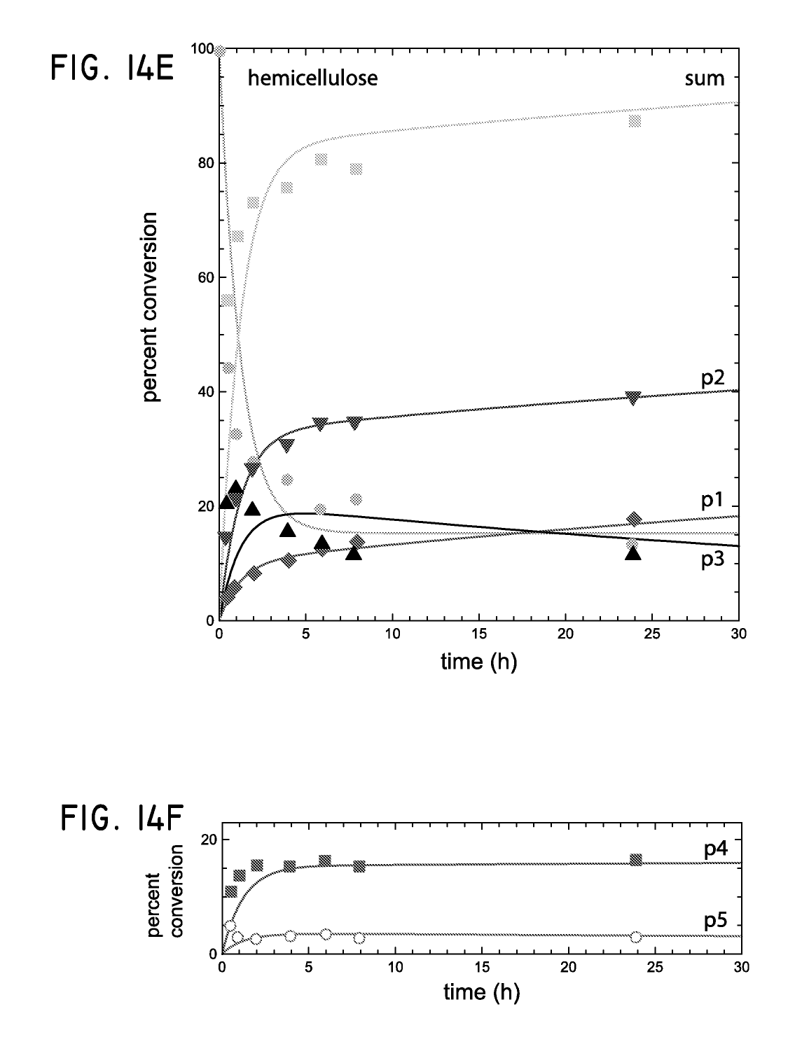

FIGS. 14A-C shows a numerical simulation of the product evolution curves for each of the individual products detected from the reaction of CelEcc_CBM3a with IL-SG. In this reaction, CelEcc_CBM3a hydrolyzed 51% of the cellulose present to a mixture of glucose (15%, relative to total cellulose in IL-SG, FIG. 14A), cellobiose (35%), and cellotriose (1%). The solid lines are solutions of the differential equations corresponding to the kinetic schemes shown in FIG. 12 (differential equations for the kinetic models are provided in the Supplementary Material). The apparent rates for each step in the kinetic models, normalized to the amount of enzyme active site present, are shown in Table 4. The simulations show that CelEcc_CBM3a has an ˜4.5-fold preference for release of cellobiose from IL-SG (k₂ versus k₁), and a small propensity to release cellotriose and to also hydrolyze solubilized cellotriose and cellobiose to smaller products. These properties correspond to reactions of purified CelEcc_CBM3a with purified cellotetraose, suggesting; that the fundamental properties of the engineered enzyme are the same in reactions with biomass and purified substrates.

I. Analysis of Hemicellulose Hydrolysis Kinetics

Simultaneous to the reaction with cellulose, CelEcc_CBM3a liberated ˜50% of the hemicellulose fraction in IL-SG (FIG. 11B). Since the NIMS analysis is based on accurate mass, it does not distinguish between the masses of various pentose isomers such as xylose, arabinose and others, generic names for pentose products are used. After 24 h of reaction, CelEcc_CBM3a gave a product distribution consisting of pentose (p1, 6%, relative to total hemicellulose in IL-SG, FIG. 14B), pentobiose (p2, 16%) and pentotriose (p3, 17%), and pentotetraose (p4, 8%, FIG. 14C) and pentopentaose (p5, 3%). Table 4 shows that the apparent rates for release of the three major products were overall similar (within ˜25%), with pentotriose accumulating with the fastest initial rate. In order to successfully model the hemicellulose product cascade, it was necessary to include slow, secondary hydrolysis of pentopentaose to pentotriose and pentobiose, and also hydrolysis of pentotetraose to 2 mol of pentobiose. Similar products were detected in reactions of purified CelEcc_CBM3a with purified xylohexaose, indicating that CelEcc_CBM3a can react with both the insoluble hemicellulose fraction as well as some of the soluble pentose oligomers released from biomass.

J. Monofunctional GH10 XynY Gave a High Yield of Pentose Products

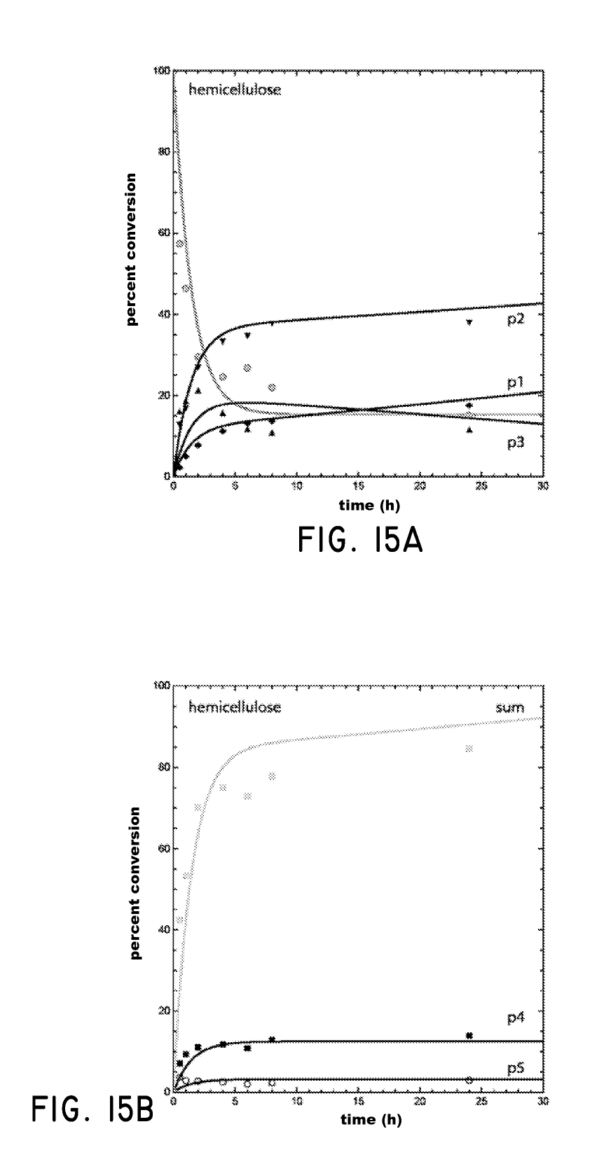

XynY is an endoxylanase comprised of CBM22, GH10 and CBM22 domains, XynY alone (8 mg enzyme/g biomass, 0.8% enzyme/biomass loading) hydrolyzed ˜85% of the xylan present in IL-SG. At 24 h of reaction, XynY gave a product distribution consisting of pentose (p1, 18%, relative to total hemicellulose in IL-SG), pentobiose (p2, 38%), pentotriose (p3, 12%), and pentotetraose (p4, 14%) and pentopentaose (p5, 3%). As with CelEcc_CBM3a, analysis of the time dependence of the product formation cascade gave numerical solutions to the kinetic scheme of FIG. 12B for hemicellulose hydrolysis (Table 4, with simulations presented in FIG. 15). The analysis revealed that pentobiose was formed at least 2× faster than pentotriose (k₂>k₃). Lesser amounts of pentose and pentotetraose were formed. Pentotriose (k₇=0.006 h⁻¹) and pentopentaose (k₇=0.013 h⁻¹) were also partially hydrolyzed later in the reaction. Although XynY reacted ˜2× faster than CelEcc_CBM3a with the hemicellulose in IL-SG, it did not react with the cellulose fraction.

K. Reactions of Combination of Enzymes.

When CelEcc_CBM3a (20 mg enzyme/g biomass) and XynY (8 mg enzyme/g biomass) were combined, an improvement in the yield of soluble sugars was obtained. FIG. 14D shows that ˜60% of the cellulose present was hydrolyzed to a mixture of glucose (9%, relative to total cellulose in IL-SG), cellobiose (45%), and cellotriose (5%), in addition, FIG. 14E shows that 86% of the hemicellulose was hydrolyzed to a mixture of pentose (17%, relative to total hemicellulose in IL-SG), pentobiose (39%), pentotriose (11%), and pentotetraose (16%) and pentopentaose (3%). Addition of a beta-glucosidase or beta-xylosidase did not further increase the yield of products, indicating that product inhibition was not likely limiting the reaction, particularly from the cellulose fraction. Moreover, when additional enzymes were added at 24 h or when the remaining biomass was washed with buffer and resuspended with new enzyme, little additional hydrolysis was observed. These results indicates that an unreactive fraction accumulated as a consequence of the progress in the hydrolysis reactions.

L. Consequences of Different Pretreatments.

The ability to perform quantitative and time-resolved analyses of products from enzymatic hydrolysis of biomass provides a unique opportunity to perform comparative studies of different chemical pretreatments. In this example, the reactivity of IL-SG was compared with an alkaline pretreatment of switchgrass (AFEX-SG) that retains nearly all chemical bond types in the original plant cell wall and thus provides a potentially more complex substrate for enzymatic hydrolysis. Table 5 shows the total products detected after normalization to the amount of enzyme active sites present in the assay reactions.

TABLE 5
Comparison of products obtained from
different pretreatments of switchgrass.

| | | hexose products | | | | | |
|---|---|---|---|---|---|---|---|
| | | (μg/nmol enzyme) | | | | | |
| Pretreatment | enzyme | g1 | g2 | g3 | sum | | |
| IL-SG | CelEcc_CBM3a | 2.15 | 1.34 | 0.03 | 3.52 | | |
| | CelB | 0.01 | 0.06 | 0.03 | 0.10 | | |
| AFEX-SG | CelEcc_CBM3a | 0.10 | 0.07 | 0.03 | 0.20 | | |
| | CelB | 0.01 | 0.01 | 0.03 | 0.05 | | |
| | | pentose products (μg/nmol enzyme) | | | | | |
| | | p1 | p2 | p3 | p4 | p5 | sum |
| IL-SG | XynY | 0.83 | 1.10 | 0.50 | 0.23 | 0.0 | 2.66 |
| | CelEcc_CBM3a | 0.28 | 0.46 | 0.28 | 0.11 | 0.0 | 1.16 |
| AFEX-SG | XynY | 0.80 | 1.10 | 0.60 | 0.30 | 0.1 | 2.85 |
| | CelEcc_CBM3a | 0.15 | 0.26 | 0.20 | 0.12 | 0.0 | 0.75 |

By comparison of the such of cellulose products, CelEcc_CBM3a was ˜35× more reactive than CelB on a molar basis toward the cellulose fraction in IL-SG (e.g., 3.52 μg total products/nmol enzyme versus 0.10 μg/mol). This relative advantage dropped to only ˜4× for reaction with the cellulose in AFEX-SG. The difference likely reflects the unchanged crystallinity of cellulose in the AFEX-SG, but perhaps also indicates blocked access to cellulose due to the increased amount of hemicellulose and lignin present. Decreases in the release of glucose and cellobiose accounted for the difference in hexose product yields from the two pretreated biomass substrates. CelEcc_CBM3a was also able catalyze hydrolysis of ˜50% of the hemicellulose present in IL-SG and ˜30% present in AFEX-SG. CelB was unable to hydrolyse the hemicellulose in either pretreated biomass. XynY gave product distributions and total product yields from the hemicellulose fraction present in AFEX-SG that were remarkably similar to the results obtained from IL-SG (Table 2). By comparison of the sum of hemicellulose products, XynY was 2× more reactive than CelEcc_CBM3a with IL-SG (2.66 μg/nmol versus 1.66 pg/nmol), and 4× more reactive with APEX-SG. These results indicate that XynY, which has both CBM22 and GH10 domains, and has been suggested to act as a processive enzyme, may be used as a pretreatment agnostic enzyme for xylan-directed hydrolysis.

As shown above, the oxime-NIMS system described herein is a rapid, specific and sensitive high-throughput mass spectrometry (MS) platform for carbohydrate analysis. Time-resolved enzyme catalysis by MS either with cellotetraose model substrate or a chemical probe for post-reaction tagging, provides insight into enzyme kinetics and interaction between biomass and enzymes. Also as described above, the oxime-NIMS assay can be used to quantitatively study reactions of recombinant cellulases produced by robotic cell-free translation with pretreated switchgrass, an emerging bioenergy crop.

### Example 4

### Versatile Synthesis of Probes for High-Throughput Enzyme Activity Screening

This example describes a synthetic route that simplifies the production of Nimzyme probes by fashioning their invariant portion as an alkylating agent. As described herein, the substrate moiety is introduced towards the end of the synthesis. Accordingly, the invariant portion of the compounds needs to be synthesized only once and a wide variety of compounds can effectively be transformed into enzyme activity probes. By fashioning this fragment as an alkylating agent, a variety of synthetically- and biologically-significant molecules can be readily transformed into Nimzyme probes. As shown herein, a chloramphenicol analog synthesized according to this methodology is effective in detecting chloramphenicol acetyltransferase (CAT) activity in crude cell lysate.

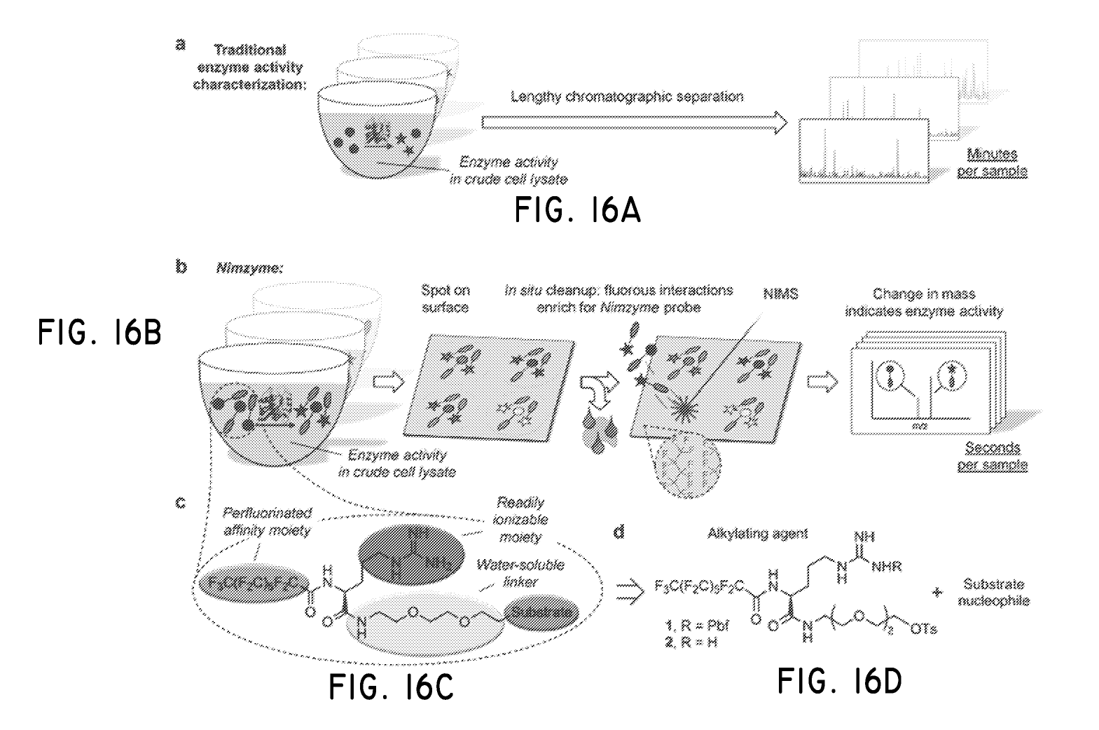

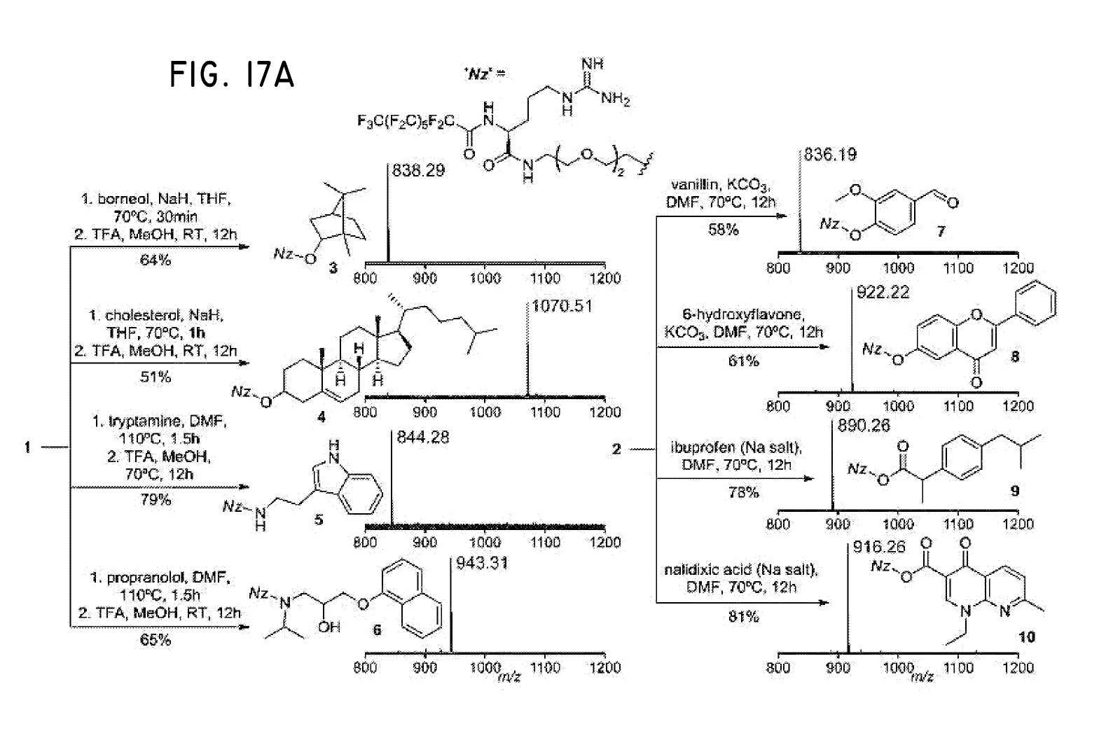

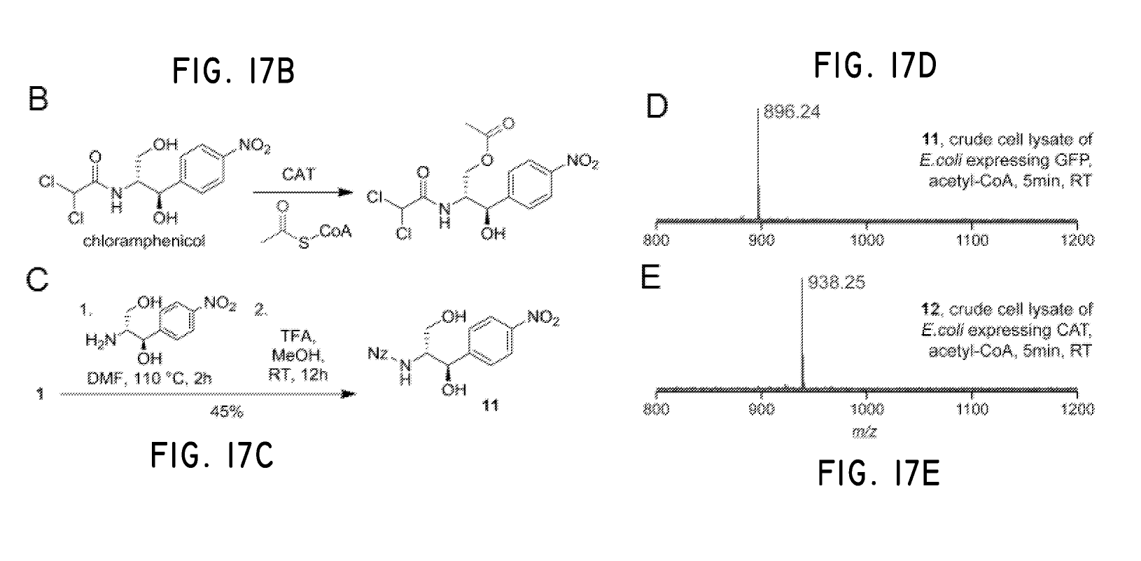

FIG. 16 shows a schematic illustration of a non-limiting example of application of the Nimzyme technology. As shown in FIG. 16A, because performing protein purification on a large number of samples is usually prohibitive, enzyme activity assays are typically performed in cell lysate, which, due to its chemical complexity, needs to be separated by chromatography before mass spectrometric analysis. FIG. 16B shows a workflow of the high-throughput Nimzyme enzyme activity assay as carried out previously. Enzyme substrate analogs harboring perfluoroalkyl moieties are incubated in a complex mixture exhibiting enzymatic activity (e.g., crude cell lysate). The mixture is quenched and spotted on a NIMS chip coated with a perfluorinated initiator substance. The surface is subjected to aqueous washes, allowing cell debris to be rinsed away while fluorous interactions with the chip surface retain the analytes. Subsequent NIMS affords an unambiguous mass spectrum, where the ratio of substrate to product mass speaks is a measure of enzyme activity.

A non-limiting generalized structure of Nimzyme-amenable substrate analogs as prepared in one example is shown in FIG. 16C. As shown in FIG. 16C, a perfluoroalkanoyl moiety imparts the required fluorophilic character to allow for on-chip purification, and an arginine moiety ensures high NIMS ionization efficiency. Substrate analogs that can be employed in the Nimzyme require a perfluorinated moiety to allow for in-situ cleanup, and an ionizable group to improve NIMS sensitivity. Avoiding reliance on the substrate moiety to impart ionizability also allows for direct comparison of NIMS peak heights to determine relative abundance. A tri(ethylene glycol) linker was installed to improve enzyme-substrate accessibility and enhance the probe's solubility in water. A p-toluenesulfonate ester (tosylate) leaving group was incorporated into the invariant portion of the Nimzyme probe, resulting in alkylating agents 1 and 2 (FIG. 16D). The invariable part of these substrates is synthesized once, in the form of tosylate alkylating agents 1 and 2. These can react with a wide variety of nucleophiles to provide. Nimzyme-amenable substrate analogs. Key: Ts=p-toluenesulfonyl; Pbf=2,2,4,6,7-pentamethyldihydrobenzofuran-5-sulfonyl.

### Synthesis of Alkylating Agents 1 and 2

A synthetic route towards alkylating agents 1 and 2 was devised, which starts from readily-available starting materials (Scheme II). 2-(2-(2-aminoethoxy)ethoxy)ethanol (13)—was obtained by means of a Gabriel synthesis from 2-(2-(2-chloroethoxy)ethoxy)ethanol—was coupled to Fmoc-Arg(Pbf)-OH to form amide 14 using conventional solution-phase peptide synthesis methodology. The Fmoc group was deprotected with diethylamine to afford amine 15, which was perfluorooctanoylated under Schotten-Baumann conditions. The resulting alcohol 16 was tosylated to afford 1 in 26% yield with respect to Fmoc-Arg(Pbf)-OH (4 steps). Lastly, the Pbf protecting group was removed with TFA/MeOH to afford 2. In scheme I, Fmoc=fluorenylmethyloxycarbonyl; Pbf=2,2,4,6,7-pentamethyldihydrobenzofuran-5-sulfonyl; Fmoc-Arg(Pbf)-OH═Nα-Fmoc-NΩ-Pbf-L-arginine; PyBOP=(benzotriazol-1-yloxy)tripyrrolidino-phosphonium hexafluorophosphate; DCM=dichloromethane; and TFA=trifluoroacetic acid.

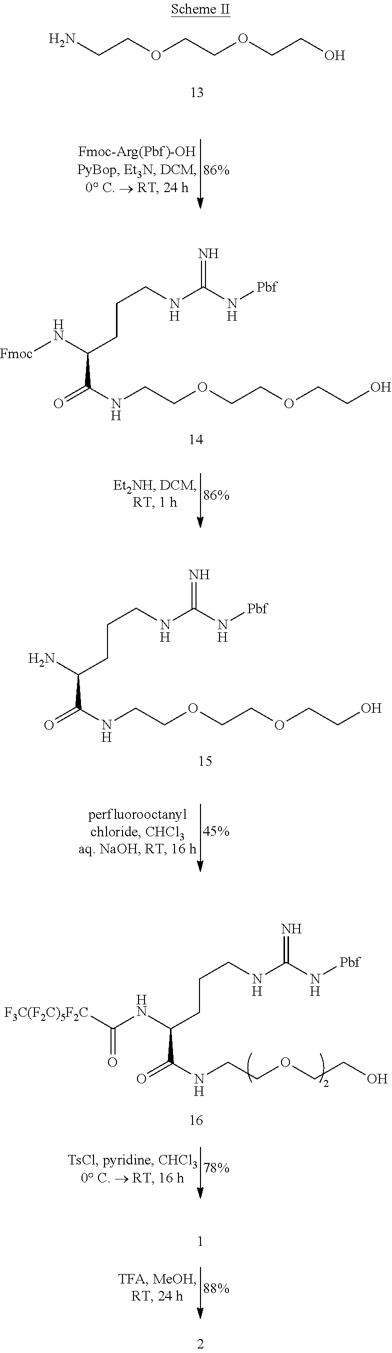

Synthetic Remarks and Materials—Moisture-free conditions were employed only where indicated. Reagents and solvents were purchased from TCI America (Cl-3G-OH), ChemPep (Fmoc-Arg(Pbf)-OH). Alfa Aesar (PFO-Cl), (EtOAc, DCM, MeOH and hexanes) and Sigma-Aldrich (all others), anhydrous whenever possible, and were used as received unless otherwise indicated. Fluoroflash fluorous solid-phase extraction cartridges were purchased from Fluorous Technologies Incorporated. Column chromatography was performed on a Teledyne Isco Combiflash Rf, using RediSep Rf Gold normal phase silica columns unless otherwise indicated. ¹H NMR and ¹³C NMR were obtained on a Bruker 600 MHz spectrometer equipped with a TCI Cryoprobe at the QB3 NMR facility. Chemical shifts are reported in ppm relative to residual solvent signal (δ¹H=7.26 and δ¹³C=77.16 for CDCl₃, and δ¹H=3.3 and δ¹³C=49.0 for CD₃OD).

Plasmid Construction—Plasmid pBbB8k-GFP was produced according to the method described in Lee et al. Journal of Biological Engineering (2011) 5:12. Construction of plasmid pBbB8k-CAT was as follows: The chloramphenicol acetyltransferase gene was PCR amplified from 10 ng pBbB8c-GFP¹ as a template and 1 μM of each primer using iProof DNA polymerase (Bio-Rad). Cycling conditions: 98° C., 2 min; 30×{98° C. 10 sec; 64° C., 10 sec; 72° C., 30 sec}; 72° C., 10 min. Forward primer contains BgIII restriction site and a RBS sequence were designed as described in Salts et al. Nature Biotechnology 2009, 27, 946-950 (predicted strength: 72268): [5′-AAAAAAAGATCT GGCTACAGCCCACTAGTAAGGAGAAGATAAATGGAGAAAAAAATCACTGGATATA C-3′ (SEQ ID NO: 1)] Reverse primer contains XhoI restriction site: [5′-AAAAAA GGATCC AAACTCGAGTTACGCCCCGCCC-3′ (SEQ ID NO:2)]. The PCR product was digested with BgIII and XhoI (Fermentas), ligated into a BlgII/XhoI digest of pBbB8k-GEP using T4 ligase (NEB), and confirmed by sequencing. The full sequence of the two plasmids mentioned in this manuscript can be downloaded from the JBEI public registry (http://public-registry.jbei.org, part IDs JPUB_{—}000129 and JPUB_{—}000580).

NIMS surface fabrication—The production of NIMS chips has been described in Woo et al. Nat. Protoc. 3:1341-1349 (2008). Briefly, a silicon wafer is cleaned thoroughly with methanol, followed by anodic etching with 25% hydrofluoric acid (w/v) in ethanol in a custom made Teflon etching chamber using a current of 2.4 A for 15 minutes. Next, chips are coated by adding the perfluorinated initiator liquid bis(heptadecafluoro-1,1,2,2-tetrahydrodecyl)tetramethyl-disiloxane for 20 minutes. Excess initiator is blown off with nitrogen.

Enzymatic activity assay—Overnight cultures of E. coli DH1 harboring either pBbB8k-GFP or pBbB8k-CAT were diluted 1:10 in LB with 50 μg mL⁻¹ kanamycin, and grown for 1 hour at 37° C., after which they were induced with 0.2% (w/v) arabinose. After 2 hours of growth, 0.5 mL of the culture was centrifuged at ˜12000×g for 1 minute, the pellet was resuspended in 0.25 mL aqueous 50 mM sodium phosphate (pH 7.5) and sonicated for 30 seconds. Acetyl-CoA was added to a final concentration of 2 mM and 11 to a final concentration of 0.5 mM. The mixture was mixed and incubated at room temperature for 5 minutes, and subsequently quenched with an equal volume of methanol. A 0.3 μL droplet of the mixture was spotted onto a NIMS chip, and the excess liquid removed 5 seconds later by touching it with a Kimwipe (Kimberly-Clark). The spotted area was washed twice with 1 μL deionized water. The NIMS chip was taped to a modified standard MALDI plate, which was then loaded into an Applied Biosystems 4800 MALDI time of flight (TOF)//TOF mass spectrometer. Aligent ESI-L Low Concentration Tuning Mix was spotted nearby on the NIMS chip to allow for mass calibration of the instrument. Spectra were acquired in manual mode and positive polarity.

Detailed Synthetic Protocols for Compounds 1-16

The alkylation and deprotection reactions were monitored by NIMS of 0.1 uL of the reaction mixture dissolved in 10 uL methanol. Fluorous Solid Phase Extraction (F-SPE) was performed on Fluoroflash 2 g cartridges as follows: The cartridges were preconditioned with 1 mL DMF and 80:20 MeOH:H₂O, loaded with the reaction mixture, washed with 7 mL MeOH:H₂O, and eluted with 10 mL MeOH. Each F-SPE cartridge was used only once. Possible product isomerism was assessed by LC/MS performed on an Agilent 1100 series HPLC. ESI source (positive polarity) and LC/MSD SL (quadrupole) set to single ion monitoring mode (chromatograms included in supplemental materials). An Inertsil ODS-3 (3 μm, 2.1×250 min) C18 column was employed. Because the quantities prepared here are too small to be accurately weighed, their yields were determined relative to a known concentration of Nz-OMe (12) by mixing them in a 1:1 ratio, and determining the ratio of NIMS peak intensities. 12 was chosen as an internal standard because its mass does not overlap with any of the reagents, products, or possible side products. It is also straightforward to prepare and can reasonably be assumed to ionize similarly to 3 through 11. It was found that, for amine nucleophiles (products 5, 6 and 11), using the hydrochloride form led to the formation of what was presumed to be ‘Nz(Pbf)-Cl’ (NIMS calc'd for [M+H]⁺: 972.2, found 972.1, characteristic 3:1 M:M+2 ratio). Hence, we either purchased or formed the free base form of these substrates. Representative reactions using alkylating agents 1 and 2 are described in detail below. All others are described below. ‘Nz’=2-(2-(Nα-perfluorooctanoylargininamidoethoxy)ethoxy)ethyl (structural formula shown in FIG. 17A)

Compound 1—Nz(Pbf)-OTs.

InChI=1S/C40H4s8F15N5O10S2/c1-21-9-11-25(12-10-21)72(65,66)69-19-18-68-17-16-67-15-14-57-30(61)27(59-31 (62)34(41,42)35(43,44)36(45,46)37(47,48)38(49,50)39(51,52)40(53,54)55)8-7-13-58-32(56)60-71(63,64)29-23(3)22(2)28-26(24(29)4)20-33(5,6)70-28/h9-12,27H,7-8,13-20H2,1-6H3,(H,57,61)(H,59,62)(H3,56,58,60)/t27−/m0/s1

A solution of 359 mg (0.38 mmol) 6, 273 mg (1.4 mmol) tosyl chloride and 0.122 μL (1.9 mmol) pyridine in 20 mL chloroform was stirred at room temperature for 16 h. The mixture was washed with 100 mL 1M aqueous HCl and extracted three times with 50 mL chloroform. The extract was concentrated in vacuo and purified by flash chromatography (9:1 EtOAc:Hexane) to afford the title compound as a white solid (326 mg, 0.29 mmol, 78% yield).

¹H NMR (600 MHz, CDCl₃) δ 7.76 (d, J=7.9 Hz, 2H), 7.63 (d, J=7.8 Hz, 1H), 7.39 (bs, 1H), 7.34 (d, J=7.9 Hz, 2H), 4,69 (td, J=8.5, 4.9 Hz, 1H), 4.14 (t, J=4.5 Hz, 2H). 3.73-3.07 (m, 12H), 2.94 (s, 2H), 2.58 (s, 3H). 2.51 (s, 3H), 2.44 (s, 3H), 2.08 (s, 3H), 1.99-1.48 (m, 6H). 1.45 (s, 6H). ¹³C NMR (151 MHz, CDCl₃) δ 170.51, 158.99, 157.54 (t, ³J_{C—F}=26.2 Hz), 156.54, 145.40, 138.51, 132.88, 132.66, 132.46, 130.15, 128.01, 124.86, 117.72, 118.78-107.92 (m), 86.58, 77.37, 77.16, 76.95, 70.79, 70.14, 69.79, 69.38, 68.70, 52.88, 43.36, 39.59, 30.47, 28.70, 25.36, 21.75, 19.39, 18.06, 12.56. NIMS calc'd for [M+H]⁺: 1108.27, found: 1108.26

Compound 2—Nz-OTs.

InChI=1S/C27H32F15N5O7S/c1-15-4-6-16(7-5-15)55(50,51)54-14-13-53-12-11-52-10-9-45-18(48)17(3-2-8-46-20(43)44)47-19(49)21(28,29)22(30,31)23(32,33)24(34,35)25(36,37)26(38,39)27(40,41)42/h4-7,17H,2-3,8-14H2,1H3(H,45,48)(H,47,49)(H4,43,44,46)/t17-/m0/s1

27.8 mg (25.1 μmol) 1 was stirred in 90:10 TFA:MeOH at room temperature for 12 h, after which the TFA:MeOH was evaporated. The residue was washed three times with toluene, three times with hexanes, redissolved in MeOH, and the MeOH evaporated in vacuo. These washing steps were repeated two more times, affording the title compound as a colorless residue (21.4 mg, 22.0 μmol, 88% yield).

¹H NMR (600 MHz, MeOD) δ 7.79 (d, J=8.3 Hz, 2H), 7.44 (d, J=7.9 Hz, 2H), 4.47 (dd, J=8.8, 5.9 Hz, 1H), 4.16 (dd, J=5.3, 3.6 Hz, 2H), 3.69 (s, 1H), 3.67 (dd, J=5.2, 2,8 Hz, 2H), 3.58-3.50 (m, 6H), 3.43-3.34 (m, 2H), 3.21 (t, J=7.0 Hz, 2H), 2.45 (s, 3H), 1.94 (ddt, J=13.6, 10,2, 5.9 Hz, 1H), 1.85-1.77 (m, 1H), 1.71-1.56 (m, 2H). ¹³C NMR (151 MHz, MeOD) δ 172.37, 157.204 (t, ³J_{C—F}=26.2 Hz), 158.66, 146.56, 134.43, 131.07, 129.03, 119.00-106.00 (m), 71.58, 71.18, 70.93, 70.38, 69.78, 55.13, 49.00, 41.82, 40.50, 29.80, 26.26, 21.55. NIMS calc'd for [M+H]⁺: 856.19, found 856.18.

The side product washed away in the hydrophobic washes was determined to be methyl 2,2,4,6,7-pentamethyl-2,3-dihydrobenzofuran-5-sulfonate (MeOPbf) by NMR.

Compound 3-O-Nz-Borneol

InChI=1S/C30H42F15N5O5/c1-22(2)16-6-7-23(22,3)18(15-16)55-14-13-54-12-11-53-10-9-48-19(51)17(5-4-8-49-21(46)47)50-20(52)24(31,32)25(33,34)26(35,36)27(37,38)28(39,40)29(41,42)30(43,44)45/h16-18H,4-15H2,1-3H3,(H,48,51)(H,50,52)(H4,46,47,49)/t16?,17−,18?,23?/m0/s1

Into a borosilicate test tube with stir bar was added 250 μL 1M borneol (50 eq) in THF and 15 mg 60% (w/w) NaH (75 eq), which was left to stir for 5 min at RT. Then, 50 μL 100 mM Nz(Pbf)-OTs (1) in DMF was added, and the mixture was stirred at RT for 2 h. The reaction was quenched with 250 μL saturated ammonium chloride, and the mixture was purified by F-SPE. The eluent was evaporated in vacuo, redissolved in 2 mL 90:10 TFA:MeOH and left to stir at RT for 12 h. The TFA:MeOH was evaporated under a gentle stream of nitrogen, leaving a colorless residue which was taken up in MeOH. 64% yield. NIMS calc'd for [M+M]⁺: 838.30, found 838.29.

Also found was 35% of what is presumed to be the E2 elimination product (NIMS calc'd for [M+H]⁺: 684.16, found 684.14).

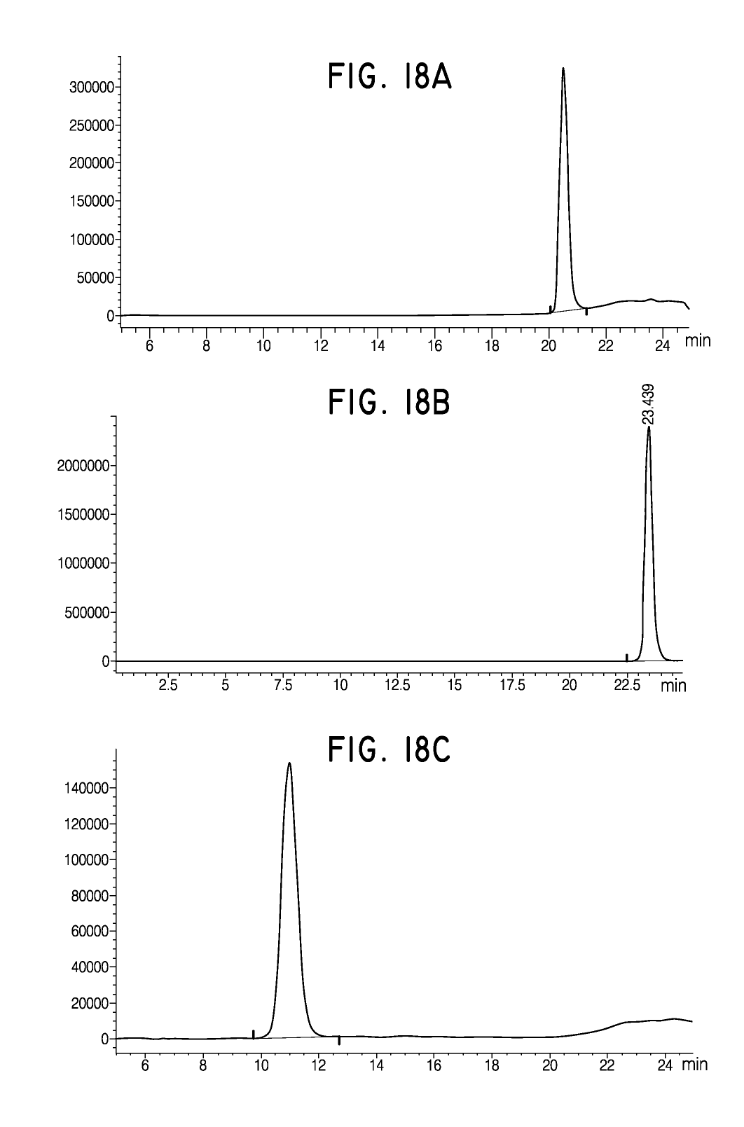

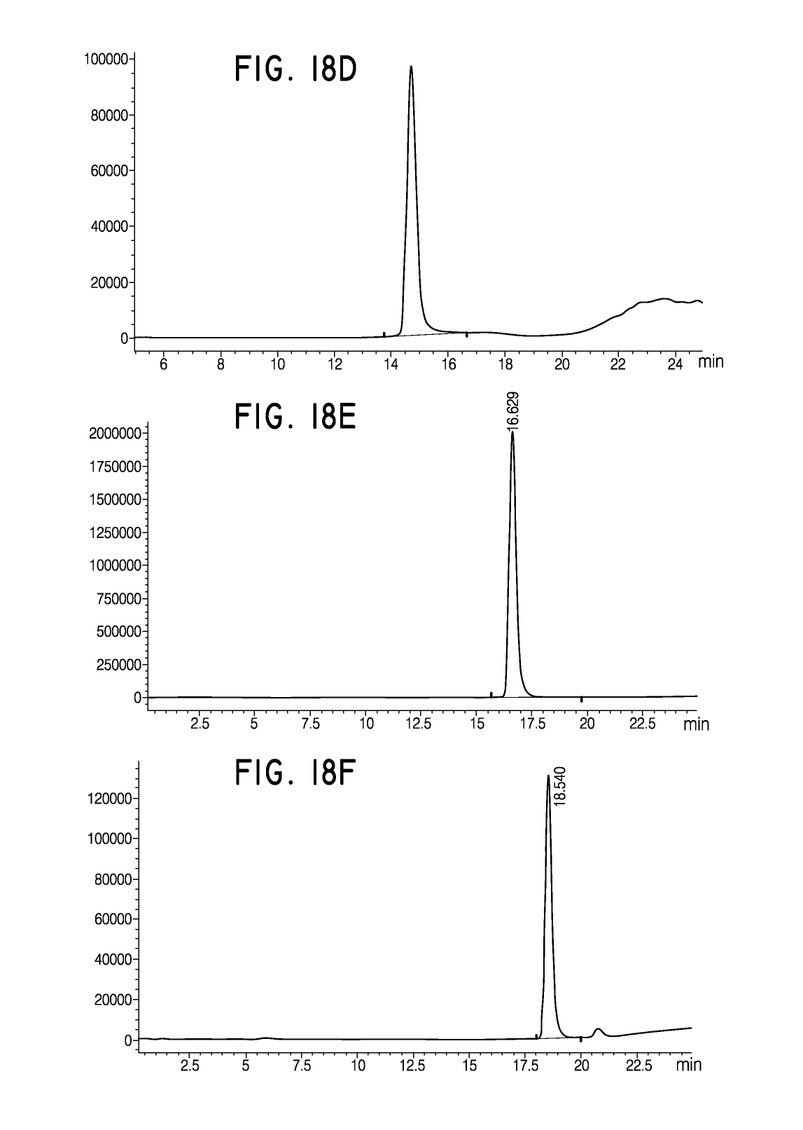

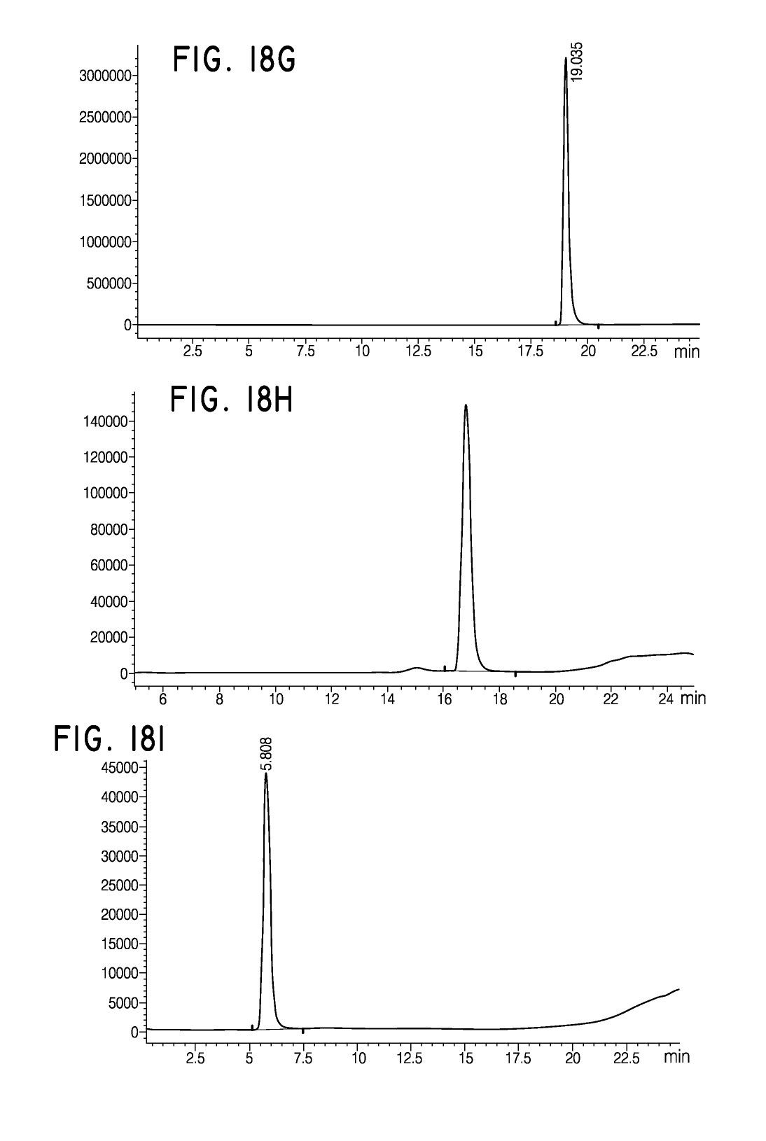

FIG. 18A is a LC/MS chromatogram showing the purity of 3 (m/z=838.3).

Compound 4—O-Nz-cholesterol.

InChI=1S/C47H70F15N5O5/c1-27(2)8-6-9-28(3)32-13-14-33-31-12-11-29-26-30(15-17-39(29,4)34(31)16-18-40(32,33)5)72-25-24-71-23-22-70-21-20-65- 36(68)35(10-7-19-66-38(63)64)67-37(69)41(48,49)42(50,51)43(52,53)44(54,55)45(56,57)46(58,59)47(60,61)62/h11,27-28,30-35H,6-10,12-26H2,1-5H3,(H,65,68)(H,67,69)(H4,63,64,66)/t28−,30?,31+,32−,33+,34+,35+,39+,40−/m1/s1

Into a borosilicate test tube with stir bar was added 250 μL 1M cholesterol (50 eq) in THF and 10 mg 60% (w/w) NaH (50 eq), which was left to stir for 5 min at RT. Then, 50 μL 100 mM Nz(Pbf)-OTs (1) in DMF was added, and the mixture was stirred at RT for 2 h. The reaction was quenched with 250 μL saturated ammonium chloride, passed through a cotton plug and purified by F-SPE. The eluent was evaporated in vacuo, redissolved in 2 mL 90:10 TFA:MeOH and left to stir at RT for 15 min (longer deprotection times show ether cleavage, probably due to its allylic nature). The TFA:MeOH was evaporated under a gentle stream of nitrogen, leaving a white residue which was taken up in MeOH.

51% yield. NIMS calc'd for [M+H]⁺: 1070.52, found 1070.51.

Also found was 10% elimination product (see 3—O-Nz borneol). FIG. 18B is a LC/MS chromatogram showing the purity of 4 (m/z=1070.5).

Compound 5—N-Nz-tryptamine.

InChI=1S/C30H36F15N7O4/c31-24(32,25(33,34)26(35,36)27(37,38)28(39,40)29(41,42)30(43,44)45)22(54)52-20(6-3-8-50-23(46)47)21(3)49-11-13-56-15-14-55-12-10-48-9-7-17-16-51-19-5-2-1-4-18(17)19/h1-2,4-5,16,20,48,51H,3,6-15H2,(H,49,53)(H,52,54)(H4,46,47,50)/t20−/m0/s1

Into a borosilicate test tube with stir bar was added 50 μL 100 mM Nz(Pbf)-OTs (1) in DMF, 200 μL more DMF, and 40.1 mg (250 μmol, 50 eq) tryptamine. The mixture was stirred at 110° C. for 2 h, cooled to RT and F-SPE purified. The eluent was evaporated in vacuo, redissolved in 2 ml, 90:10 TFA:MeOH and stirred at 70° C. for 12 h. The TFA:MeOH was evaporated under a gentle stream of nitrogen, leaving a colorless residue which was taken up in MeOH.

79% yield. NIMS calc'd for [M+H]⁺: 844.27, found 844.28 FIG. 18C is a LC/MS chromatogram showing the purity of 5 (m/z=844.3).

Compound 6—N-Nz-propranolol.

InChI=1S/C36H45F15N6O6/c1-21(2)57(19-23(58)20-63-26-11-5-8-22-7-3-4-9-24(22)26)14-16-62-18-17-61-15-13-54-27(59)25(10-6-12-55-29(52)53)56-28(60)30(37,38)31(39,40)32(41,42)33(43,44)34(45,46)35(47,48)36(49,50)51/h3-5,7-9,11,21,23,25,58H,6,10,12-20H2,1-2H3,(H,54,59)(H,56,60)(H4,52,53,55)/t23?,25−/m0/s1

100 mg propranolol HCl was mixed with 1 mL 0.5 M NaOH and extracted with 3×1 mL EtOAc, which was evaporated in vacuo to yield propranolol base as a white powder. 64.8 mg (250 μmol, 50 eq) of this was added into a borosilicate test tube with stir bar, 200 μL DMF and 50 μL 100 mM Nz(Pbf)-OTs (1) in DMF. The mixture was stirred at 110° C. for 2 h, cooled to RT and F-SPE purified. The eluent was evaporated in vacuo, redissolved in 2 mL 90:10 TFA:MeOH and stirred at RT for 12 h. The TFA:MeOH was evaporated under a gentle stream of nitrogen, leaving a colorless residue which was taken up in MeOH.

65% yield. NIMS calc'd for [M+H]⁺: 943.32, found 943.31. FIG. 18D is a LC/MS chromatogram showing the purity of 6 (m/z=943.3).

Compound 7—O-Nz-vanillin ether.

InChI=1S/C28H32F15N5O7/c1-52-18-13-15(14-49)4-5-17(18)55-12-11-54-10-9-53-8-7-46-19(50)16(3-2-6-47-21(44)45)48-20(51)22(29,30)23(31,32)24(33,34)25(35,36)26(37,38)27(39,40)28(41,42)43/h4-5,13-14,16H,2-3,6-12H2,1H3,(H,46,50)(H,48,51)(H4,44,45,47)/t16−/m0/s1

Into a borosilicate test tube with stir bar was added 3.8 mg vanillin (2.5 μmol, 50 eq), 490 μL DMF and 6.9 mg (50 μmol, 100 eq) KCO₃. The mixture was stirred at 70° C. for 1 minute, 10 μL 50 mM Nz-OTs (2) was added, and the mixture was stirred for at 70° C. for 3 h. After cooling to RT, 5 mL 1M aqueous NaOH was added, and the resulting solution was extracted with 3×5 mL chloroform, each of the extracts in turn washed with 5 mL 1 M aqueous NaOH. A few crystals of NH₃Cl were added to the chloroform extracts, the solution filtered and evaporated in vacuo to yield a colorless residue.

58% yield. NIMS calc'd for [M+H]⁺: 836.21, found 836.19. FIG. 18E is a LC/MS chromatogram showing the purity of 7 (m/z=836.2).

Compound 8—6-Nz-flavone ether.

InChI=1S/C35H34F15N5O7/c36-29(37,30(38,39)31(40,41)32(42,43)33(44,45)34(46,47)35(48,49)50)27(58)55-22(7-4-10-54-28(51)52)26(57)53-11-12-59-13-14-60-15-16-61-20-8-9-24-21(17-20)23(56)18-25(62-24)19-5-2-1-3-6-19/h1-3,5-6,8- 9,17-18,22H,4,7,10-16H2,(H,53,57)(H,55,58)(H4,51,52,54)/t22−/m0/s1

Into a borosilicate test tube with stir bar was added 6.0 mg vanillin (25 μmol, 50 eq), 490 μL DMF and 6.9 mg (50 μmol, 100 eq) KCO₃. The mixture was stirred at 70° C. for 1 minute, 10 μL, 50 mM Nz-OTs (2) was added, and the mixture was stirred for at 70° C. for 3 h. After cooling to RT, 5 mL 1M aqueous NaOH was added, and the resulting solution was extracted with 3×5 mL chloroform, each of the extracts in turn washed with 5 mL 1 M aqueous NaOH. A few crystals of NH₃Cl were added to the chloroform extracts, the solution filtered and evaporated in vacuo to yield a colorless residue.

61% yield. NIMS cal'd for [M+H]⁺: 922.23, found 922.22. FIG. 18F is a LC/MS chromatogram showing the purity of 8 (m/z=922.2).

Compound 9—Ibuprofen Nz ester.

InChI=1S/C33H42F15N5O6/c1-18(2)17-20-6-8-21(9-7-20)19(3)24(55)59-16-15-58-14-13-57-12-11-51-23(54)22(5-4-10-52-26(49)50)53-25(56)27(34,35)28(36,37)29(38,39)30(40,41)31(42,43)32(44,45)33(46,47)48/h6-9,18-19,22H,4-5,10-17H2,1-3H3,(H,51,54)(H,53,56)(H4,49,50,52)/t19?,22−/m0/s1

Into a borosilicate test tube with stir bar was added 5.7 mg ibuprofen sodium salt (25 μmol, 50 eq), 10 μL. 50 mM Nz-OTs (2) and 490 uL more DMF was added, and the mixture was stirred for at 70° C. for 3 h. After cooling to RT, 5 mL 1M aqueous NaOH was added, the resulting solution was extracted with 3×5 mL chloroform, and each of the extracts was in turn washed with another 5 mL 1M aqueous NaOH. A few crystals of NH₃Cl were added to the chloroform extracts, which were filtered and evaporated in vacuo to yield a colorless residue which was taken up in MeOH 78% yield. NIMS calc'd for [M+H]⁺: 890.30, found 890.26

FIG. 18G is a LC/MS chromatogram showing the purity of 9 (m/z=890.3).

Compound 10—Naladixic acid Nz ester.

InChI=1S/C32H36F15N7O7/c1-3-54-15-18(20(55)17-7-6-16(2)52-21(17)54)23(57)61-4-13-60-12-11-59-10-9-50-22(56)19(5-4-8-51-25(48)49)53- 24(58)26(33,34)27(35,36)28(37,38)29(39,40)30(41,42)31(43,44)32(45,46)47/h6-7,15-19H,3-5,8-14H2,1-2H3,(H,50,56)(H,53,58)(H4,48,49,51)/t19−/m0/s1

Into a borosilicate test tube with stir bar was added 6.4 mg nalidixic acid sodium salt (25 μmol, 50 eq), 10 μL 50 mM Nz-OTs (2) and 490 μL more DMF was added, and the mixture was stirred for at 70° C. for 3 h. After cooling to RT, 5 mL 1 M aqueous NaOH was added, the resulting solution was extracted with 3×5 mL chloroform, and each of the extracts was in turn washed with 5 mL 1 M aqueous NaOH. A few crystals of NH₃Cl were added to the chloroform extracts, which were filtered and evaporated in cacao to yield a colorless residue.

81% yield NIMS calc'd for [M+H]⁺: 916.25, found 916.26. FIG. 18H is a LC/MS chromatogram showing the purity of 10 (m/z=916.3).

Compound 11—(1R,2R)-N-Nz-1-(4-nitrophenyl)propane-1,3-diol.

InChI=1S/C29H36F15N7O8/c30-23(31,24(32,33)25(34,35)26(36,37)27(38,39)28(40,41)29(42,43)44)21(55)50-17(2-1-7-49-22(45)46)20(54)48-9-11-59-13-12-58-10-8-47-18(14-52)19(53)15-3-5-16(6-4-15)51(56)57/h3-6,17-19,47,52-53H,1-2,7-14H2,(H,48,54)(H,50,55)(H4,45,46,49)/t17−,18+,19+/m0/s1

Into a borosilicate test tube with stir bar was added 50 μL 100 mM Nz(Pbf)-OTs (1) in DMF, 200 μL more DMF, and 53.1 mg (250 umol, 50 eq) (1R,2R)-2-Amino-1-(4-nitrophenyl)-1,3-propanediol. The mixture was stirred at 110° C. for 2 h, cooled to RT and F-SPE purified. The eluent was evaporated in vacuo, redissolved in 2 mL 90:10 TFA:MeOH and left to stir at RT for 12 h. The TFA:MeOH was evaporated under a gentle stream of nitrogen, leaving a white residue which was taken up in MeOH 45% yield. NIMS calc'd for [M+H]⁺: 896.25, found 896.24. FIG. 18I is a LC/MS chromatogram showing the purity of 11 (m/z=896.3).

Compound 12—Nz-OMe.

InChI=1S/C21H28F15N5O5/c1-44-7-8-46-10-9-45-6-5-39-12(42)11(3-2-4-40-14(37)38)41-13(43)15(22,23)16(24,25)17(26,27)18(28,29)19(30,31)20(32,33)21(34,35)36/h11H,2-10H2,1H3,(H,39,42)(H,41,43)(H4,37,38,40)/t11−/m0/s1

Into a borosilicate test tube with stir bar was added 50 μL 100 mM Nz(Pbf)-OTs (1) in DMF. 100 μL more DMF and 50 μL 25% (w/v) sodium methoxide, and the mixture was left to stir vigorously at RT for 1 h. 2 mL saturated aqueous NH₄Cl was added, and the mixture was extracted with 3×5 mL EtOAc. The EtOAc extract was evaporated in vacuo, redissolved in 2 mL 90:10 TFA:MeOH and left to stir at RT for 12 h. The TFA:MeOH was evaporated under a gentle stream of nitrogen, leaving a colorless residue. The yield was assumed to be 5 μmol. No loss in yield (as measured by NIMS peak intensity ratios versus Nz-OTs) was observed when the EtOAc extract was washed with water (hence, partitioning of the intermediate into water rather than EtOAc is negligible), and no loss in yield was observed when the TFA deprotection steps were carried out for an extended time (up to 48 hours).

NIMS calc'd for [M+H]⁺: 716.192, found 716.195

Compound 13—2-(2-(2-aminoethoxy)ethoxy)ethanol.

InChI=1S/C6H15NO3/c7-1-3-9-5-6-10-4-2-8/h8H,1-7H2

A solution of 53.0 g (0.31 mol) 2-(2-(2-chloroethoxy)ethoxy)ethanol and potassium phthalimide (0.33 mol) in 200 mL DMF was stirred at 130° C. for 12 h. The yellow solution was concentrated in vacuo, resuspended in 200 mL deionized water, extracted three times with 200 mL ethyl acetate. The extract was concentrated in vacuo and resuspended in 1.5 L 95% ethanol. 30 mL hydrazine monohydrate was added, and the mixture was stirred at 130° C. Clumps that formed during the course of the reaction were carefully broken up using a spatula. After 12 h, 30 mL concentrated hydrochloric acid was added, and the mixture was stirred at 130° C. for another 2 h. After letting the mixture cool to room temperature, it was concentrated in vacuo and resuspended in 1 L deionized water. The suspension was filtered and to the filtrate was added 15 mL 10N NaOH. The mixture was evaporated in vacuo and the residue was extracted with 500 mL DCM, which was concentrated in vacuo to afford the title compound as a yellow oil without need for further purification (38.04 g, 0.23 mol, 73% yield).

¹H NMR (600 MHz, CDCl₃) δ 3.59 (t, J=4.8 Hz, 2H), 3.56-3.54 (m, 2H), 3.53-3.51 (m, 2H), 3.48 (t, J=4.5 Hz, 2H), 3.42 (t, J=5.2 Hz, 2H), 2.74 (t, J=5.3 Hz, 2H), 2.51 (s, 1H). ¹³C NMR (151 MHz, CDCl₃) δ 72.96, 72.78, 70.29, 70.14, 61.14, 41.33. ESI quadrupole MS calc'd for [M+H]⁺: 150.1, found 150.1

Compound 14—Fmoc-Arg(Pbf)-NH-(Et-O)₂-Et-OH.

InChI=1S/C40H53N5O9S/c1-25-26(2)36(27(3)32-23-40(4,5)54-35(25)32)55(49,50)45-38(41)43-16-10-15-34(37(47)42-17-19-51-21-22-52-20-18-46)44-39(48)53-24-33-30-13-8-6-11-28(30)29-12-7-9-14-31(29)33/h6-9,11-14,33-34,46H,10,15-24H2,1-5H3,(H,42,47)(H,44,48)(H3,41,43,45)/t34−/m0/s1.

To 20 mL DCM stirring in a flame-dried round-bottom flask at 0° C., was added 492 mg (3.3 mmol) 3, 1.96 g Fmoc-Arg(Pbf)-OH (3 mmol), 219 μL triethylamine (3.3 mmol) and 1.72 g PyBOP (3.3 mmol). The reaction was allowed to warm up to room temperature while stirring for 24 h. 50 mL 5% Aqueous NH₃Cl was added and extracted with 3×50 mL DCM. The combined extracts were concentrated in vacuo (addition of a few mL toluene reduces foaming). The crude product was purified by flash chromatography (19:1 DCM:MeOH) and dried in vacuo to afford the title compound as a white solid (2.00 g, 2.57 mmol, 86% yield).

¹H NMR (600 MHz, CDCl3) δ 7.72 (d, J=7.6 Hz, 2H), 7.56 (d, J=10.5 Hz, 2H), 7.35 (t, J=7.2 Hz, 2H), 7.25 (t, J=7.6 Hz, 2H), 6.36 (s, 1H), 6.29 (s, 1H), 4.41-4.28 (m, 2H), 4.27-4.19 (m, 1H), 4.18-4.09 (m, 2H), 3.90 (s, 3.75-3.63 (m, 2H), 3.63-3.47 (m, 6H), 3.48-3.39 (m, 2H), 3.30-3.16 (m, 2H), 2.90 (s, 2H), 2.58 (s, 3H), 2.50 (s, 3H), 2.42 (s, 1H), 2.06 (s, 3H). 1.94-1.80 (m, 2H), 1.75-1.63 (m, 2H), 1.63-1.50 (m, 2H), 1.42 (s, 6H), ¹³C NMR (151 MHz, CDCl3) δ 171.16, 158.75, 156.54, 156.47, 143.85, 143.75, 141.26, 138.32, 132.89, 132.25, 127.70, 127.09, 125.13, 124.64, 119.94, 117.53, 86.38, 72.43, 70.14, 70.01, 69.36, 66.96, 61.40, 60.40, 53.43, 49.16, 47.14, 43.23, 39.27, 33.89, 30.24, 28.57, 25.61, 25.31, 24.96, 21.05, 19.30, 17.97, 14.21, 12.47. NIMS calc'd for [M+H]⁺: 780.36, found 780.35.

Compound 15—H2N-Arg(Pbf)-NH-(Et-O)₂-Et-OH.

InChI=1S/C25H43N5O7S/c1-16-17(2)22(18(3)19-15-25(4,5)37-21(16)19)38(33,34)30-24(27)29-8-6-7-20(26)23(32)28-9-11-35-13-14-36-12-10-31/h20,31H,6-15,26H2,1-5H3,(H,28,32)(H3,27,29,30)/t20−/m0/s1

1.56 g (2.0 mmol) 4 was dissolved in 100 mL 1:1 DCM:diethylamine, and stirred at room temperature for 1 h. 50 mL toluene was added and the mixture was concentrated under reduced pressure. The crude product was purified by flash chromatography (9:1 DCM:MeOH) and dried in vacuo to afford the title compound as a hygroscopic white solid (0.96 g, 1.72 mmol, 86% yield).

¹H NMR (600 MHz, CDCl₃) δ 8.01 (s, 1H), 6.57 (s, 1H), 4.68 (s, 2H), 3.71-3.63 (m, 3H), 3.61-3.49 (m, 6H), 3.46-3.36 (m, 2H), 3.23-3.17 (m, 2H), 2.93 (s, 2H), 2.55 (s, 3H), 2.47 (s, 3H), 2.06 (s, 3H), 1.94-1.88 (m, 0H), 1.86-1.76 (m, 2H), 1.74-1.64 (m, 2H), 1.64-1.55 (m, 4H), 1.43 (s, 6H). ¹³C NMR (151 MHz, CDCl3) δ 173.51, 158.78, 156.76, 138.29, 133.05, 132.25, 124.71, 117.58, 86.48, 77.16, 72.54, 70.17, 70.10, 69.59, 61.25, 53.96, 43.36, 39.28, 31.03, 28.70, 25.20, 19,38, 18.06, 12.57. NIMS calc'd for [M+H]⁺: 558.296, found 558.303.

Compound 16—Nz(Pbf)-OH.

InChI=1S/C33H42F15N5O8S/c1-16-17(2)22(18(3)19-15-26(4,5)61-21(16)19)62(57,58)53-25(49)51-8-6-7-20(23(55)50-9-11-59-13-14-60-12-10-54)52-24(56)27(34,35)28(36,37)29(38,39)30(40,41)31(42,43)32(44,45)33(46,47)48/h20,54H,6-15H2,1-5H3,(H,50,55)(H,52,56)(H3,49,51,53)/t20−/m0/s1

To a stirring solution of 1.06 g (1.9 mmol) 5 in a mixture of 50 mL chloroform and 10 mL 10N NaOH at 0° C., 473 μL (1.9 mmol) perfluorooctanoyl chloride was added, after which the reaction was allowed to warm up to room temperature, while stirring, for 16 h. 100 mL water was added, the mixture was extracted with 3×100 mL chloroform, and the combined extracts were concentrated in vacuo (addition of a few mL methanol reduces foaming). The crude product was purified by flash chromatography (19:1 DCM:MeOH) and dried in vacuo to afford the title compound as a white solid (820 mg, 0.86 mmol, 45% yield).

¹H NMR (600 MHz, CDCl₃) δ 7.92 (d, J=7.7 Hz, 1H), 7.62 (t, J=5.7 Hz, 1H), 6.37 (s, 3H), 4.62 (td, J=8.3, 5.1 Hz, 1H), 3.84 (s, 1H). 3.72 (s, 2H), 3.62-3.13 (m, 12H), 2.94 (s, 2H), 2.56 (s, 3H), 2.48 (s, 3H), 2.08 (s, 3H), 2.00-1.50 (m, 4H), 1.45 (s, 6H). ¹³C NMR (151 MHz, CDCl₃) δ 170.66, 159.02, 157.72 (t, ³J_{C—F}=26.2 Hz), 156.67, 138.44, 132.75, 132.37, 124.87, 117.76, 118.52-106.88 (m), 86.60, 77.16, 72,48, 70.26, 70.11, 69.37, 61.55, 53.24, 43.34, 39.52, 30.08, 28.67, 25.27, 19.32, 18.01, 12.50. NIMS calc'd for 954.26, found 954.28.

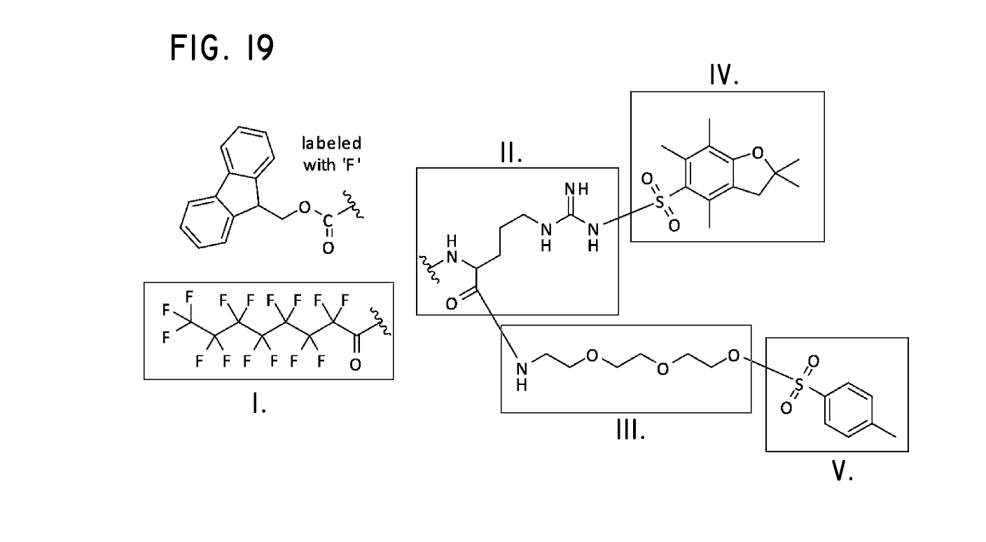

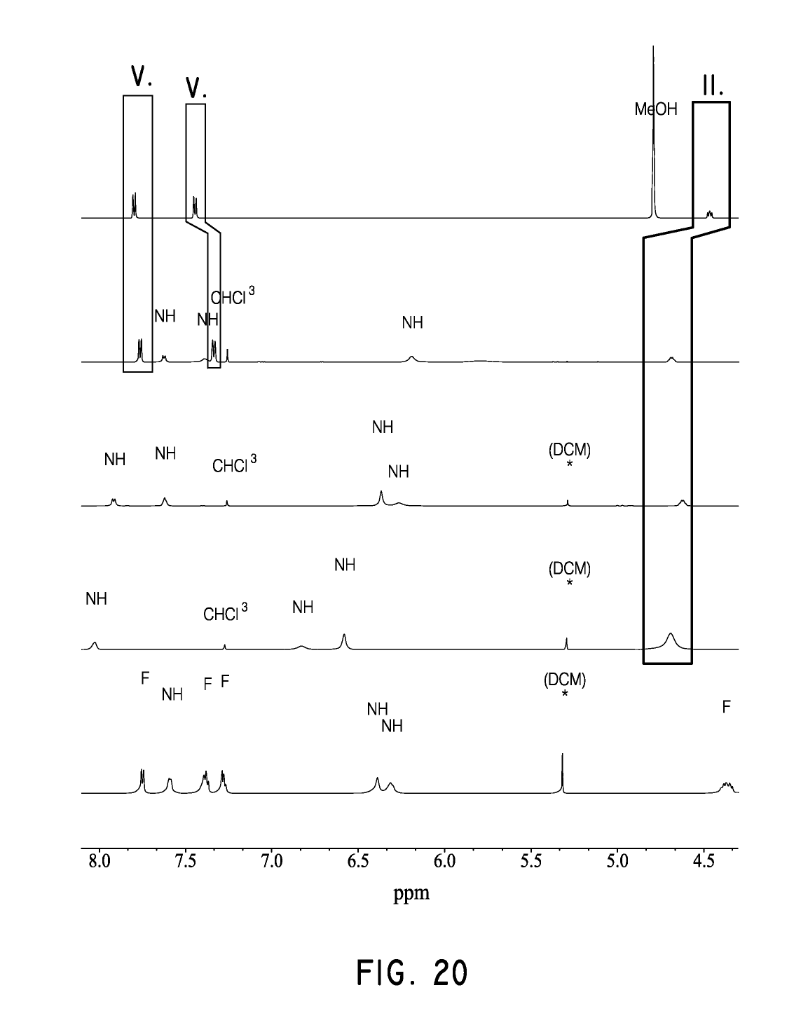

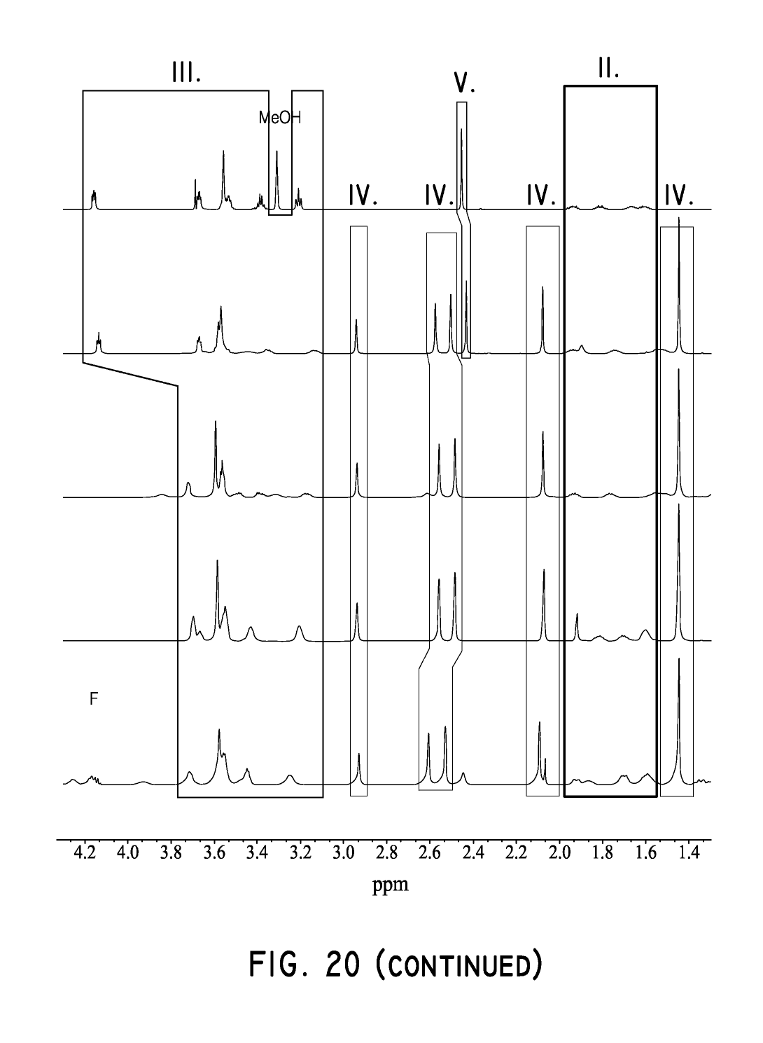

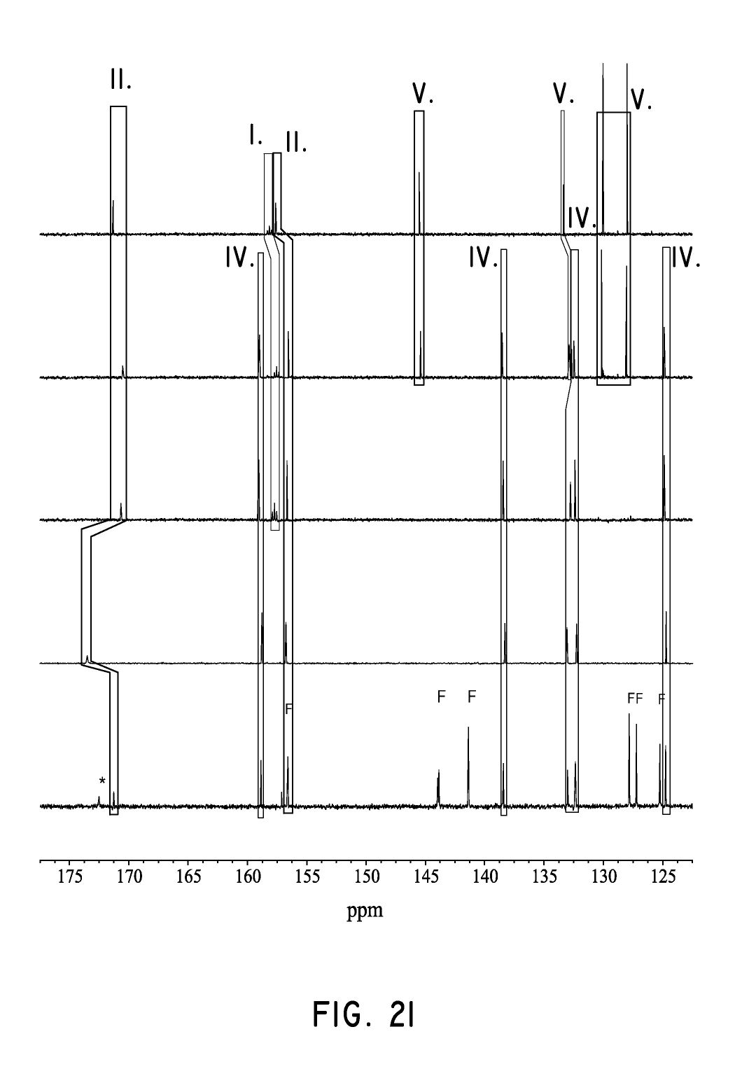

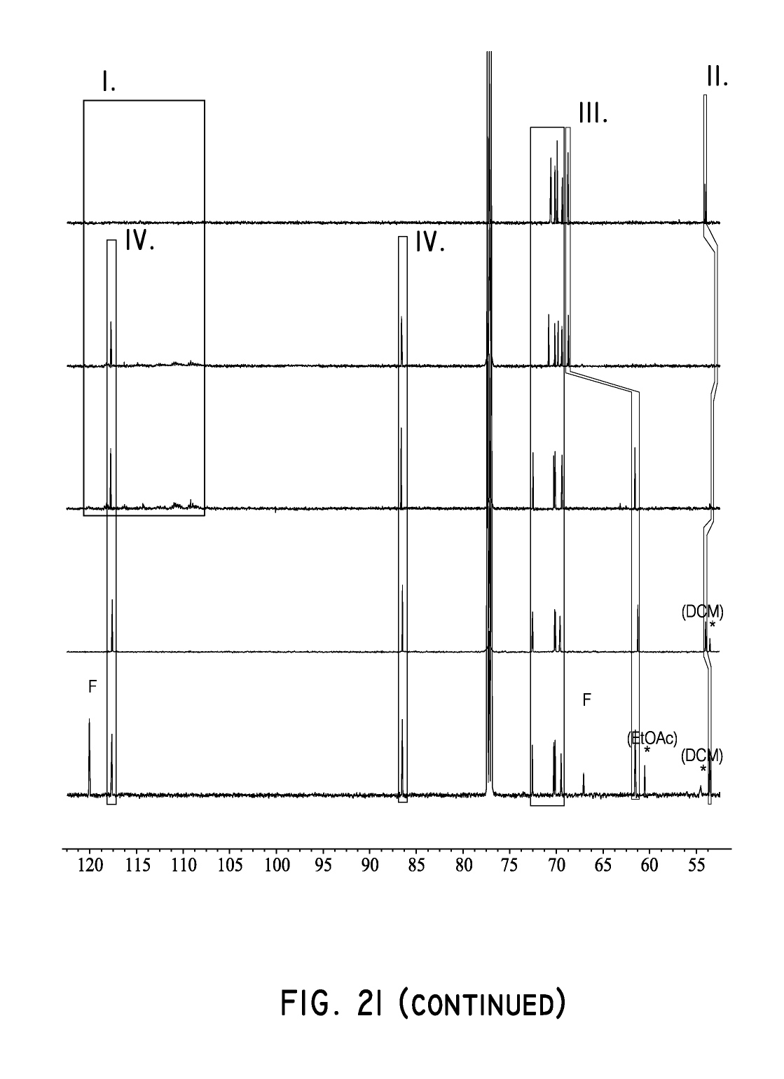

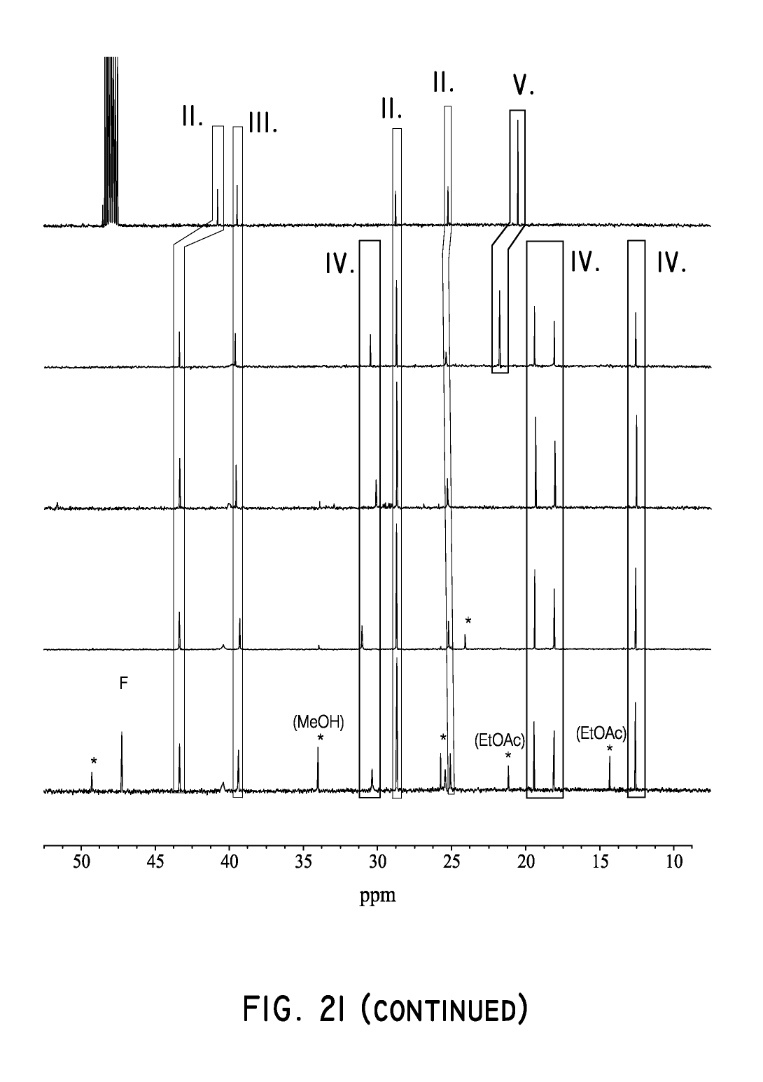

NMR spectra—For comparison, spectra of 14, 15, 16, 1 and 2 are stacked and shown in FIGS. 20 and 21. Carbons (for FIG. 21) and protons (for FIG. 20) attached to the carbon enclosed by the Boxes labeled as I, II, III, IV, and V in the probe (FIG. 19) correspond to the peak enclosed by the same labeled box in FIGS. 20 and 21.

### Alkylation Reactions

The ability of compounds 1 and 2 to react with a number of biologically relevant nucleophiles was investigated. As shown in FIG. 17, 1 was able to alkylate alcohols by means of a Williamson ether synthesis, forming Nimzyme probes 3 and 4. Amines were likewise readily alkylated by 1 to form 5 and 6, provided the free base form was used. Excess nucleophile was used to drive the reactions and to avoid over-alkylation of amines. The alkylated intermediates could be recovered using F-SPE, circumventing cumbersome chromatographic purifications. Subsequent deprotection of the Pbf group affords the desired Nimzyme probes in good yield. Alkylating agent 2 was found to react readily with carboxylate salts and phenolates to directly form Nimzyme probes 7-10. Excess nucleophile was removed using basic aqueous washes.

### Detection of Chloramphenicol Acetyltransferase Activity

To verify that the substrate analogs synthesized according to the described methodology could be used to detect enzymatic activity in a Nimzyme assay, chloramphenicol analog 11 was synthesized as a probe for CAT activity (FIG. 17B-C). CAT catalyzes the transfer of an acetyl group from acetyl-CoA to O³ of chloramphenicol. When 11 is exposed to a control E. coli cell lysate, Nimzyme reveals a clean mass peak corresponding to this substrate (FIG. 17D). When instead 11 is exposed to lysate from E. coli overexpressing CAT, the peak shifts by 42 mass units, as expected from monoacetylation (FIG. 17E). This result shows that the method disclosed herein can be used to a non-carbohydrate-active enzyme.

As shown above, a facile synthesis of a pair of alkylating agents has been performed and the conditions under which these alkylating agents can be used to transform a wide range of structures into Nimzyme probes have been determined.

The method described here allows for the construction of libraries of Nimzyme substrates without the need to repeatedly re-synthesize the invariable portion of the probes. By streamlining access to compatible enzyme substrates, and by showing that Nimzyme can be applied to non-carbohydrate-active enzymes, we hope to have cleared two of the barriers to the widespread adoption of this technology for high-throughput enzyme characterization.

### Example 5

### Tagging Pyruvate to Study Glycomics by Mass Spectrometry

Pyruvate is the key intersection in several metabolic pathways. Therefore, the qualitative and quantitative identification of pyruvates in a crude complex mixture are important for understanding pathway mechanisms, bottle-neck problems etc. Since pyruvate contains a ketone functionality, our aminooxy probe can form oxime with ketone selectively. The resulting oxime can be analyzed by NIMS to provide quantitative data about pyruvate.

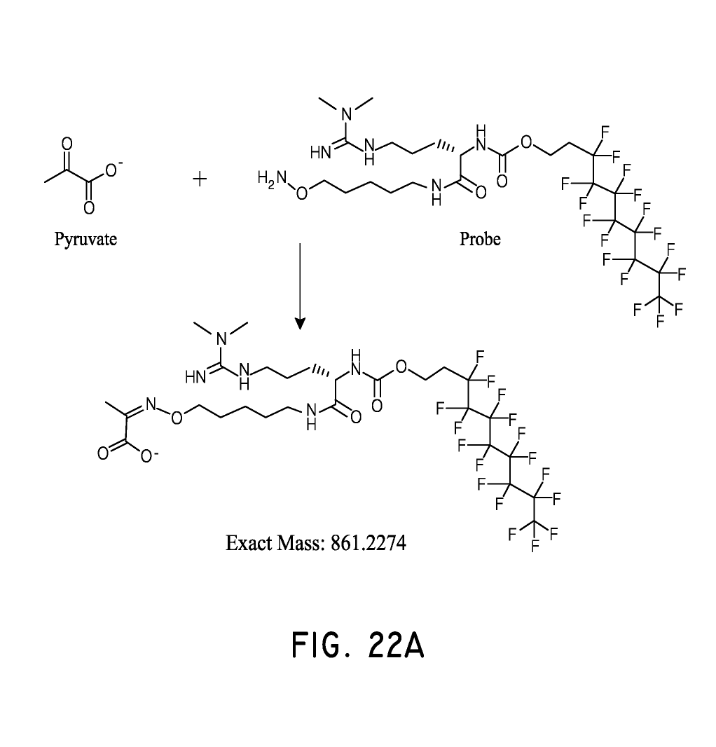

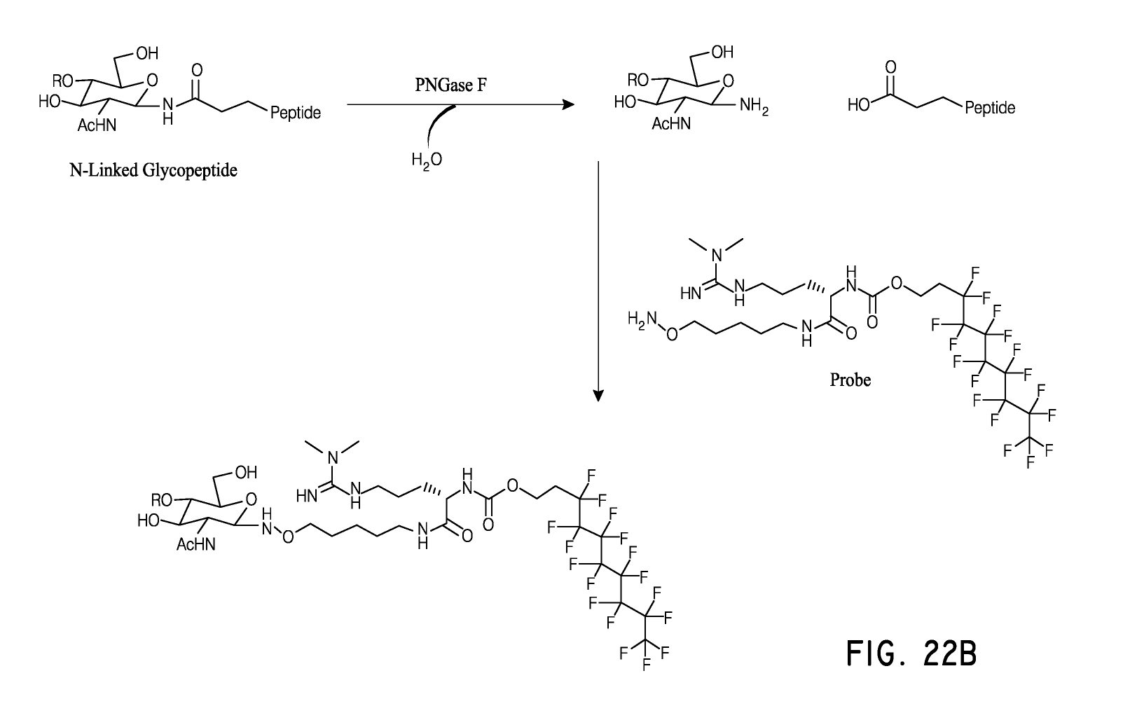

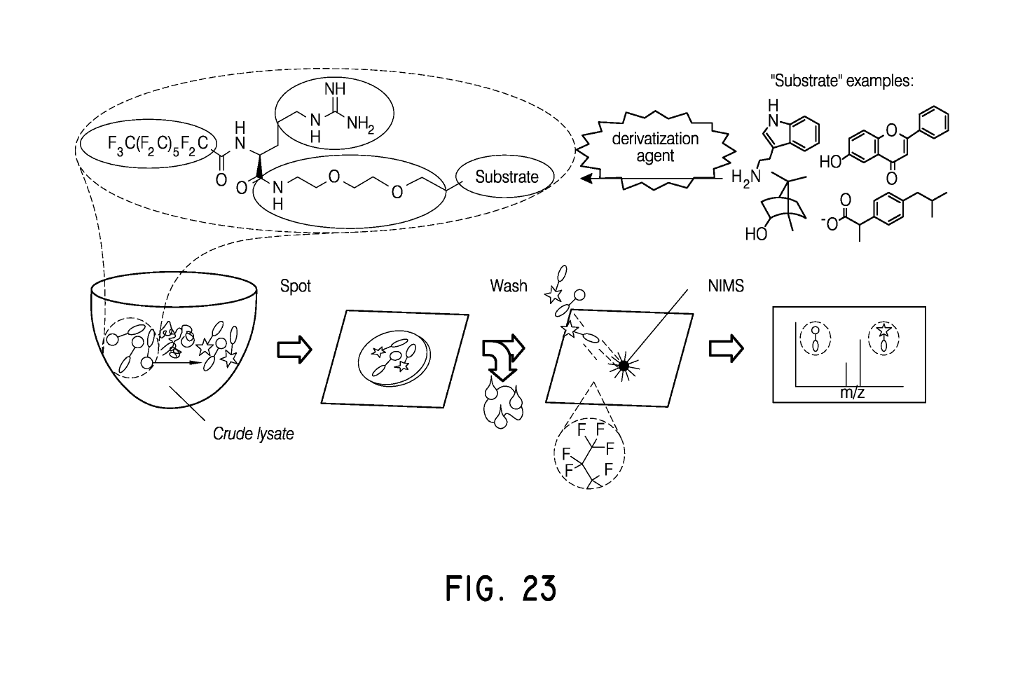

Preliminary results show that product formation could be successfully detected by NIMS, suggesting the mass probe described herein can be used to study glycomics by mass spectrometry. FIG. 22B shows a non-limiting example with an N-linked glycoprotein.

### Example 6

### PECAN (“Probing Enzymes with ‘Click’-Augmented NIMS”) Technology

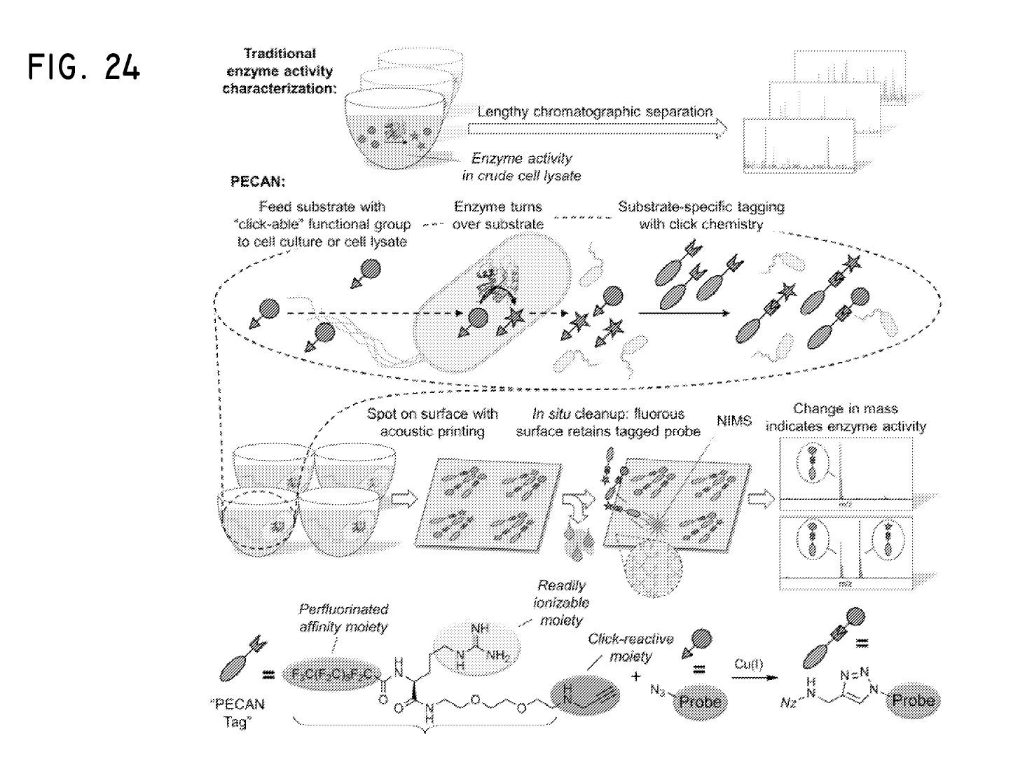

This example describes non-limiting applications of PECAN (“Probing Enzymes with ‘Click’-Augmented NIMS”) technology for detecting enzyme activity. As shown in FIG. 24, in PECAN, a fluorous affinity tag is attached to an enzyme substrate after the enzymatic transformation has taken place using a Copper(I)-catalyzed alkyne-azide cycloaddition. For example, a clickable functional group (˜5 atoms) can be added into the enzyme substrate.

PECAN can be applied to reconstituted enzymes, cell lysate (any type: crude, desalted, clarified) or to whole cells. For example, a substrate (e.g., a hydrophobic substrate) can pass through cell membranes, enabling the ability to detect enzyme activity in whole cells. Probing enzymatic activity in vivo can increase the relevance of screens to downstream whole-cell biocatalysis applications. Compared to testing for enzymatic activity in lysate, PECAN allows for higher experimental throughput by avoiding the lysis procedure, and it is expected to be more cost effective because no exogenous cofactors need to be included.

### Example 7

### Synthesis of PECAN Tags and PECAN Probes

PECAN tags were synthesized from the molecule labeled Nz(Pbf)-OTs or (2) as described in de Rond et al. (Anal. Bioanal. Chem. 2013, 405, 4969-4973).

Nz(Pbf)-OTs was stirred overnight at room temperature in neat propargylamine and purified using a fluorous solid-phase extraction cartridge to form Nz(Pbf)-NPrg, which was stirred in 90:10 trifluoroacetic acid (TFA):methanol overnight at room temperature followed by evaporation of the TFA:methanol to form the Nz-NPrg PECAN tag. Nz(Pbf)-OTs was also stirred in 50 eq sodium azide in DMF overnight at 60° C., quenched with water, and extracted with ethyl acetate to form Nz(Pbf)-N₃, which was stirred in 90:10 TFA:methanol overnight at room temperature followed by evaporation of the TFA:methanol to form the Nz-N₃ PECAN tag.

The PECAN probes (that is, enzyme substrates) used in Examples 8 and 9 were synthesized as follows: leg of (1R,2R)-2-Amino-1-(4-nitrophenyl)-1,3-propanediol (a.k.a. “chloramphenicol base”) and leg of 1-Hexynoic acid were stirred with 1.1 eq PyBOP in dichloromethane (DCM) and purified by silica gel chromatography to afford the chloramphenicol 5-hexynamide (ChlorHexy) PECAN probe.

1 eq of 2-chloroethanol, 1.1 eq of cyclohexenecarboxylic acid, 1.2 eq EDC and 0.25 eq DMAP were stirred in DCM at room temperature to form cyclohexenecarboxylate chloroethyl ester, which was purified by liquid-liquid extraction, and then treated with 5 eq sodium azide in DMF overnight at 80° C., quenched with water, and extracted with ethyl acetate to form the cyclohexenecarboxylate azidoethyl ester (CAEE) PECAN probe.

### Example 8

### Detecting of Chloramphenicol Acetyltransferase Activity Using PECAN

The ability of PECAN to detect chloramphenicol acetyltransferase (CAT) activity was tested. CAT catalyzes the transfer of an acetyl group from acetyl-CoA to O₃ of chloramphenicol:

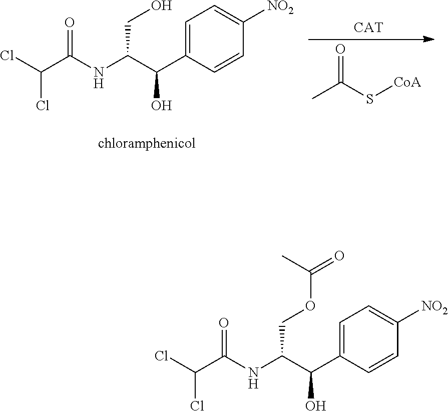

A chloramphenicol analog (labeled ChlorHexy below) was produced as described in Example 7, and used as a PECAN probe to detect CAT enzymatic activity.

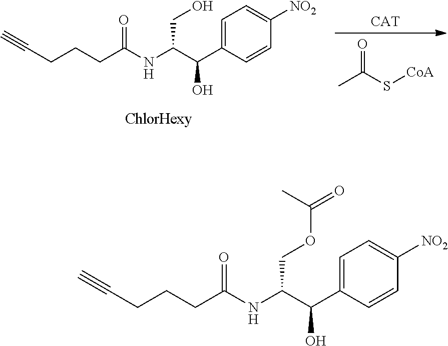

CAT activity was detected in both E. coli cell lysate (with added acetyl-CoA) and in whole E. coli cell culture (without any exogenous cofactors). For the detection of CAT activity E. coli cell lysate, liquid cultures of Escherichia coli DH1 expressing either GFP or CAT were centrifuged, the pellet was resuspended in aqueous 50 mM sodium phosphate (pH 7.5), and sonicated for 30 s. Acetyl-CoA was added to a final concentration of 5 mM and the ChlorHexy PECAN probe to a final concentration of 2 mM. The mixture was mixed and incubated at room temperature for 5 min and subsequently quenched with an equal volume of methanol. 5 uL, of the mixture was then mixed with 5 uL of click reaction solution [50 mM Tris(3-hydroxypropyltriazolylmethyl)amine (THPTA), 50 mM sodium ascorbate, 10 mM copper sulfate and 0.5 mM PECAN tag Nz-N₃ (structure shown below) in 50:50 water:methanol] and left to react overnight. A 0.3-μL droplet of this mixture was then spotted onto a NIMS chip and the excess liquid removed 5 s later by touching it with a Kimwipe (Kimberly-Clark). The spotted area was washed with 2×1 μL deionized water and NIMS spectra were obtained.

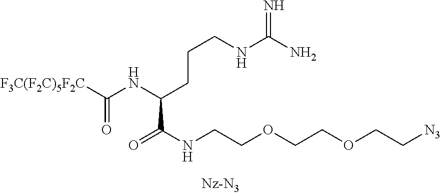

For the detection of CAT activity in E. coli cell culture, to 5 mL, of E. coli culture in LB medium expressing either GFP or CAT was added the ChlorHexy PECAN probe to a final concentration of 2 mM. The bacterial cultures were shaken at 37° C. for 1 h and quenched with an equal volume of methanol. 5 uL of this mixture was then mixed with 5 ul, of click reaction solution (recipe described above) and left to react overnight. NIMS analysis was performed as described above.

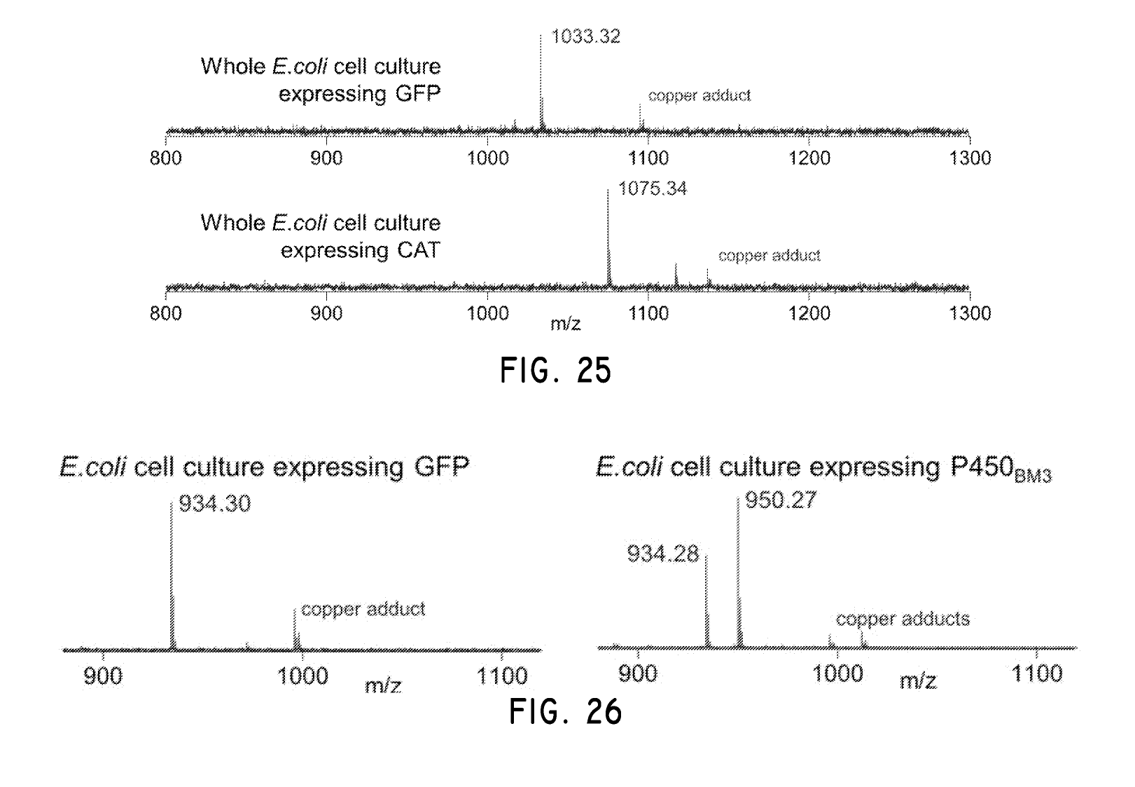

NIMS data obtained for the detection of CAT activity in whole cell culture are shown in FIG. 25. In FIG. 25, the 42-mass unit shift relative to the GFP control was the mass of a single acetyl group attached to the probe.

Since CAT is a common clinical antibiotic resistance mechanism, it is beneficial to develop fast, sensitive and reliable method for detecting CAT activity. As demonstrated in this example, PECAN is able to detect such resistance. The PECAN detection is expected to be faster than current PCR-based detection methods.

### Example 9

### Detecting of Cytochrome P450 Activity Using PECAN

The ability of PECAN to detect cytochrome P450 activity was tested. An analog of a known substrate of cytochrome P450_{BM3} was synthesized and used in a whole-cell PECAN experiment.

A known substrate of cytochrome P450_{BM3} as reported by Agudo et at. (Chembiochem 2012, 13, 1465-1473) is shown below:

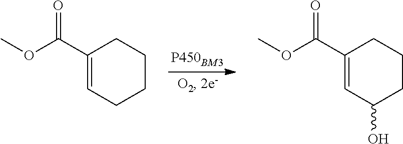

An analog of the above-shown known substrate of cytochrome P450_{BM3} was produced as described in Example 7. The analog was named as CAEE PECAN probe (shown below).

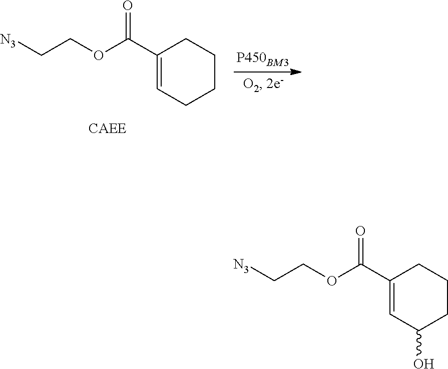 [SMILES: [N-]=[N+]=NCCOC(=O)C1=CCCCC1 ; [N-]=[N+]=NCCOC(=O)C1=CC(O)CCC1]

The CAEE PECAN probe was used to detect cytochrome P450_{BM3} activity in whole E. coli cell culture expressing cytochrome P450_{BM3} using NIMS technology. To 5 mL of E. coli liquid culture in teriffic broth supplemented with 0.5 mM d-aminolevulinic acid, expressing either GFP or cytochrome P450_{BM3}, was added the CAEE PECAN probe to a final concentration of 2 mM and DMSO to a final concentration of 2%. The bacterial cultures were shaken in baffled borosilicate culture tubes at 30° C. for 16 h and quenched with an equal volume of methanol. 5 uL, of this mixture was then mixed with 5 uL, of click reaction solution [50 mM Tris(3-hydroxypropyltriazolylinethyl)amine (THPTA), 50 mM sodium ascorbate, 10 mM copper sulfate and 0.5 mM PECAN tag Nz-NPrg (structure shown below) in 50:50 water:methanol] and left to react overnight. NIMS analysis was performed as described above. The results of the NIMS analysis are shown in FIG. 26. In the NIMS data shown in FIG. 26, the 16-mass unit shift relative to the GFP control was expected to be a single hydroxylation.

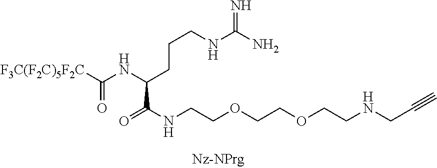

### Example 10

### Detecting of Ketone and Aldehydes Using Oxime-NIMS Technology

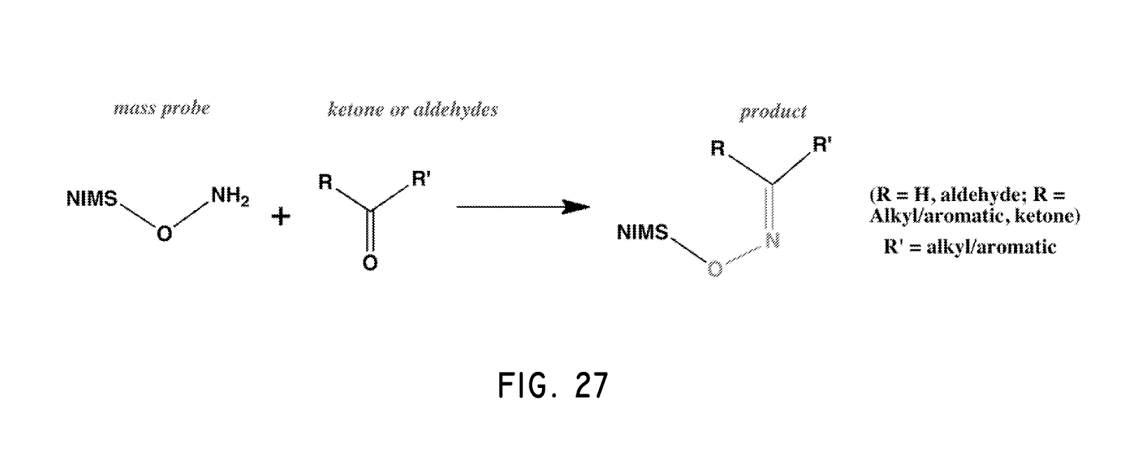

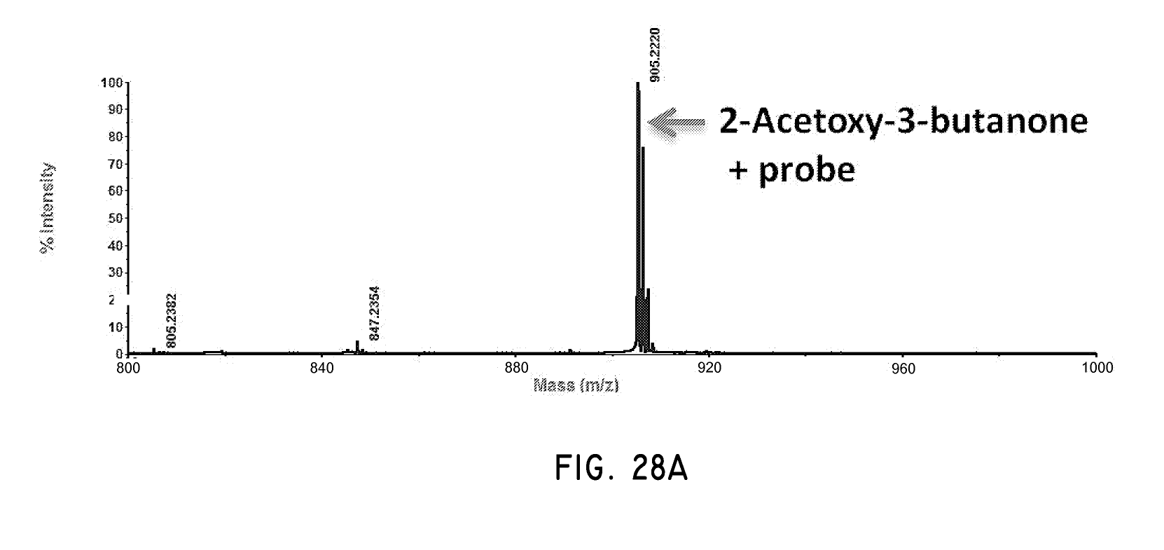

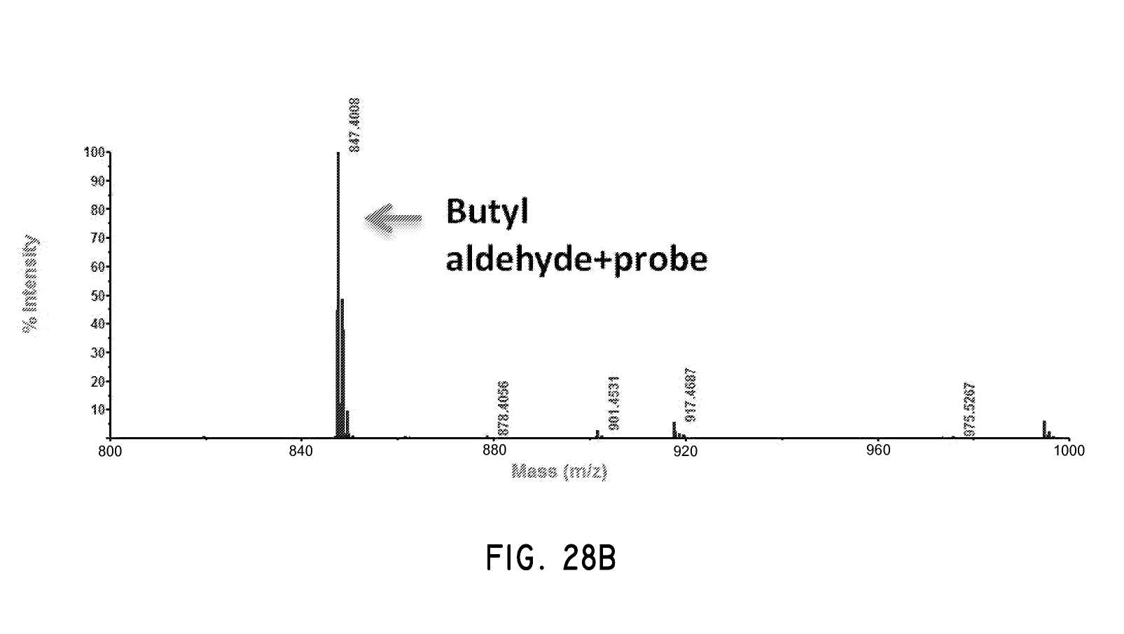

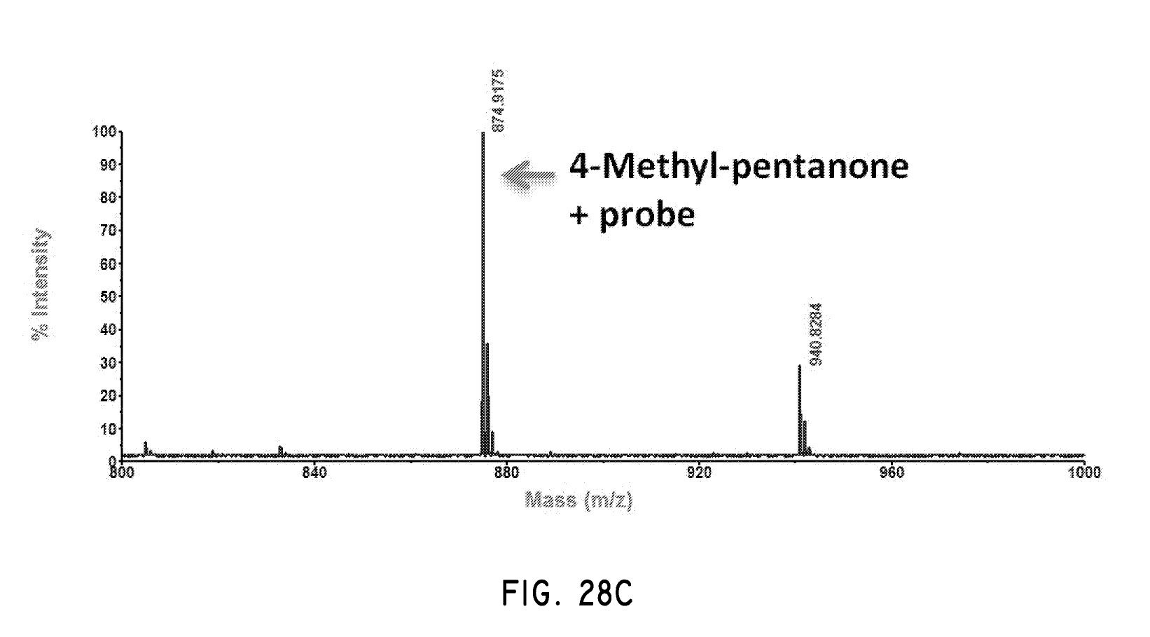

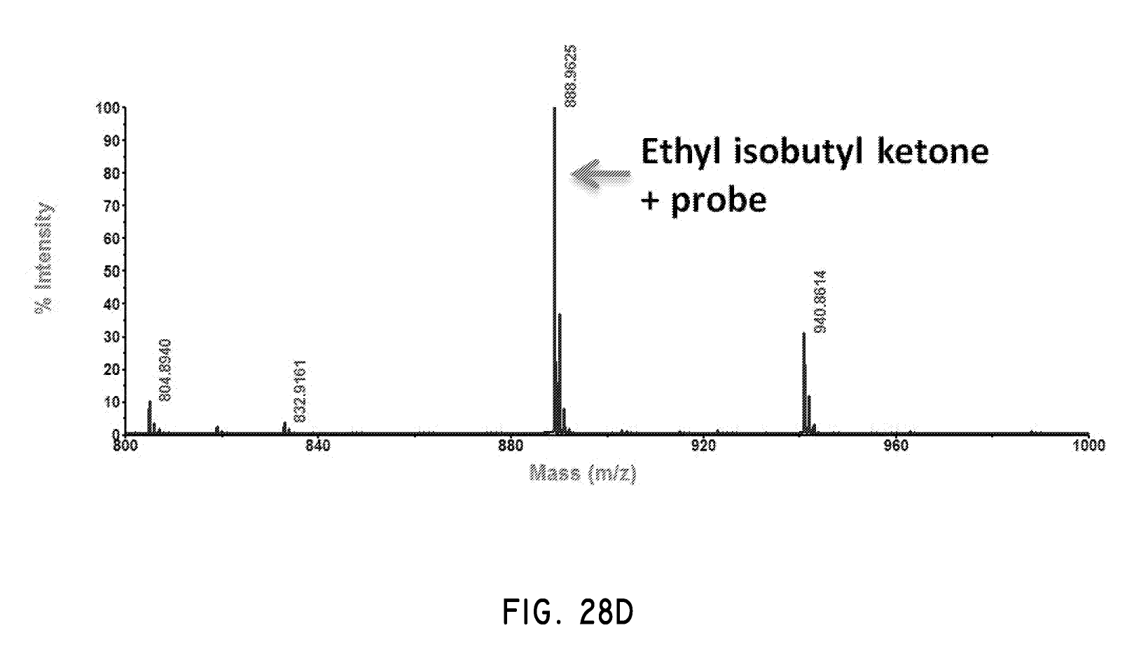

It is expected that the methods for detecting enzyme activity as described herein can be used to detect and analyze any compounds with ketone and/or aldehyde function group (e.g., aldehydes). Without being bound by a particular theory, it is believed that a mass probe having an O-hydroxylamine group can interact with a keton or aldehyde to form a stable oxime derivative via a chemical condensation reaction. A non-limiting illustration of the chemical condensation reaction is shown in FIG. 27. Four compounds: 1) 2-acetoxy-3-butanone, 2) n-butyl aldehyde, 3) Ethyl isobutyl ketone, and 4) 4-methyl-pentanone were used to evaludate oxime tagging by a mass probe having an O-hydroxylaimine group. As shown in FIGS. 28A-D, all four compounds show excellent tagging efficiency and can be quantified by our NIMS-oxime approach.

The above description and examples are provided to illustrate the invention but not to limit its scope. Other variants of the invention will be readily apparent to one of ordinary skill in the art. All publications, databases, and patents cited herein are hereby incorporated by reference for all purposes.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context anchor application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases at least one and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the aft will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention “a system having at least one of A, B, or C” would include but not be limited to systems that have A alone, B alone, C alone, A and B together, A and C together, B and C together, and/or A, B, and C together, etc.). It will he further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for detecting the activity of an enzyme sample, comprising
(a) incubating a substrate sample with an enzyme sample to form a reaction mixture, wherein a substrate sample containing one or more substrates for an enzyme sample, and wherein the reaction mixture contains one or more reaction products;
(b) coupling the one or more reaction products with a mass probe to form tagged reaction products; and
(c) detecting the enzymatic activity of the enzyme sample by analyzing at least one of the one or more reaction products.

2. The method of claim 1, wherein the substrate sample is a biological sample, an environmental sample, or a combination thereof.

3. The method of claim 1, wherein the substrate sample comprises a biomass, a crude lysate, a cell culture, a plant matter, an organic matter, a native glycan, or a combination thereof.

4. The method of claim 1, wherein the substrate sample is a pretreated biomass.

5. The method of claim 1, wherein the substrate sample is a heterogeneous mixture of reaction products from biomass deconstruction.

6. The method of claim 1, wherein at least one of the one or more substrates is a carbohydrate.

7. The method of claim 1, wherein the one or more substrates are not carbohydrates.

8. The method of claim 1, wherein the substrate is an oligosaccharide, a monosaccharide, and a compound with a ketone and/or aldehyde function group.

9. (canceled)

10. The method of claim 1, wherein the enzyme sample comprises a carbohydrate-active enzyme.

11. The method of claim 10, wherein the carbohydrate-active enzyme is a glycoside hydrolase, a polysaccharide lyase, or a glycosyltransferase.

12. The method of claim 10, wherein the carbohydrate-active enzyme is an endoglucanase, an exoglucanase, a pectinase, a cellulase, or a hemicellulase.

13. The method of claim 1, wherein the enzyme sample comprises a non-carbohydrate-active enzyme.

14. (canceled)

15. The method of claim 1, wherein the mass tag is coupled to the reaction product via a triazole moiety.

16. The method of claim 1, wherein the mass tag is coupled to the reaction product by an oxime linage.

17. (canceled)

18. (canceled)

19. The method of claim 1, wherein the mass tag comprises an O-hydroxylamine, an alkyne, or an azide.

20. (canceled)

21. (canceled)

22. (canceled)

23. The method of claim 1, wherein analyzing at least one of the one or more reaction products in step (c) comprises identifying the reaction product, or determining the concentration of the reaction product in the reaction mixture, or both.

24. The method of claim 1, wherein analyzing at least one of the one or more reaction products in step (c) comprises analyzing the reaction product by mass spectrometry.

25. The method of claim 24, wherein the mass spectrometry is nanostructure-initiator mass spectrometry (NIMS).

26. (canceled)

27. (canceled)

28. (canceled)

29. A method for monitoring enzymatic degradation process of a substrate sample, comprising
(a) incubating a substrate sample with an enzyme sample to form a reaction mixture, wherein the reaction mixture contains one or more reaction products;
(b) coupling the one or more reaction products with a mass probe to form tagged reaction products; and
(c) determining the extent of enzymatic degradation of the substrate sample by analyzing at least one of the one or more reaction products.

30. The method of claim 29, wherein the method comprises repeating steps (b) and (c) one or more times to determine the process of enzymatic degradation of the substrate sample.

31. The method of claim 29, further comprising adjusting the composition of the enzyme sample before repeating steps (b) and (c).

32. The method of claim 29, wherein determining the extent of enzymatic degradation of the substrate sample comprises analyzing the at least one of the one or more reaction products.

33. The method of claim 29, wherein the substrate sample is a biomass.

34. (canceled)

35. The method of claim 33, wherein the biomass is pretreated.

36. (canceled)

37. (canceled)

38. (canceled)

39. (canceled)

40. The method of claim 29, wherein analyzing at least one of the one or more reaction products comprises analyzing the reaction product by mass spectrometry.

41. The method of claim 40, wherein the mass spectrometry is nanostructure-initiator mass spectrometry (NIMS).

42. A method for detecting the activities of a plurality of enzymes in a multiplexed assay, comprising
(a) providing a substrate sample containing substrates for a plurality of enzyme;
(b) incubating the substrate sample with the plurality of enzyme to form a reaction mixture, wherein the reaction mixture obtains reaction products;
(c) coupling the reaction products with a mass probe to form tagged reaction products; and
(d) detecting activities of the plurality of enzymes by analyzing the reaction products.

43. (canceled)

44. (canceled)

45. (canceled)

46. (canceled)

47. (canceled)

48. (canceled)
